(19) United States
(12) Patent Application Publication
(10) Pub. No.: US 2022/0339183 A1
(43) Pub. Date: Oct. 27, 2022

(54) TREATMENT OF TAUOPATHIES

(71) Applicant: MACQUARIE UNIVERSITY, North Ryde (AU)

(72) Inventors: Lars ITTNER, North Ryde (AU); Arne ITTNER, North Ryde (AU)

(73) Assignee: MACQUARIE UNIVERSITY, North Ryde (AU)

(21) Appl. No.: 17/754,047

(22) PCT Filed: Sep. 23, 2020
(86) PCT No.: PCT/AU2020/051013

§ 371 Date: Mar. 22, 2022

(30) Foreign Application Priority Data
Sep. 23, 2019 (AU) 2019903530

(51) Int. Cl. A61K 31/713; A61K 39/395; C07K 14/47; A61K 47/64; A61K 9/28; A61P 25/28

(52) U.S. Cl. CPC A61K 31/713; A61K 39/395; C07K 14/4711; A61K 47/6435; A61K 9/282; A61P 25/28

(57) ABSTRACT

The present invention relates to a method of treating a tauopathy in a subject, a method of improving cognitive ability in a subject suffering from cognitive impairment associated with a tauopathy, a method of reducing tau aggregates and neurofibrillary tangles. The method comprises administering an agent which promotes phosphorylation of tau at threonine in the sequence SSPGSPGTPGSRSR of the tau; and/or introduces a phosphomimetic of a phosphorylated tau, wherein the phosphorylated tau is tau that has been phosphorylated at threonine in the sequence SSPGSPGTPGSRSR of the tau.

### FIELD

The present invention relates to a method of treating a tauopathy in a subject, a method of improving cognitive ability in a subject suffering from cognitive impairment associated with a tauopathy, a method of reducing tau aggregates and neurofibrillary tangles, and to compositions and agents for such methods.

### BACKGROUND

Tau protein (also referred to herein at tau), in its normal state, is a highly soluble protein which is abundant in the central nervous system. Under healthy conditions, the tau protein is associated with, and stabilises, microtubules, particularly microtubules of neuronal cells.

Tauopathies are a class of progressive neurodegenerative disorders that are pathologically defined by the presence of abnormal aggregation of hyperphosphorylated tau in neuronal cells of the brain. The abnormal aggregation occurs when tau protein becomes hyperphosphorylated, dissociates from microtubules and forms insoluble aggregates. As the tau aggregates accumulate in the neuronal cells, they form neurofibrillary tangles (NFT), ultimately leading to neuronal cell death and cognitive decline.

Aggregates of hyperphosphorylated tau are involved in the pathogenesis of neurodegenerative diseases such Alzheimer's disease (AD), frontotemporal dementia and other tauopathies. Progression of NFT pathology throughout the brain correlates with disease progression in degenerative disease. However, to date, the mechanism by which these neurofibrillary tangles cause disease is unknown.

Early diagnosis of tauopathies is not usually possible. Current clinical diagnosis relies on clinical history, and symptom progression. As a consequence, a diagnosis of a tauopathy is often only made after onset of symptoms, and usually when the disease is in its advanced stages after significant aggregation of tau has occurred.

To date there are limited options for treating tauopathies after tau aggregation has occurred, such as AD in its advanced stages (i.e., with tau aggregates), or tauopathies which are associated only with aggregation of tau.

What is needed is a method for treating tauopathies in which tau aggregation and NFT formation has already occurred, and/or in which tau aggregation is the only causative factor.

### SUMMARY

The inventors have found that the cognitive decline and other symptoms associated with tauopathies can be reduced by promoting phosphorylation of tau at a threonine in the sequence SSPGSPGTPGSRSR (SEQ ID NO: 7) of the tau, such as threonine corresponding to position 205 of full length wild-type human tau (T205), and/or introducing into neurons of the subject a phosphomimetic of a tau protein that has been phosphorylated at a threonine in the sequence SSPGSPGTPGSRSR (SEQ ID NO: 7) of the tau, such as threonine corresponding to position 205 of full length wild-type human tau (T205) (SEQ ID NO: 1).

A first aspect provides a method of treating or preventing a tauopathy in a subject, comprising administering an agent which:
(a) promotes phosphorylation of tau at threonine in the sequence SSPGSPGTPGSRSR (SEQ ID NO: 7) of the tau; and/or
(b) introduces into neurons of the subject a phosphomimetic of a phosphorylated tau, wherein the phosphorylated tau is tau that has been phosphorylated at threonine in the sequence SSPGSPGTPGSRSR (SEQ ID NO: 7) of the tau.

An alternative first aspect provides an agent for use in treating or preventing a tauopathy in a subject, wherein the agent:

(a) promotes phosphorylation of tau at threonine in the sequence SSPGSPGTPGSRSR of the tau; and/or

(b) introduces into neurons of the subject a phosphomimetic of a phosphorylated tau, wherein the phosphorylated tau is tau that has been phosphorylated at threonine in the sequence SSPGSPGTPGSRSR of the tau; or use of an agent in the manufacture of a medicament for treating or preventing a tauopathy in a subject, wherein the agent:

(a) promotes phosphorylation of tau at threonine in the sequence SSPGSPGTPGSRSR of the tau; and/or

(b) introduces into neurons of the subject a phosphomimetic of a phosphorylated tau, wherein the phosphorylated tau is tau that has been phosphorylated at threonine in the sequence SSPGSPGTPGSRSR of the tau.

A second aspect provides a method of treating or preventing a tauopathy in a subject, comprising administering an agent which:

(a) promotes phosphorylation of tau at threonine corresponding to position 205 of human tau; and/or

(b) introduces into neurons of the subject a phosphomimetic of phosphorylated human tau, wherein the phosphorylated human tau has been phosphorylated at threonine corresponding to position 205 of the tau.

An alternative second aspect provides an agent for use in treating or preventing a tauopathy in a subject, wherein the agent:

(a) promotes phosphorylation of tau at threonine corresponding to position 205 of human tau; and/or

(b) introduces into neurons of the subject a phosphomimetic of phosphorylated human tau, wherein the phosphorylated human tau has been phosphorylated at threonine corresponding to position 205 of the tau; or

use of an agent in the manufacture of a medicament for treating or preventing a tauopathy in a subject, wherein the agent:

(a) promotes phosphorylation of tau at threonine corresponding to position 205 of human tau; and/or

(b) introduces into neurons of the subject a phosphomimetic of phosphorylated human tau, wherein the phosphorylated human tau has been phosphorylated at threonine corresponding to position 205 of the tau.

A third aspect provides a method of treating or preventing a tauopathy in a subject, comprising administering an agent which:

(a) elevates p38γ activity, or the activity of a variant of p38γ, in the neurons of the subject; and/or

(b) introduces in neurons of the subject a nucleotide sequence encoding a phosphomimetic of a phosphorylated tau, wherein the phosphorylated tau is tau that has been phosphorylated at threonine in the sequence SSPGSPGTPGSRSR of the tau, and/or at a threonine corresponding to position 205 of human tau.

An alternative third aspect provides an agent for use in treating or preventing a tauopathy in a subject, wherein the agent:

(a) elevates p38γ activity, or the activity of a variant of p38γ, in the neurons of the subject; and/or

(b) introduces in neurons of the subject a nucleotide sequence encoding a phosphomimetic of a phosphorylated tau, wherein the phosphorylated tau is tau that has been phosphorylated at threonine in the sequence SSPGSPGTPGSRSR of the tau, and/or at a threonine corresponding to position 205 of human tau; or

use of an agent in the manufacture of a medicament for treating or preventing a tauopathy in a subject, wherein the agent:

(a) elevates p38γ activity, or the activity of a variant of p38γ, in the neurons of the subject; and/or

(b) introduces in neurons of the subject a nucleotide sequence encoding a phosphomimetic of a phosphorylated tau, wherein the phosphorylated tau is tau that has been phosphorylated at threonine in the sequence SSPGSPGTPGSRSR of the tau, and/or at a threonine corresponding to position 205 of human tau.

A fourth aspect provides a method of treating or preventing a tauopathy in a subject, comprising administering an agent which elevates p38γ activity, or the activity of a variant of p38γ, in the neurons of the subject.

An alternative fourth aspect provides an agent which elevates p38γ activity, or the activity of a variant of p38γ, in neurons, for use in treating or preventing a tauopathy in a subject; or use of an agent which elevates p38γ activity, or the activity of a variant of p38γ, in neurons, in the manufacture of a medicament for treating or preventing a tauopathy in a subject.

A fifth aspect provides a method of treating or preventing a tauopathy in a subject, comprising administering an agent which alters the nucleotide sequence encoding tau in neurons of the subject such that a phosphomimetic of a phosphorylated tau is expressed, wherein the phosphorylated tau is tau that is phosphorylated at threonine in the sequence SSPGSPGTPGSRSR of the tau, and/or at threonine corresponding to position 205 of human tau.

An alternative fifth aspect provides an agent for use in treating or preventing a tauopathy in a subject, wherein the agent alters the nucleotide sequence encoding tau in neurons of the subject such that a phosphomimetic of a phosphorylated tau is expressed, wherein the phosphorylated tau is tau that is phosphorylated at threonine in the sequence SSPGSPGTPGSRSR of the tau, and/or at threonine corresponding to position 205 of human tau; or use of an agent in the manufacture of a medicament for treating or preventing a tauopathy in a subject, wherein the agent alters the nucleotide sequence encoding tau in neurons of the subject such that a phosphomimetic of a phosphorylated tau is expressed, wherein the phosphorylated tau is tau that is phosphorylated at threonine in the sequence SSPGSPGTPGSRSR of the tau.

A sixth aspect provides a method of treating or preventing a tauopathy, comprising introducing into neurons of the subject:

(a) p38γ, or a variant thereof, or a nucleic acid capable of expressing p38γ, or a variant thereof; or

(b) a nucleic acid capable of altering nucleotide sequence encoding tau such that a phosphomimetic of a phosphorylated tau, wherein the phosphorylated tau is tau that has been phosphorylated at threonine in the sequence SSPGSPGTPGSRSR of the tau, and/or at threonine corresponding to position 205 of human tau, is expressed.

An alternative sixth aspect provides:

(a) p38γ, or a variant thereof, or a nucleic acid capable of expressing p38γ, or a variant thereof; or

(b) a nucleic acid capable of altering nucleotide sequence encoding tau such that a phosphomimetic of a phosphorylated tau, wherein the phosphorylated tau is tau that has been phosphorylated at threonine in the sequence SSPGSPGTPGSRSR of the tau, and/or at threonine corresponding to position 205 of human tau, is expressed,

for use in treating or preventing a tauopathy; or
use of:

(a) p38γ, or a variant thereof, or a nucleic acid capable of expressing p38γ, or a variant thereof; or

(b) a nucleic acid capable of altering nucleotide sequence encoding tau such that a phosphomimetic of a phosphorylated tau, wherein the phosphorylated tau is tau that has been phosphorylated at threonine in the sequence SSPGSPGTPGSRSR of the tau, and/or at threonine corresponding to position 205 of human tau, is expressed,

in the manufacture of a medicament for treating or preventing a tauopathy.

A seventh aspect provides a method of improving cognitive ability in a subject suffering from cognitive impairment associated with a tauopathy, comprising administering to the subject an agent which:

(a) promotes phosphorylation of tau at threonine in the sequence SSPGSPGTPGSRSR of tau; and/or

(b) introduces a phosphomimetic of a phosphorylated tau, wherein the phosphorylated tau is tau that has been phosphorylated at a threonine in the sequence SSPGSPGTPGSRSR of the tau.

An alternative seventh aspect provides an agent for use in improving cognitive ability in a subject suffering from cognitive impairment associated with a tauopathy, wherein the agent:

(a) promotes phosphorylation of tau at threonine in the sequence SSPGSPGTPGSRSR of the tau; and/or

(b) introduces into neurons of the subject a phosphomimetic of a phosphorylated tau, wherein the phosphorylated tau is tau that has been phosphorylated at threonine in the sequence SSPGSPGTPGSRSR of the tau; or

use of an agent in the manufacture of a medicament for improving cognitive ability in a subject suffering from cognitive impairment associated with a tauopathy, wherein the agent:

(a) promotes phosphorylation of tau at threonine in the sequence SSPGSPGTPGSRSR of the tau; and/or

(b) introduces into neurons of the subject a phosphomimetic of a phosphorylated tau, wherein the phosphorylated tau is tau that has been phosphorylated at threonine in the sequence SSPGSPGTPGSRSR of the tau.

An eighth aspect provides a method of improving cognitive ability in a subject suffering from cognitive impairment associated with a tauopathy, comprising administering an agent which:

(a) promotes phosphorylation of tau at a threonine corresponding to position 205 of human tau; and/or

(b) introduces into neurons of the subject a phosphomimetic of phosphorylated human tau, wherein the phosphorylated human tau is human tau that has been phosphorylated at threonine at position 205.

An alternative eighth aspect provides an agent for use in improving cognitive ability in a subject suffering from cognitive impairment associated with a tauopathy, wherein the agent:

(a) promotes phosphorylation of tau at a threonine corresponding to position 205 of human tau; and/or

(b) introduces into neurons of the subject a phosphomimetic of phosphorylated human tau, wherein the phosphorylated human tau has been phosphorylated at threonine at position 205 of the tau; or
use of an agent in the manufacture of a medicament for improving cognitive ability in a subject suffering from cognitive impairment associated with a tauopathy, wherein the agent:

(a) promotes phosphorylation of tau at a threonine corresponding to position 205 of human tau; and/or

(b) introduces into neurons of the subject a phosphomimetic of phosphorylated human tau, wherein the phosphorylated human tau has been phosphorylated at threonine at position 205 of the tau.

A ninth aspect provides a method of improving cognitive ability in a subject suffering from cognitive impairment associated with a tauopathy, comprising administering an agent which:

(a) elevates p38γ activity, or the activity of a variant of p38γ, in the neurons of the subject; and/or

(b) introduces in neurons of the subject a nucleotide sequence encoding a phosphomimetic of a phosphorylated tau, wherein the phosphorylated tau is tau that has been phosphorylated at threonine in the sequence SSPGSPGTPGSRSR of the tau, and/or at a threonine corresponding to position 205 of full-length human tau.

An alternative ninth aspect provides an agent for use in improving cognitive ability in a subject suffering from cognitive impairment associated with a tauopathy, wherein the agent:

(a) elevates p38γ activity, or the activity of a variant of p38γ, in the neurons of the subject; and/or

(b) introduces in neurons of the subject a nucleotide sequence encoding a phosphomimetic of a phosphorylated tau, wherein the phosphorylated tau is tau that has been phosphorylated at threonine in the sequence SSPGSPGTPGSRSR of the tau, and/or at a threonine corresponding to position 205 of full length human tau; or

use of an agent in the manufacture of a medicament for improving cognitive ability in a subject suffering from cognitive impairment associated with a tauopathy, wherein the agent:

(a) elevates p38γ activity, or the activity of a variant of p38γ, in the neurons of the subject; and/or

(b) introduces in neurons of the subject a nucleotide sequence encoding a phosphomimetic of a phosphorylated tau, wherein the phosphorylated tau is tau that has been phosphorylated at threonine in the sequence SSPGSPGTPGSRSR of the tau, and/or at a threonine corresponding to position 205 of full-length human tau.

A tenth aspect provides a method of improving cognitive ability in a subject suffering from cognitive impairment associated with a tauopathy, comprising administering an agent which elevates p38γ activity, or the activity of a variant of p38γ, in the neurons of the subject.

An alternative tenth aspect provides an agent which elevates p38γ activity, or the activity of a variant of p38γ, in neurons, for use in improving cognitive ability in a subject suffering from cognitive impairment associated with a tauopathy; or use of an agent which elevates p38γ activity, or the activity of a variant of p38γ, in neurons, in the manufacture of a medicament for improving cognitive ability in a subject suffering from cognitive impairment associated with a tauopathy.

An eleventh aspect provides a method of improving cognitive ability in a subject suffering from cognitive impairment associated with a tauopathy, comprising administering an agent which alters nucleotide sequence encoding tau in neurons of the subject such that a phosphomimetic of phosphorylated tau is expressed, wherein the phosphorylated tau is tau phosphorylated at threonine in the sequence SSPGSPGTPGSRSR of the tau, and/or at threonine corresponding to position 205 of full-length human tau.

An alternative eleventh aspect provides an agent for use in improving cognitive ability in a subject suffering from cognitive impairment associated with a tauopathy, wherein the agent alters the nucleotide sequence encoding tau in neurons of the subject such that a phosphomimetic of a phosphorylated tau is expressed, wherein the phosphorylated tau is tau that is phosphorylated at threonine in the sequence SSPGSPGTPGSRSR of the tau, and/or at threonine corresponding to position 205 of full length human tau; or use of an agent in the manufacture of a medicament for improving cognitive ability in a subject suffering from cognitive impairment associated with a tauopathy, wherein the agent alters the nucleotide sequence encoding tau in neurons of the subject such that a phosphomimetic of a phosphorylated tau is expressed, wherein the phosphorylated tau is tau that is phosphorylated at threonine in the sequence SSPGSPGTPGSRSR of the tau, and/or at threonine corresponding to position 205 of full-length human tau.

A twelfth aspect provides a method of improving cognitive ability in a subject suffering from cognitive impairment associated with a tauopathy, comprising introducing into neurons of the subject:

(a) p38γ or a variant thereof, or a nucleic acid capable of expressing p38γ, or a variant thereof; and/or

(b) a gene editing system capable of altering nucleotide sequence encoding tau such that a phosphomimetic of a phosphorylated tau is expressed, wherein the phosphorylated tau is tau that has been phosphorylated at threonine in the sequence SSPGSPGTPGSRSR of the tau, and/or at threonine corresponding to position 205 of full length human tau.

An alternative twelfth aspect provides:

(a) p38γ or a variant thereof, or a nucleic acid capable of expressing p38γ, or variant thereof; and/or

(b) a gene editing system capable of altering nucleotide sequence encoding tau such that a phosphomimetic of a phosphorylated tau is expressed, wherein the phosphorylated tau is tau that has been phosphorylated at threonine in the sequence SSPGSPGTPGSRSR of the tau, and/or at threonine corresponding to position 205 of full length human tau,

for use in improving cognitive ability in a subject suffering from cognitive impairment associated with a tauopathy; or

use of:

(a) p38γ or a variant thereof, or a nucleic acid capable of expressing p38γ, or variant thereof; and/or

(b) a gene editing system capable of altering nucleotide sequence encoding tau such that a phosphomimetic of a phosphorylated tau is expressed, wherein the phosphorylated tau is tau that has been phosphorylated at threonine in the sequence SSPGSPGTPGSRSR of the tau, and/or at threonine corresponding to position 205 of full length human tau,

in the manufacture of a medicament for improving cognitive ability in a subject suffering from cognitive impairment associated with a tauopathy.

A thirteenth aspect provides a method of reducing or preventing tau aggregation in neurons, comprising introducing into the neurons an agent which:

(a) promote phosphorylation of tau at threonine in the sequence SSPGSPGTPGSRSR of the tau; and/or

(b) introduce a phosphomimetic of a phosphorylated tau, wherein the phosphorylated tau is tau that has been phosphorylated at threonine in the sequence SSPGSPGTPGSRSR of the tau.

An alternative thirteenth aspect provides an agent for use in reducing or preventing tau aggregation in neurons, wherein the agent:

(a) promotes phosphorylation of tau at threonine in the sequence SSPGSPGTPGSRSR of the tau; and/or

(b) introduces into neurons of the subject a phosphomimetic of a phosphorylated tau, wherein the phosphorylated tau is tau that has been phosphorylated at threonine in the sequence SSPGSPGTPGSRSR of the tau; or

use of an agent in the manufacture of a medicament for reducing or preventing tau aggregation in neurons of a subject, wherein the agent:

(a) promotes phosphorylation of tau at threonine in the sequence SSPGSPGTPGSRSR of the tau; and/or

(b) introduces into neurons of the subject a phosphomimetic of a phosphorylated tau, wherein the phosphorylated tau is tau that has been phosphorylated at threonine in the sequence SSPGSPGTPGSRSR of the tau.

A fourteenth aspect provides a method of reducing or preventing tau aggregation in neurons, comprising introducing into the neurons an agent which:

(a) promotes phosphorylation of tau at a threonine corresponding to position 205 of human tau; and/or

(a) introduces into neurons of the subject a phosphomimetic of phosphorylated human tau, wherein the phosphorylated human tau has been phosphorylated at threonine corresponding to position 205 of the tau.

An alternative fourteenth aspect provides an agent for use in reducing or preventing tau aggregation in neurons, wherein the agent:

(a) promotes phosphorylation of tau at a threonine corresponding to position 205 of human tau; and/or

(b) introduces into neurons of the subject a phosphomimetic of phosphorylated human tau, wherein the phosphorylated human tau has been phosphorylated at threonine corresponding to position 205 of the tau; or

use of an agent in the manufacture of a medicament for reducing or preventing tau aggregation in neurons of a subject, wherein the agent:

(a) promotes phosphorylation of tau at a threonine corresponding to position 205 of human tau; and/or

(b) introduces into neurons of the subject a phosphomimetic of phosphorylated human tau, wherein the phosphorylated human tau has been phosphorylated at threonine corresponding to position 205 of the tau.
A fifteenth aspect provides a method of reducing or preventing tau aggregation in neurons, comprising introducing into the neurons an agent which:

(a) elevates p38γ activity, or the activity of a variant of p38γ, in the neurons of the subject; and/or

(b) introduces in neurons of the subject a nucleotide sequence encoding a phosphomimetic of a phosphorylated tau, wherein the phosphorylated tau is tau that has been phosphorylated at threonine in the sequence SSPGSPGTPGSRSR of the tau, and/or at a threonine corresponding to position 205 of full-length human tau.

An alternative fifteenth aspect provides an agent for use in reducing or preventing tau aggregation in neurons, wherein the agent:

(a) elevates p38γ activity, or the activity of a variant of p38γ, in the neurons of the subject; and/or

(b) introduces in neurons of the subject a nucleotide sequence encoding a phosphomimetic of a phosphorylated tau, wherein the phosphorylated tau is tau that has been phosphorylated at threonine in the sequence SSPGSPGTPGSRSR of the tau, and/or at a threonine corresponding to position 205 of full-length human tau; or
use of an agent in the manufacture of a medicament for reducing or preventing tau aggregation in neurons of a subject, wherein the agent:

(a) elevates p38γ activity, or the activity of a variant of p38γ, in the neurons of the subject; and/or

(b) introduces in neurons of the subject a nucleotide sequence encoding a phosphomimetic of a phosphorylated tau, wherein the phosphorylated tau is tau that has been phosphorylated at threonine in the sequence SSPGSPGTPGSRSR of the tau, and/or at a threonine corresponding to position 205 of full-length human tau.

A sixteenth aspect provides a method of reducing or preventing tau aggregation in neurons of a subject, comprising introducing into neurons of the subject:

(a) p38γ, or a variant thereof, or a nucleic acid capable of expressing p38γ, or a variant thereof; and/or

(b) a nucleic acid capable of altering nucleotide sequence encoding tau such that a phosphomimetic of a phosphorylated tau, wherein the phosphorylated tau is tau that has been phosphorylated at threonine in the sequence SSPGSPGTPGSRSR of the tau, and/or at threonine corresponding to position 205 of human tau, is expressed.

An alternative sixteenth aspect provides:

(a) p38γ, or a variant thereof, or a nucleic acid capable of expressing p38γ, or a variant thereof; and/or

(b) a nucleic acid capable of altering nucleotide sequence encoding tau such that a phosphomimetic of a phosphorylated tau, wherein the phosphorylated tau is tau that has been phosphorylated at threonine in the sequence SSPGSPGTPGSRSR of the tau, and/or at threonine corresponding to position 205 of human tau, is expressed,

for use in reducing or preventing tau aggregation in neurons of a subject; or
use of:

(a) p38γ, or a variant thereof, or a nucleic acid capable of expressing p38γ, or a variant thereof; and/or

(b) a nucleic acid capable of altering nucleotide sequence encoding tau such that a phosphomimetic of a phosphorylated tau, wherein the phosphorylated tau is tau that has been phosphorylated at threonine in the sequence SSPGSPGTPGSRSR of the tau, and/or at threonine corresponding to position 205 of human tau, is expressed,

in the manufacture of a medicament for reducing or preventing tau aggregation in neurons of a subject.

A seventeenth aspect provides a method of reducing or preventing neurofibrillary tangles in neurons of a subject, comprising administering an effective amount of an agent which:

(a) promote phosphorylation of tau at threonine in the sequence SSPGSPGTPGSRSR of the tau; and/or

(b) introduce a phosphomimetic of a phosphorylated tau, wherein the phosphorylated tau is tau that has been phosphorylated at a threonine in the sequence SSPGSPGTPGSRSR of the tau.

An alternative seventeenth aspect provides an agent for use in reducing or preventing neurofibrillary tangles in neurons of a subject, wherein the agent:

(a) promotes phosphorylation of tau at threonine in the sequence SSPGSPGTPGSRSR of the tau; and/or

(b) introduces into neurons of the subject a phosphomimetic of a phosphorylated tau, wherein the phosphorylated tau is tau that has been phosphorylated at threonine in the sequence SSPGSPGTPGSRSR of the tau; or

use of an agent in the manufacture of a medicament for reducing or preventing neurofibrillary tangles in neurons of a subject, wherein the agent:

(a) promotes phosphorylation of tau at threonine in the sequence SSPGSPGTPGSRSR of the tau; and/or

(b) introduces into neurons of the subject a phosphomimetic of a phosphorylated tau, wherein the phosphorylated tau is tau that has been phosphorylated at threonine in the sequence SSPGSPGTPGSRSR of the tau.

An eighteenth aspect provides a method of reducing or preventing neurofibrillary tangles in neurons of a subject, comprising administering an effective amount of an agent which:

(a) promotes phosphorylation of tau at a threonine corresponding to position 205 of human tau; and/or

(c) introduces into neurons of the subject a phosphomimetic of phosphorylated human tau, wherein the phosphorylated human tau has been phosphorylated at threonine corresponding to position 205 of the tau.

An alternative eighteenth aspect provides an agent for use in reducing or preventing neurofibrillary tangles in neurons of a subject, wherein the agent:

(a) promotes phosphorylation of tau at a threonine corresponding to position 205 of human tau; and/or

(b) introduces into neurons of the subject a phosphomimetic of phosphorylated human tau, wherein the phosphorylated human tau has been phosphorylated at threonine corresponding to position 205 of the tau; or

use of an agent in the manufacture of a medicament for reducing or preventing neurofibrillary tangles in neurons of a subject, wherein the agent:

(a) promotes phosphorylation of tau at a threonine corresponding to position 205 of human tau; and/or

(b) introduces into neurons of the subject a phosphomimetic of phosphorylated human tau, wherein the phosphorylated human tau has been phosphorylated at threonine corresponding to position 205 of the tau.

A nineteenth aspect provides a method of reducing or preventing neurofibrillary tangles in neurons of a subject, comprising introducing into the neurons an agent which:

(a) elevates p38γ activity, or the activity of a variant of p38γ, in the neurons of the subject; and/or

(b) introduces in neurons of the subject a nucleotide sequence encoding a phosphomimetic of a phosphorylated tau, wherein the phosphorylated tau is tau that has been phosphorylated at threonine in the sequence SSPGSPGTPGSRSR of the tau, and/or at a threonine corresponding to position 205 of full-length human tau.

An alternative nineteenth aspect provides an agent for use in reducing or preventing neurofibrillary tangles in neurons of a subject, wherein the agent:

(a) elevates p38γ activity, or the activity of a variant of p38γ, in the neurons of the subject; and/or

(b) introduces in neurons of the subject a nucleotide sequence encoding a phosphomimetic of a phosphorylated tau, wherein the phosphorylated tau is tau that has been phosphorylated at threonine in the sequence SSPGSPGTPGSRSR of the tau, and/or at a threonine corresponding to position 205 of full-length human tau; or

use of an agent in the manufacture of a medicament for reducing or preventing neurofibrillary tangles in neurons of a subject, wherein the agent:

(a) elevates p38γ activity, or the activity of a variant of p38γ, in the neurons of the subject; and/or

(b) introduces in neurons of the subject a nucleotide sequence encoding a phosphomimetic of a phosphorylated tau, wherein the phosphorylated tau is tau that has been phosphorylated at threonine in the sequence SSPGSPGTPGSRSR of the tau, and/or at a threonine corresponding to position 205 of full-length human tau.

A twentieth aspect provides a method of reducing or preventing neurofibrillary tangles in neurons of a subject, comprising introducing into neurons of the subject:

(a) p38γ, or a variant thereof, or a nucleic acid capable of expressing p38γ, or a variant thereof; and/or

(b) a nucleic acid capable of altering nucleotide sequence encoding tau such that a phosphomimetic of a phosphorylated tau, wherein the phosphorylated tau is tau that has been phosphorylated at threonine in the sequence SSPGSPGTPGSRSR of the tau, and/or at threonine corresponding to position 205 of human tau, is expressed.

An alternative twentieth aspect provides:

(a) p38γ, or a variant thereof, or a nucleic acid capable of expressing p38γ, or a variant thereof; and/or

(b) a nucleic acid capable of altering nucleotide sequence encoding tau such that a phosphomimetic of a phosphorylated tau, wherein the phosphorylated tau is tau that has been phosphorylated at threonine in the sequence SSPGSPGTPGSRSR of the tau, and/or at threonine corresponding to position 205 of human tau, is expressed,

for use in reducing or preventing neurofibrillary tangles in neurons of a subject; or
use of:

(a) p38γ, or a variant thereof, or a nucleic acid capable of expressing p38γ, or a variant thereof; and/or

(b) a nucleic acid capable of altering nucleotide sequence encoding tau such that a phosphomimetic of a phosphorylated tau, wherein the phosphorylated tau is tau that has been phosphorylated at threonine in the sequence SSPGSPGTPGSRSR of the tau, and/or at threonine corresponding to position 205 of human tau, is expressed, in the manufacture of a medicament for reducing or preventing neurofibrillary tangles in neurons of a subject.

A twenty first aspect provides a method of reducing phosphorylation of serine at position 422 of human tau in a subject suffering from a tauopathy, the method comprising administering an effective amount of an agent which:

(a) promotes phosphorylation of tau at threonine in the sequence SSPGSPGTPGSRSR of the tau; and/or

(b) introduce a phosphomimetic of a phosphorylated tau, wherein the phosphorylated tau is tau that has been phosphorylated at threonine in the sequence SSPGSPGTPGSRSR of the tau.

An alternative twenty first aspect provides an agent for reducing phosphorylation of serine at position 422 of human tau in a subject suffering from a tauopathy, wherein the agent:

(a) promotes phosphorylation of tau at threonine in the sequence SSPGSPGTPGSRSR of the tau; and/or

(b) introduces into neurons of the subject a phosphomimetic of a phosphorylated tau, wherein the phosphorylated tau is tau that has been phosphorylated at threonine in the sequence SSPGSPGTPGSRSR of the tau; or

use of an agent in the manufacture of a medicament for reducing phosphorylation of serine at position 422 of human tau in a subject suffering from a tauopathy, wherein the agent:

(a) promotes phosphorylation of tau at threonine in the sequence SSPGSPGTPGSRSR of the tau; and/or

(b) introduces into neurons of the subject a phosphomimetic of a phosphorylated tau, wherein the phosphorylated tau is tau that has been phosphorylated at threonine in the sequence SSPGSPGTPGSRSR of the tau.

A twenty second aspect provides a method of treating or preventing a tauopathy associated with phosphorylation of serine at position 422 of human tau in a subject, the method comprising administering an effective amount of an agent which:

(a) promotes phosphorylation of tau at threonine in the sequence SSPGSPGTPGSRSR of the tau; and/or

(b) introduce a phosphomimetic of a phosphorylated tau, wherein the phosphorylated tau is tau that has been phosphorylated at a threonine in the sequence SSPGSPGTPGSRSR of the tau.

An alternative twenty second aspect provides an agent for treating or preventing a tauopathy associated with phosphorylation of serine at position 422 of human tau in a subject, wherein the agent:

(a) promotes phosphorylation of tau at threonine in the sequence SSPGSPGTPGSRSR of the tau; and/or

(b) introduces into neurons of the subject a phosphomimetic of a phosphorylated tau, wherein the phosphorylated tau is tau that has been phosphorylated at threonine in the sequence SSPGSPGTPGSRSR of the tau; or

use of an agent in the manufacture of a medicament for treating or preventing a tauopathy associated with phosphorylation of serine at position 422 of human tau in a subject, wherein the agent:

(a) promotes phosphorylation of tau at threonine in the sequence SSPGSPGTPGSRSR of the tau; and/or

(b) introduces into neurons of the subject a phosphomimetic of a phosphorylated tau, wherein the phosphorylated tau is tau that has been phosphorylated at threonine in the sequence SSPGSPGTPGSRSR of the tau.

A twenty third aspect provides an agent comprising a tau gene (Mapt gene) editing system, or part thereof, or a nucleic acid encoding a tau gene (Mapt gene) editing system, or part thereof, the gene editing system or part thereof comprising one or more nucleic acids which introduce a mutation into nucleotide sequence encoding wild-type tau to cause expression of a phosphomimetic of phosphorylated tau, wherein the phosphorylated tau is tau that has been phosphorylated at threonine in the sequence SSPGSPGTPGSRSR of the tau and/or at threonine corresponding to position 205 of full length human tau.

### BRIEF DESCRIPTION OF THE DRAWINGS

FIG. 1. Delivery of active p38γ MAP kinase improves cognitive performance of Alzheimer's mice at advanced stage. FIG. 1(a) is an experimental schematic diagram showing the timeline of AAV9/PHP.B syn-p38γ^{CA} and control syn-eGFP delivery (intravenous (i.v.), 100 μl) to achieve neuronal expression in 13-month-old APP23 (and non-transgenic control) mice. Two different AAV titres (10¹¹ or 10¹³ virion particles/ml) were used in different experiments. Cognitive performance and histological and biochemical data were assessed 2 months post-delivery of AAV. (b) is an image showing immunofluorescence analysis of cortex and hippocampus of APP23 mice confirming AAV-mediated expression of active p38γ^{CA} (HA) in neurons and transgenic expression of APP (6E10). Scale bar, 50 μm. FIG. 1(c-g) show memory assessment of 13-month-old APP23 mice 2 months post-delivery of AAV9/PHP.B syn-p38γ^{CA} and control syn-eGFP delivery (i.v., 100 μl; 10¹¹ viral genomes per ml) in the Morris water maze (n=10-11). (c) is a diagram showing acquisition phase: representative swim path traces on day 6. (d) is a graph showing escape latencies during acquisition phase (day 1-6) for APP23 mice with indicated AAV treatment. (e) is a graph showing linear regression of acquisition curves in (C). (f) is a graph showing learning curve comparisons. (g) is a graph showing probe trial (day 7) occupancy of water maze quadrants. Q1, target quadrant. Dashed line, threshold of random occupancy. Figures (h-l) Morris water maze latency in 13-month-old APP23 mice 2 months post-delivery of indicated AAVs (i.v., 100 μl; 10¹³ viral genomes per ml). (n=9 for APP23 AAV^{eGFP} and APP23 AAV^{p38γCA}; n=5-6 for controls AAV^{eGFP} and AAV^{p38γCA}). (h) is a diagram showing acquisition phase: representative swim path traces on day 6. (i) is a graph showing escape latencies during acquisition phase (j) is a graph showing linear regression of acquisition curves in (i). (k) is a graph showing learning curve comparisons. (l) is a graph showing probe trial (day 7) occupancy of water maze quadrants. Q1, target quadrant. Data are expressed as mean±S.E.M. **p<0.01 *p<0.05 ns, not significant. (2-way ANOVA for d, i; ANOVA for f, g, k, l).

FIG. 2. Tau toxicity is modulated by endogenous p38γ and Threonine-205 (T205) phosphorylation levels. (a) is a schematic diagram indicating human wildtype tau transgenic mice (Alz17) were crossed with p38γ knockout (p38γ^{-/-}) mice to achieve reduced levels of tau phosphorylation at T205 tau and its cognate kinase p38γ. (b) is an image showing immunofluorescence staining of tau and p38γ cortical sections from Alz17.p38γ^{+/+} and Alz17.p38γ^{-/-} mice. Scale bar, 10 μm. FIGS. 2(c-e) show memory/cognitive performance in the Morris water maze in 10-month-old Alz17.p38γ^{+/+} Alz17.p38γ^{-/-}, p38γ^{+/+} and p38γ^{-/-} control mice. (n=10). (c) is diagrams showing acquisition phase: representative swim path traces on day 6. (d) is a graph showing escape latency on days 1-6 in mice with indicated genotypes. (e) is a graph showing learning curve comparisons by linear regression slopes. (f) is a graph showing Morris water maze quadrant occupation during probe trial on day 7 in mice with indicated genotypes. Q1, target quadrant. Dashed line, random occupancy threshold. Data are expressed as mean±S.E.M. **p<0.01 *p<0.05 ns, not significant. (2-way ANOVA for d; ANOVA for e, f)

FIG. 3. Phosphorylation of endogenous tau at T205 modulates excitotoxic signalling. (a) is a schematic diagram showing generation of tauT205^{A} and tauT205^{E} mice by genome editing. (b) is DNA sequencing chromatograms showing successful codon editing of the tau (Mapt) gene encoding T205 residue in tauT205^{A/A} and tauT205^{E/E} mice as compared with wildtype (tauT205^{T/T}). (c) is an image showing immunoprecipitation of endogenous tau from cortical lysates from tauT205^{T/T} and tauT205^{A/A} mice using anti-tau (tau5) antibody detected with p-T205-specific tau antibody. (d-e) shows that tauT205^{E/E} mice are protected from excitotoxic seizures. (d) is a graph showing seizure latency. (e) is a graph showing mean seizure severity, in tauT205^{T/T} and tauT205^{E/E} mice induced by pentylenetetrazole (PTZ; 50 mg/kg, i.p.). (n=20-22) FIGS. 3(f-g) show that tauT205^{A/A} mice are more susceptible to excitotoxic seizures. (f) is a graph showing seizure latency. (g) is a graph showing mean seizure severity, in tauT205^{T/T} and tauT205^{A/A} mice induced by pentylenetetrazole (PTZ; 30 mg/kg, i.p.). (n=9-12). Data are expressed as mean±S.E.M. ***p<0.001 **p<0.01 *p<0.05 ns, not significant. (2-way ANOVA for e; ANOVA for f)

FIG. 4. Phosphorylation of endogenous tau at T205 modulates cognitive deficits in APP23 mice. (a) is a schematic diagram showing APP23 mice were crossed with either tauT205^{A/A} or tauT205^{E/E} mice to obtain APP23.tauT205^{A/A} and APP23.tauT205^{E/E} mice, respectively. (b) is an image showing immunofluorescence staining of phospho-T205 tau and human APP (6E10) in cortex of APP23.tauT205^{T/T} and APP23.tauT205^{A/A} mice. DAPI, nuclear marker. Scale bare, 50 pm. (c) is a graph showing survival curves of APP23.tauT205^{T/T} (n=72), APP23.tauT205^{A/A} (n=55), and APP23.tauT205^{E/E} (n=38). (e-h) shows the results of Morris water maze in APP23.tauT205^{T/T} and APP23.tauT205^{A/A} as well as tauT205^{T/T} and tauT205^{A/A} mice. (n=6-10). (e) is a diagram showing acquisition phase: representative swim path traces on day 4. (f) is a graph showing escape latencies during learning on days 1-6. (g) is a graph showing escape latency curve comparison. (h) is a graph showing Morris water maze quadrant occupancy during probe trial (day 7). Q1, target quadrant. Dashed line, random occupancy threshold. FIGS. 4(i-l) shows the results of Morris water maze in APP23.tauT205^{T/T} and APP23.tauT205^{E/E} as well as tauT205^{T/T} and tauT205^{E/E} mice. (n=6-10). (i) is a diagram showing acquisition phase: traces on day 6. (j) is a graph showing escape latencies during learning on days 1-6 in APP23.tauT205^{T/T} and APP23.tauT205^{E/E} as well as tauT205^{T/T} and tauT205^{E/E} mice. (k) is a graph showing escape latency curve comparison. (l) is a graph showing Morris water maze quadrant occupancy during probe trial results on day 7 in in APP23.tauT205^{T/T} and APP23.tauT205^{E/E} as well as tauT205^{T/T} and tauT205^{E/E} mice. Q1, target quadrant. Dashed line, random occupancy threshold. Data are expressed as mean±S.E.M. *** p<0.001 **p<0.01 *p<0.05 ns, not significant. (Mantel-Cox test for c, d; 2-way ANOVA for f, j; ANOVA for g, h, k, l)

FIG. 5. T205 in tau is required to mediate protective effect of p38γ activity in a mouse model of Alzheimer's disease. (a) is a schematic diagram showing intercrosses to obtain APP23.tau^{-/-}.p38γ^{CA} mice to test requirement of T205 for protective effects of p38γ in APP23 mice. APP23 mice were crossed with p38γ^{CA} mice and tau^{-/-} mice. (b) is an experimental schematic diagram:APP23.tau^{-/-}.p38γ^{C/A} mice were injected intracranially at postnatal day 0 (P0) with AAV to achieve neuronal expression of either tauWT or tauT205A or eGFP control. AAV-mediated tau expression and learning/memory performance in the Morris water maze were addressed at 6 months. (c) is an image showing immunofluorescence staining for p38γ^{CA} (HA) and human tau on cortical sections from APP23.tau^{-/-}.p38γ^{CA} mice injected with indicated AAV vectors. DAPI, nuclear marker. Scale bare, 50 μm. (n=5) FIGS. 5(d-f) show the results of memory/cognitive performance assessed using Morris water maze in APP23.tau^{-/-}.p38γ^{CA} mice with neuronal expression of tauWT or tauT205A. (n numbers as indicated in label legend). (d) is a graph showing acquisition phase: representative swim path traces on day 4. (e) is a graph showing acquisition phase: escape latencies. (f) is a graph showing Morris water maze: probe trial results on day 8. Q1, target quadrant. Dashed line, random occupancy threshold. Data are expressed as mean±S.E.M. **p<0.01 *p<0.05 ns, not significant. (2-way ANOVA for d; ANOVA for e, f).

FIG. 6. Control parameters for cognitive tests in APP23 treated with AAV p38γ CA. (a, b) are images of immunofluorescence analysis of cortex (a) and hippocampus (b) of APP23 mice confirming AAV-mediated expression of active p38γ in neurons. Scale bar, 50 μm.

FIG. 7. No enhanced tau pathology in tau transgenic mice lacking p38γ. (a) is an image showing immunofluorescence staining of tau and p38γ cortical sections from Alz17.p38^{+/+} and Alz17.p38^{-/-} mice. Scale bar, 10 μm. (b) is a graph showing Morris water maze visual cue test on day 8 in 10-month-old Alz17.p38γ^{+/+}, Alz17.p38γ^{-/-}, p38γ^{+/+} and p38γ^{-/-} control mice. (n=10). (c) is a graph showing Morris water maze quadrant occupation during probe trial on day 7 in mice with indicated genotypes. Q1, target quadrant. Dashed line, random occupancy threshold. (d) is an image showing silver impregnation of cortical sections from 20-month-old Alz17.p38γ^{+/+} and Alz17.p38γ^{-/-}. Scale bar, 100 μm. (e) is an image showing immunofluorescence staining of phospho-S214 tau (pS214 tau) and neurofilament (NF) on cortical sections from Alz17.p38γ^{+/+} and Alz17.p38γ^{-/-} mice. Scale bar, 50 μm.

FIG. 8. Increased levels of neuronal p38γ and tau T205 phosphorylation in p38γ^{CA} mice does not increase toxicity of human tau. (a) is a schematic diagram: human wildtype tau transgenic mice (Alz17) were crossed with p38γ^{CA} (γCA) mice to achieve increased levels of active T205 tau kinase p38γ in neurons. (b) is an image showing immunofluorescence on histological sections from Alz17.p38γ^{CA}, Alz17 (10-month-old) for HA (p38γ^{CA}) and human tau. DAPI, nuclei. Scale bar, 10 μm. (n=4). (c) is an image showing immunoblots of lysates from crude synaptosome (CS) preparations from Alz17.p38γ^{+/+}, Alz17.p38γ^{-/-} and Alz17.p38γ^{CA} cortices for pT205 tau, PSD-95, p38γ, HA (p38γ^{CA}) and SNAP25. Whole brain lysate (W) and non-synaptosomal fraction (NS) from Alz17.p38γ^{+/+} cortex is shown as non-enriched control sample. #, non-specific band. (n=3-4). (d) is an image showing immunofluorescence on cortical histological sections from Alz17 and Alz17.p38γ^{CA} brains (10-month-old) for phospho-T205 tau, p38γ and human tau. Scale bar, 10 μm. (n=4). (e) is an image showing silver impregnation of cortical sections from 20-month-old Alz17 and Alz17. p38γ^{CA} mice. Scale bar, 100 μm. (n=4). (f-h) show memory/cognitive performance assessed using Morris water maze in 10-month-old Alz17, Alz17. p38γ^{CA} mice as well as p38γ^{CA} and non-transgenic control mice. (n=10). (f) is a graph showing Morris water maze acquisition phase: escape latency on days 1-7 in mice with indicated genotypes. (g) is a graph showing learning curve comparisons by linear regression slopes. (h) is a graph showing Morris water maze quadrant occupation during probe trial on day 8 in mice with indicated genotypes. Data are expressed as mean±S.E.M. **p<0.01 *p<0.05 ns, not significant. (2-way ANOVA for e; ANOVA for f).

FIG. 9. Amyloid burden and Control parameters for cognitive tests in APP23 mice with genome edited alleles for tauT205. (a) is an image showing immunofluorescence staining of phospho-T205 tau and human APP (6E10) in cortex of APP23.tauT205^{T/T} and APP23.tauT205^{A/A} mice. DAPI, nuclear marker. Scale bare, 50 μm. (n=6). (b) is a graph showing Survival curves of APP23.tauT205^{T/T} (n=40), APP23.tauT205^{T/A} (n=62) and APP23.tauT205^{T/A} (n=55). (c) is a graph showing Survival curves of APP23.tauT205^{T/T} (n=32), APP23.tauT205^{T/E} (n=71) and APP23.tauT205^{E/E} (n=38). (d) is a graph showing Morris water maze acquisition phase: visual cued trials in in APP23.tauT205^{T/T} and APP23.tauT205^{A/A} as well as tauT205^{T/T} and tauT205^{T/A} mice. (e) is a graph showing Morris water maze visual cued trials in in APP23.tauT205^{T/T} and APP23.tauT205^{E/E} as well as tauT205^{T/T} and tauT205^{E/E} mice. (f) is a graph showing Morris water maze retrieval tests: swim speed during probe trials in in APP23.tauT205^{T/T} and APP23.tauT205^{E/E} as well as tauT205^{T/T} and tauT205^{E/E} mice. Data are expressed as mean±S.E.M.

FIG. 10. Immunodetection of viral transgenes and control parameters for cognitive tests in APP23.tau^{-/-}.p38γ^{CA} with AAV expressing neuronal tauWT or tauT205A. (a) is images showing immunofluorescence staining for p38γ^{CA} (HA) and human tau on cortical sections from APP23.tau^{-/-}.p38γ^{CA} mice injected with indicated AAV. DAPI, nuclear marker. Scale bare, 50 μm. (n=5). (b) is images showing immunofluorescence staining of phospho-T205 tau and human APP (6E10) in cortex of APP23.tau^{-/-}.p38γ^{CA} mice injected with indicated AAV. DAPI, nuclear marker. Scale bare, 50 μm. (n=5). FIGS. 10(c-g) show Memory/cognitive performance assessed using Morris water maze in APP23.tau^{-/-}.p38γ^{CA} mice with neuronal expression of tauWT or tauT205A. (n as indicated in label legend). (c, d, e) are graphs showing Acquisition phase: escape latencies of all experimental groups (c) , non-transgenic tau^{-/-} and tau^{-/-}.p38γ^{CA} mice (d) and APP23.tau^{-/-} and APP23.tau^{-/-}.p38γ^{CA} mice (e) with indicated AAV-mediated expression of tauWT or tauT205A. (f) is a graph showing Acquisition phase : learning curve comparisons of all experimental groups. (g) is a graph showing Morris water maze: probe trial results on day 8. Q1, target quadrant. Dashed line, random occupancy threshold. Data are expressed as mean±S.E.M. **p<0.01 *p<0.05 ns, not significant. (2-way ANOVA for c, d, e; ANOVA for f, g).

FIG. 11 is a graph showing the time spent in open arms of a mouse maze by wild-type mice, Tau58 mutant mice expressing GFP, or Tau58 mutant mice expressing constitutively active p38γ (Cap38γ).

FIG. 12 is a graph showing the time spent in closed arms of a mouse maze, and how many closed arm entries were made, by wild-type mice, Tau58 mutant mice expressing GFP, or Tau58 mutant mice expressing constitutively active p38γ (Cap38γ).

FIG. 13 is a graph showing time spent in the centre of a mouse maze, and distance travelled by mice in the maze, by wild-type mice, Tau58 mutant mice expressing GFP, or Tau58 mutant mice expressing constitutively active p38γ (Cap38γ).

FIG. 14 shows the amino acid sequence of mature wild-type human p38γ.

FIG. 15 shows an example of the nucleotide sequence encoding mature human p38γ.

FIG. 16 shows the amino acid sequence of an example of a constitutively active mutant of p38γ (D179A) (p38γ^{CA}).

FIG. 17 shows the amino acid sequence of mature wild-type full length human tau. The amino acid sequence SSPGSPGTPGSRSR within the tau is in bold, and T205 and S422 are underlined.

FIG. 18 shows the amino acid sequence of example of a phosphomimetic of tau in which threonine at position 205 of wild-type tau is changed to glutamic acid (T205E).

FIG. 19 is an image of Western blots of brain extracts from AAV-p38g^{CA} and control (AAV-GFP) treated TAU58/2 mice probed with antibodies as indicated.

### DETAILED DESCRIPTION

The present invention relates to a method of treating tauopathies. A tauopathy is a condition associated with aggregation of tau protein in neurons of the brain of a subject, and is typically associated with neurofibrillary deposits, such as neurofibrillary tangles formed of tau protein. It is believed that in tauopathies, tau protein aggregates over time resulting in formation of tau-containing neurofibrillary deposits which ultimately cause neuronal death. Typically, the tauopathy is a neurodegenerative disease associated with cognitive decline. Examples of tauopathies include Alzheimer's disease, frontotemporal lobar dementia, corticobasal degeneration, progressive supranuclear palsy, primary age-related tauopathy, chronic traumatic encephalopathy, frontotemporal dementia with parkinsonism linked to chromosome 17, Pick's disease, globular glial tauopathy, Parkinson's disease.

During the early stages of Alzheimer's disease (AD) and other neurodegenerative diseases, excitotoxicity of neurons is believed to be caused through stimulation of tau-dependent signalling complexes, such as PSD-95/tau/FYN receptor complexes. The inventors have shown previously that phosphorylation of tau at specific sites causes disruption of PSD-95/tau/FYN receptor complexes, thereby preventing excitotoxicity and further development of neurodegenerative conditions mediated by PSD-95/tau/FYN signalling complexes.

However, in advanced AD and other tauopathies, tau toxicity is independent of signalling through tau-dependent signalling complexes, such as PTD-95/tau/FYN receptor complexes, and neuronal toxicity and death is thought to be mediated by aggregated hyperphosphorylated tau. Prior to the present invention, it was believed that advanced AD and other tauopathies were not treatable because the effects of tau aggregation could not be halted or reversed once tau aggregation had occurred, and consequently neuronal damage and death was inevitable once tau aggregation had occurred.

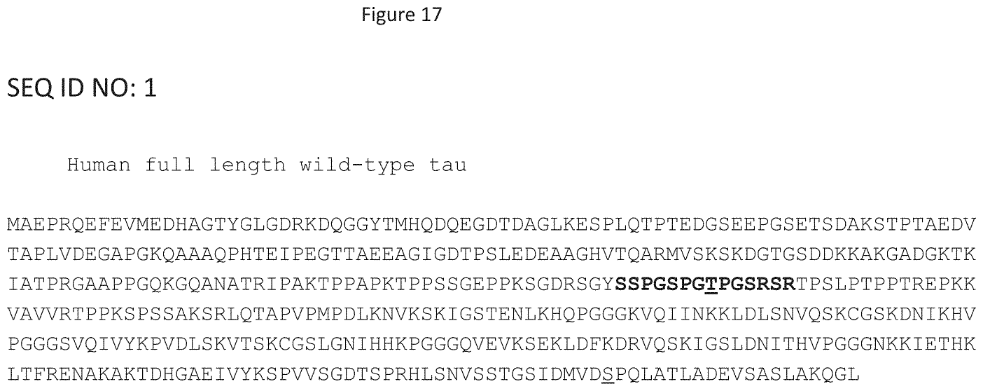

The inventors have now found that promoting phosphorylation of threonine in the sequence SSPGSPGTPGSRSR (SEQ ID NO: 7) of tau, such as the threonine residue at position 205 of the longest human isoform of tau (T205), or mutating wild-type tau to express a phosphomimetic of tau which has been phosphorylated at T205, in neurons of a subject, results in an improvement of cognitive function in tauopathies. The amino acid numbering used herein for tau is based on the amino acid numbering of the longest human isoform of tau with 441 amino acids, commonly referred to as 2N4R tau (and also referred to herein as full-length human tau). The amino acid sequence of the longest human isoform of tau (2N4R) is shown in FIG. 17 and represented by SEQ ID NO: 1. The amino acid numbering for tau is based on the full length human tau isoform comprising 441 amino acids (SEQ ID NO: 1).

As described in the Examples, the inventors have found that phosphorylation of human wild-type tau at position 205 (threonine) can improve cognitive function in a mouse model of advanced AD, and in some cases, can reverse the effects of tauopathy.

As further described in the Examples, the inventors have found that the symptoms of tauopathy can be reduced or reversed even when symptoms of tauopathy are due solely to aggregation of tau. In this regard, the inventors have shown that the effects resulting from tau aggregation can be reversed or reduced by phosphorylation of T205 in a mouse model in which tau aggregation is the only factor contributing to disease progression.

Accordingly, in one aspect there is provided a method of treating or preventing a tauopathy in a subject, comprising administering to the subject an effective amount of an agent which:

(a) promotes phosphorylation of tau at threonine in the amino acid sequence SSPGSPGTPGSRSR of tau, such as at threonine at position 205 of full-length human tau; and/or

(b) introduces a phosphomimetic of a phosphorylated tau, wherein the phosphorylated tau is tau that has been phosphorylated at threonine in the amino acid sequence SSPGSPGTPGSRSR of the tau, such as at threonine at position 205 of full-length human tau.

In another aspect, there is provided a method of improving cognitive ability in a subject suffering from cognitive impairment associated with a tauopathy, comprising administering to the subject an effective amount of an agent which:

(a) promotes phosphorylation of tau at threonine in the sequence SSPGSPGTPGSRSR of tau, such as at threonine at position 205 of full-length human tau; and/or

(b) introduces a phosphomimetic of a phosphorylated tau, wherein the phosphorylated tau is tau that has been phosphorylated at a threonine in the sequence SSPGSPGTPGSRSR of the tau, such as at threonine at position 205 of full-length human tau.

Cognitive ability is the ability of the brain to process, retrieve, and/or store information. An example of a cognitive ability is memory. One embodiment provides a method of improving memory in a subject suffering from cognitive impairment associated with a tauopathy, comprising administering to the subject an effective amount of an agent which:

(a) promotes phosphorylation of tau at threonine in the sequence SSPGSPGTPGSRSR of tau, such as at threonine at position 205 of full-length human tau; and/or

(b) introduces a phosphomimetic of a tau protein that has been phosphorylated at a threonine in the sequence SSPGSPGTPGSRSR of the tau, such as at threonine at position 205 of full length human tau.

An improvement in cognitive ability is an improvement or increase or enhancement in a subject's cognitive ability relative to the cognitive ability of the subject prior to treatment with the method described herein. An improvement in memory is an improvement or increase or enhancement in a subject's memory relative to the memory of the subject prior to treatment with the method described herein.

As described in the Examples, the inventors have further found that the level of phosphorylated serine 422 (pS422) in insoluble or aggregated tau is reduced in mice treated with activated p38γ (which promotes phosphorylation of T205 in full length human tau) relative to untreated mice in a mouse model of tauopathy.

Accordingly, one aspect provides a method of reducing phosphorylation of serine at position 422 of human tau in a subject suffering from a tauopathy, the method comprising administering to the subject an effective amount of an agent which:

(a) promotes phosphorylation of tau at threonine in the sequence SSPGSPGTPGSRSR of the tau, such as at threonine at position 205 of full-length human tau; and/or

(b) introduce a phosphomimetic of a phosphorylated tau, wherein the phosphorylated tau is tau that has been phosphorylated at threonine in the sequence SSPGSPGTPGSRSR of the tau, such as at threonine at position 205 of full-length human tau.

Another aspect provides a method of treating or preventing a disease or condition associated with phosphorylation of serine at position 422 of human tau in a subject, the method comprising administering to the subject an effective amount of an agent which:

(a) promotes phosphorylation of tau at threonine in the sequence SSPGSPGTPGSRSR of the tau, such as at threonine at position 205 of full-length human tau; and/or

(b) introduce a phosphomimetic of a phosphorylated tau, wherein the phosphorylated tau is tau that has been phosphorylated at threonine in the sequence SSPGSPGTPGSRSR of the tau, such as at threonine at position 205 of full-length human tau.

Phosphorylation of serine at position 422 of human tau is associated with neurofibrillary tangle formation.

Accordingly, another aspect provides a method of reducing or preventing neurofibrillary tangles in neurons of a subject, comprising administering an effective amount of an agent which:

(a) promote phosphorylation of tau at threonine in the sequence SSPGSPGTPGSRSR of the tau, such as at threonine at position 205 of full-length human tau; and/or

(b) introduce a phosphomimetic of a phosphorylated tau, wherein the phosphorylated tau is tau that has been phosphorylated at threonine in the sequence SSPGSPGTPGSRSR of the tau, such as at threonine at position 205 of full-length human tau.

In one embodiment, the method comprises administering the subject an effective amount of an agent which promotes phosphorylation of tau at threonine in the amino acid sequence SSPGSPGTPGSRSR of tau. In one embodiment, the threonine in the sequence SSPGSPGTPGSRSR of the tau is threonine at position 205 of full-length human tau (T205).

In one embodiment, the method comprises administering an effective amount of an agent which promotes phosphorylation of tau at threonine in the amino acid sequence SSPGSPGTPGSRSR of the tau, such as at threonine at position 205 of full-length human tau, in neurons of the subject, typically neurons of the brain of the subject.

In one embodiment, the subject is treated by administering an effective amount of an agent that elevates tau that has been phosphorylated at T205.

In one embodiment, the subject is treated by administering an agent which causes expression of a phosphomimetic of Tau pT205.

Tau that has been phosphorylated at a threonine at position 205 of full-length human tau is also referred to herein as tau pT205.

In one embodiment, the subject is treated by administering an effective amount of an agent which converts a gene or genes encoding wild-type tau, typically endogenous wild-type tau, to a gene encoding a phosphomimetic of tau pT205. In one embodiment, the phosphomimetic of tau pT205 is a tau which comprises the amino acid sequence SSPGSPGXPGSRSR (SEQ ID NO: 8), wherein X is E or D. In one embodiment, the phosphomimetic of tau pT205 is T205E or T205D.

As used herein, a phosphomimetic of phosphorylated tau is a variant of tau which functions in a manner that is the same as, or substantially the same as, that of the phosphorylated wild-type tau. Thus, a phosphomimetic of pT205 is a variant of tau that exhibits the same or similar effect to that of full-length wild-type tau that is phosphorylated ay threonine at position 205. As used herein, a variant of tau is tau protein comprising one or more amino acid substitutions, insertions or deletions, of the wild-type tau, typically the full-length wild-type tau (e.g., SEQ ID NO: 1).

It will be appreciated that a phosphomimetic of tau pT205 does not necessarily contain a mutation at T205, and may contain a substitution, deletion or insertion of one or more amino acids residues at other sites of tau which results in the mutated tau having the same or a similar effect as T205E.

The agent may comprise, for example, a nucleic acid, a nucleic acid analogue, a protein, a peptide, or a small molecule, or combinations thereof. Typically, administration of the agent introduces the agent into neurons of the subject. More typically, administration of the agent introduces the agent into neurons of the brain of the subject.

In some embodiments, the agent comprises a nucleic acid which is introduced into neurons of the subject. In some embodiments, the nucleic acid is transcribed in the neurons. In some embodiments, the nucleic acid is transcribed and translated in the neurons. In some embodiments, the nucleic acid comprises DNA. In some embodiments, the nucleic acid comprises RNA.

In some embodiments, the agent can cross the blood-brain barrier, or can be formulated to cross the blood-brain barrier.

In one embodiment, the tauopathy is Alzheimer's disease mediated by tau aggregation.

In one embodiment, the tauopathy is frontotemporal lobar dementia mediated by tau aggregation.

In one embodiment, the tauopathy is corticobasal degeneration.

In one embodiment, the tauopathy is progressive supranuclear palsy.

In one embodiment, the tauopathy is primary age-related tauopathy.

In one embodiment, the tauopathy is chronic traumatic encephalopathy.

In one embodiment, the tauopathy is frontotemporal dementia with parkinsonism linked to chromosome 17.

In one embodiment, the tauopathy is Pick's disease.

In one embodiment, the tauopathy is globular glial tauopathy.

In one embodiment, the tauopathy is Parkinson's disease.

As used herein, a “subject” is a mammal. The mammal can be a human, non-human primate, sheep, mouse, rat, dog, cat, horse, cow, pig, or any other mammals which can suffer from a tauopathy. Typically, the subject is a human.

In one embodiment, the subject is treated by administering an effective amount of an agent that elevates p38γ activity, or activity of a variant of p38γ, in neurons of the subject. p38γ, also known as ERK6, SAPK3 and MAPK12, is a mitogen activated protein kinase (MAP Kinase). In one embodiment, the p38γ is from a mammal. For example, the p38γ may be from a human, mouse, dog, cat, pig, cow, rat, non-human primate, goat, sheep. Typically, the p38γ is human p38γ. Wild-type p38γ is activated through phosphorylation of tyrosine and threonine residues in the motif TGY. Wild-type p38γ phosphorylates tau following activation. Activation of p38γ is carried out by the MAP kinase kinases MKK3 and MKK6, which are in turn activated upon phosphorylation by the MAPK kinase MAP3K.

As described in the Examples, the inventors have found that phosphorylation of wild-type human tau at T205 by p38γ results in improved cognitive function in a mouse model of advanced Alzheimer's disease. The inventors have shown that by introducing p38γ, or a constitutively active variant of p38γ, into neurons of mice suffering from advanced AD, memory is improved.

One embodiment provides a method of treating or preventing a tauopathy in a subject, comprising administering an effective amount of an agent which elevates p38γ activity, or the activity of a variant of p38γ, in neurons of the subject.

One embodiment provides a method of improving cognitive ability in a subject suffering from cognitive impairment associated with a tauopathy, comprising administering an effective amount of an agent which elevates p38γ activity, or the activity of a variant of p38γ, in neurons of the subject.

One embodiment provides a method of reducing phosphorylation of serine at position 422 of human tau in a subject suffering from a tauopathy, comprising administering an effective amount of an agent which elevates p38γ activity, or the activity of a variant of p38γ, in neurons of the subject.

One embodiment provides a method of treating or preventing a tauopathy associated with phosphorylation of serine at position 422 of human tau in a subject, comprising administering an effective amount of an agent which elevates p38γ activity, or the activity of a variant of p38γ, in neurons of the subject.

One embodiment provides a method of reducing or preventing tau aggregation in neurons of a subject, comprising administering an effective amount of an agent which elevates p38γ activity, or the activity of a variant of p38γ, in neurons of the subject.

One embodiment provides a method of reducing or preventing neurofibrillary tangles in neurons of a subject, comprising administering an effective amount of an agent which elevates p38γ activity, or the activity of a variant of p38γ, in neurons of the subject.

An agent that elevates p38γ activity, or the activity of a variant of p38γ, in a neuron may be an agent that: (a) elevates the amount of p38γ, typically the amount of active p38γ, in the neuron; and/or (b) elevates the amount of a variant of p38γ, typically the amount of an active variant of p38γ, in the neuron; and/or (c) elevates the amount of p38γ activation in the neuron; and/or (d) elevates the amount of activation of the variant of p38γ in the neuron, if the variant is not an active variant. As used herein, “p38γ activity” is an activity of activated p38γ that phosphorylates tau at threonine in the sequence SSPGSPGTPGSRSR of tau, and in the case of wild-type full length human tau, phosphorylates Tau at T205. The “activity of a variant of p38γ ” refers to an activity of a variant of p38γ which is the same as, or substantially similar to, p38γ activity. The variant of p38γ may be capable of p38γ activity without activation (for example, an active variant, such as a constitutively active variant), or may exhibit p38γ activity following activation. p38γ activity is elevated in a neuron when the amount of p38γ activity in the neuron after treatment is increased relative to the amount of p38γ activity in the neuron prior to treatment. The activity of a variant of p38γ is elevated in a neuron when the amount of activity of the variant in the neuron after treatment is increased relative to the amount of activity of the variant in the neuron prior to treatment. The p38γ activity, or the activity of a variant of p38γ, may be elevated by administering an effective amount of an agent which elevates:

(a) the amount of endogenous p38γ in the neurons, such as increasing expression (transcription and/or translation) of endogenous p38γ; and/or

(b) the amount of exogenous p38γ in the neurons; and/or

(c) the amount of a variant of p38γ in the neurons; and/or

(d) the activation of endogenous p38γ, exogenous p38γ and/or a variant of p38γ in the neurons.

In one embodiment, the p38γ activity, or the activity of a variant of p38γ, is elevated by administering an effective amount of an agent which elevates the amount of exogenous p38γ, or a variant thereof, in neurons. The amount of exogenous p38γ, or a variant thereof, may be elevated by introducing into neurons p38γ, or a variant thereof, or by introducing into neurons a nucleic acid capable of expressing p38γ, or a variant thereof.

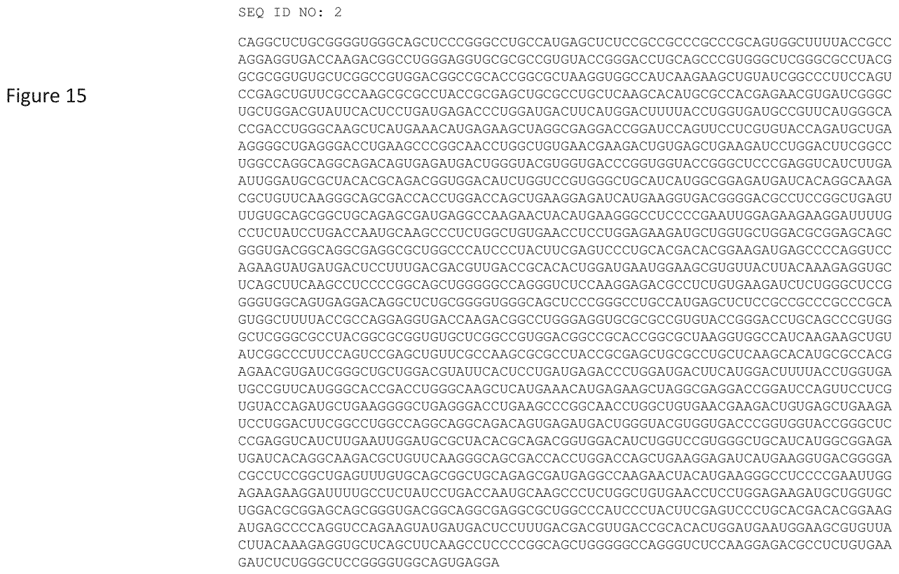

Thus, in one embodiment, the agent which elevates p38γ activity, or the activity of a variant of p38γ, in neurons of the subject, may comprise the p38γ protein or a variant thereof, or a nucleic acid that is capable of expressing p38γ or a variant thereof, in neurons of the subject. The nucleic acid sequence encoding full-length wild-type human p38γ and the amino acid sequence of full-length wild-type human p38γ used in the Examples described herein is shown in FIG. 15 (SEQ ID NO: 2) and 14 (SEQ ID NO: 3). Naturally occurring isoforms and variants of human p38γ are also known (e.g. Genbank accession nos. NP_001290181, CR456515). It is envisaged that natural isoforms or variants of p38γ that phosphorylate tau at T205 could be used in the methods described herein.

In one embodiment, the agent which elevates p38γ activity, or the activity of a variant of p38γ, comprises a nucleic acid that encodes p38γ or a variant thereof. Those skilled in the art will be able to determine the appropriate nucleic acid sequence which encodes the amino acid sequence of the p38γ or variant thereof. For example, a nucleic acid which encodes p38γ, may comprise a nucleic acid sequence that is in the range of from about 60% to 100% identical to the wild-type coding sequence of human p38γ (SEQ ID NO: 3). For example, the nucleic acid encoding p38γ may have a sequence that has at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the wild-type coding sequence of p38γ using one of the alignment programs described herein using standard parameters. Those skilled in the art will recognize that these values can be appropriately adjusted to determine corresponding identity of proteins encoded by a nucleotide sequence by taking into account codon degeneracy, reading frame positioning, and the like.

In one embodiment, the agent which elevates p38γ activity, or the activity of a variant of p38γ, comprises a variant of p38γ. In one embodiment, the agent which elevates p38γ activity, or the activity of a variant of p38γ, comprises a nucleic acid that encodes a variant of p38γ. As used herein, a variant of p38γ is a protein which differs from the wild-type human p38γ protein by one or more amino acid substitutions, additions or deletion, and which is capable of phosphorylating tau at threonine in the sequence SSPGSPGTPSRSR, such as phosphorylating wild-type human tau at threonine residue T205. The variant of p38γ phosphorylates wild-type human tau at residue T205. In one embodiment, the variant of p38γ comprises an amino acid sequence that is at least 60%, 65%, 70%, 75%, 80%, 85%, 90%, 95%, or 99% identical to the amino acid sequence of wild-type human p38γ. In one embodiment, the variant of p38γ comprises an amino acid sequence that is at least 60%, 65%, 70%, 75%, 80%, 85%, 90%, 95%, or 99% identical to the amino acid sequence represented by SEQ ID NO: 3.

As used herein, “% identity” with reference to a polypeptide, or “% identical to the amino acid sequence” of a polypeptide, refers to the percentage of residues in the two sequences that are the same when aligned for maximum correspondence over a specified comparison window, as measured by sequence comparison algorithms or by visual inspection.

Sequence comparison algorithms for determining % identity between two polypeptides are known in the art. Examples of such algorithms are the algorithm of Myers and Miller (1988); the local homology algorithm of Smith et al. (1981); the homology alignment algorithm of Needleman and Wunsch (1970); the search-for-similarity-method of Pearson and Lipman (1988); and the algorithm of Karlin and Altschul (1990), modified as in Karlin and Altschul (1993). Computer implementations of these algorithms for determining % identity between two polypeptides include, for example: CLUSTAL (available from Intelligenetics, Mountain View, Calif.) (Pearson et al. (1994)).; the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA).

In some embodiments, the variant of p38γ may comprise a part of p38γ.

In some embodiments, the variant of p38γ may comprise a part of p38γ but otherwise differ from the wild-type p38γ. In this regard, the inventors envisage that variants of p38γ may include chimeric p38γ protein in which an interaction motif of p38γ is fused to portions of other kinases, such as MAP kinase or other serine/threonine kinases, or variants of other kinases that carry mutations to modify their activity. For example, the variant of p38γ may comprise a carboxy terminal portion of p38γ fused to the N-terminal portion of a kinase selected from the group consisting of p38α, p38β and p38δ, or variants of p38α, p38β and p38δ that carry mutations that modify their activity. In one embodiment, the variant of p38γ is a chimeric p38γ. In various embodiments, the chimeric p38γ comprises an amino acid sequence selected form the group consisting of: ETPL (SEQ ID NO: 9), KETPL (SEQ ID NO: 10), SKETPL (SEQ ID NO: 11), VSKETPL (SEQ ID NO: 12), RVSKETPL (SEQ ID NO: 13), ARVSKETPL (SEQ ID NO: 14), GARVSKETPL (SEQ ID NO: 15), LGARVSKETPL (SEQ ID NO: 16), QLGARVSKETPL (SEQ ID NO: 17), RQLGARVSKETPL (SEQ ID NO: 18), PRQLGARVSKETPL (SEQ ID NO: 19), PPRQLGARVSKETPL (SEQ ID NO: 20), KPPRQLGARVSKETPL (SEQ ID NO: 21), FKPPRQLGARVSKETPL (SEQ ID NO: 22), SFKPPRQLGARVSKETPL (SEQ ID NO: 23), LSFKPPRQLGARVSKETPL (SEQ ID NO: 24), VLSFKPPRQLGARVSKETPL (SEQ ID NO: 25), EVLSFKPPRQLGARVSKETPL (SEQ ID NO: 26), KEVLSFKPPRQLGARVSKETPL (SEQ ID NO: 27), YKEVLSFKPPRQLGARVSKETPL (SEQ ID NO: 28), TYKEVLSFKPPRQLGARVSKETPL (SEQ ID NO: 29), VTYKEVLSFKPPRQLGARVSKETPL (SEQ ID NO: 30), RVTYKEVLSFKPPRQLGARVSKETPL (SEQ ID NO: 31), KRVTYKEVLSFKPPRQLGARVSKETPL (SEQ ID NO: 32), ETAL (SEQ ID NO: 33), KETAL (SEQ ID NO: 34), PKETAL (SEQ ID NO: 35), VPKETAL (SEQ ID NO: 36), RVPKETAL (SEQ ID NO: 37), ARVPKETAL (SEQ ID NO: 38), GARVPKETAL (SEQ ID NO: 39), LGARVPKETAL (SEQ ID NO: 40), QLGARVPKETAL (SEQ ID NO: 41), RQLGARVPKETAL (SEQ ID NO: 42), PRQLGARVPKETAL (SEQ ID NO: 43), PPRQLGARVPKETAL (SEQ ID NO: 44), KPPRQLGARVPKETAL (SEQ ID NO: 45), FKPPRQLGARVPKETAL (SEQ ID NO: 46), SFKPPRQLGARVPKETAL (SEQ ID NO: 47), LSFKPPRQLGARVPKETAL (SEQ ID NO: 48), VLSFKPPRQLGARVPKETAL (SEQ ID NO: 49), EVLSFKPPRQLGARVPKETAL (SEQ ID NO: 50), KEVLSFKPPRQLGARVPKETAL (SEQ ID NO: 51), YKEVLSFKPPRQLGARVPKETAL (SEQ ID NO: 52), TYKEVLSFKPPRQLGARVPKETAL (SEQ ID NO: 53), VTYKEVLSFKPPRQLGARVPKETAL (SEQ ID NO: 54), RVTYKEVLSFKPPRQLGARVPKETAL (SEQ ID NO: 55), and KRVTYKEVLSFKPPRQLGARVPKETAL (SEQ ID NO: 56).

In one embodiment, the variant of p38γ is an active variant of p38γ. An active variant of p38γ is a variant which does not require activation by the MAP kinase kinases MKK3 and MKK6 in order to exhibit p38γ activity. In one embodiment, the active variant of p38γ is a constitutively active variant of p38γ. A constitutively active variant of p38γ is a variant of p38γ which is continuously active and therefore does not require activation by the MAP kinase kinases MKK3 and MKK6. Typically, a constitutively active variant comprises one or more amino acid substitutions which result in continuous activity. In various embodiments, the constitutively active variant of p38g comprises the amino acid sequence:

(a)
(SEQ ID NO: 57)
ARQAASEMTGY;
(b)
(SEQ ID NO: 58)
LARQAASEMTGYV
(c)
(SEQ ID NO: 59)
DFGLARQAASEMTGYVVTRW
(d)
(SEQ ID NO: 60)
VNEDCELKILDFGLARQAASEMTGYVVTRWYRAPEVILNW
(e)
(SEQ ID NO: 61)
HRDLKPGNLAVNEDCELKILDFGLARQAASEMTGYVVTRWYRAPEVILN
WMRYTQTVDIW;
(f)
(SEQ ID NO: 62)
LRYIHAAGIIHRDLKPGNLAVNEDCELKILDFGLARQAASEMTGYVVTR
WYRAPEVILNWMRYTQTVDIWSVGCIMAEMI;
(g)
(SEQ ID NO: 63)
QFLVYQMLKGLRYIHAAGIIHRDLKPGNLAVNEDCELKILDFGLARQAA
SEMTGYVVTRWYRAPEVILNWMRYTQTVDIWSVGCIMAEMITGKTLFKG
SD;
(h)
(SEQ ID NO: 64)
KHEKLGEDRIQFLVYQMLKGLRYIHAAGIIHRDLKPGNLAVNEDCELKI
LDFGLARQAASEMTGYVVTRWYRAPEVILNWMRYTQTVDIWSVGCIMAE
MITGKTLFKGSDHLDQLKEIMK;
(i)
(SEQ ID NO: 65)
FMGTDLGKLMKHEKLGEDRIQFLVYQMLKGLRYIHAAGIIHRDLKPGNL
AVNEDCELKILDFGLARQAASEMTGYVVTRWYRAPEVILNWMRYTQTVD
IWSVGCIMAEMITGKTLFKGSDHLDQLKEIMKVTGTPPAEFV;
j)
(SEQ ID NO: 66)
DFTDFYLVMPFMGTDLGKLMKHEKLGEDRIQFLVYQMLKGLRYIHAAGI
IHRDLKPGNLAVNEDCELKILDFGLARQAASEMTGYVVTRWYRAPEVIL
NWMRYTQTVDIWSVGCIMAEMITGKTLFKGSDHLDQLKEIMKVTGTPPA
EFVQRLQSDEAKN;
k)
(SEQ ID NO: 67)
DVFTPDETLDDFTDFYLVMPFMGTDLGKLMKHEKLGEDRIQFLVYQMLK
GLRYIHAAGIIHRDLKPGNLAVNEDCELKILDFGLARQAASEMTGYVVT
RWYRAPEVILNWMRYTQTVDIWSVGCIMAEMITGKTLFKGSDHLDQLKE
IMKVTGTPPAEFVQRLQSDEAKNYMKGLPELEK;
l)
(SEQ ID NO: 68)
MRHENVIGLLDVFTPDETLDDFTDFYLVMPFMGTDLGKLMKHEKLGEDR
IQFLVYQMLKGLRYIHAAGIIHRDLKPGNLAVNEDCELKILDFGLARQA
ASEMTGYVVTRWYRAPEVILNWMRYTQTVDIWSVGCIMAEMITGKTLFK
GSDHLDQLKEIMKVTGTPPAEFVQRLQSDEAKNYMKGLPELEKKDFASI
LTNA.

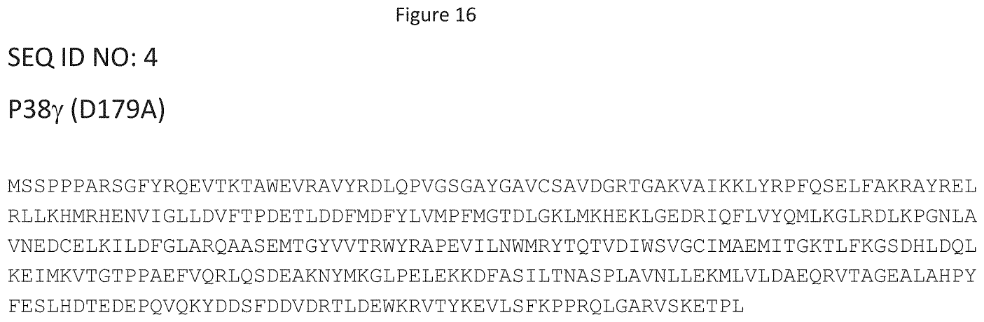

In one embodiment, the constitutively active variant of p38γ comprises the amino acid substitution of D179A of wild-type human p38γ. The amino acid sequence of an example of a constitutively active variant of p38γ is shown in FIG. 16 (SEQ ID NO: 4).

In one embodiment, there is provided a method of treating or preventing a tauopathy in a subject, comprising administering an effective amount of a nucleic acid which expresses p38γ or a variant thereof, typically a constitutively active variant of p38γ, in neurons of the subject. Typically, the tauopathy is associated with phosphorylation of serine at position 422 of human tau.

In one embodiment, there is provided a method of improving cognitive ability, such as memory, in a subject suffering from cognitive impairment associated with a tauopathy, comprising administering an effective amount of a nucleic acid which expresses p38γ or a variant thereof, typically a constitutively active variant of p38γ, in neurons of the subject. Typically, the tauopathy is associated with phosphorylation of serine at position 422 of human tau.

In one embodiment, there is provided a method of treating advanced Alzheimer's disease in a subject, comprising administering an effective amount of a nucleic acid which expresses p38γ or a variant thereof, typically a constitutively active variant of p38γ, in neurons of the subject. Typically, the advanced Alzheimer's disease is associated with phosphorylation of serine at position 422 of human tau.

In one embodiment, there is provided a method of treating advanced frontotemporal dementia in a subject, comprising administering an effective amount of a nucleic acid which expresses p38γ or a variant thereof, typically a constitutively active variant of p38γ, in neurons of the subject. Typically, the frontotemporal dementia is associated with phosphorylation of serine at position 422 of human tau.

In one embodiment, there is provided a method of reducing phosphorylation of serine at position 422 of human tau in a subject suffering from a tauopathy, comprising administering an effective amount of a nucleic acid which expresses p38γ or a variant thereof, typically a constitutively active variant of p38γ, in neurons of the subject.

In one embodiment, there is provided a method of treating a or preventing tauopathy associated with phosphorylation of serine at position 422 of human tau in a subject, comprising administering an effective amount of a nucleic acid which expresses p38γ or a variant thereof, typically a constitutively active variant of p38γ, in neurons of the subject.

Another embodiment provides method of reducing or preventing tau aggregation in neurons of a subject, comprising administering an effective amount of a nucleic acid which expresses p38γ or a variant thereof, typically a constitutively active variant of p38γ, in neurons of the subject.

Another embodiment provides a method of reducing or preventing neurofibrillary tangles in neurons of a subject, comprising administering an effective amount of a nucleic acid which expresses p38γ or a variant thereof, typically a constitutively active variant of p38γ, in neurons of the subject.

In another embodiment, the subject is treated by administering an agent that introduces into neurons of the subject a phosphomimetic of tau pT205. In one form, an agent that introduces a phosphomimetic of tau pT205 is an agent that introduces a phosphomimetic mutation into wild-type tau, typically into endogenous wild-type tau. As used herein, a phosphomimetic of tau pT205 is a variant of tau comprising one or more amino acid substitutions, insertions or deletions, and which functions in a manner that is the same as, or substantially the same as, that of unsubstituted wild-type human tau following phosphorylation of the unsubstituted tau at threonine 205. In one embodiment, a phosphomimetic comprises a phosphomimetic substitution.

As described in the Examples, the inventors have shown that expression of a T205E variant of tau in neurons improved cognitive function and survival in an AD mouse model. The T205E variant of tau is a phosphomimetic of tau phosphorylated at T205 (pT205). A phosphomimetic substitution is an amino acid substitution in a protein which results in the protein functioning in a manner which is the same as, or substantially the same as, the unsubstituted protein following phosphorylation of the unsubstituted protein. A phosphomimetic substitution of phosphorylated tau is an amino acid substitution at a site of tau which results in a tau protein that functions in the same, or substantially the same, manner to the wild-type tau following phosphorylation of the wild-type tau at a particular site.

In one embodiment, the method comprises treating the subject to introduce a phosphomimetic of tau comprising a phosphomimetic substitution of tau at T205.

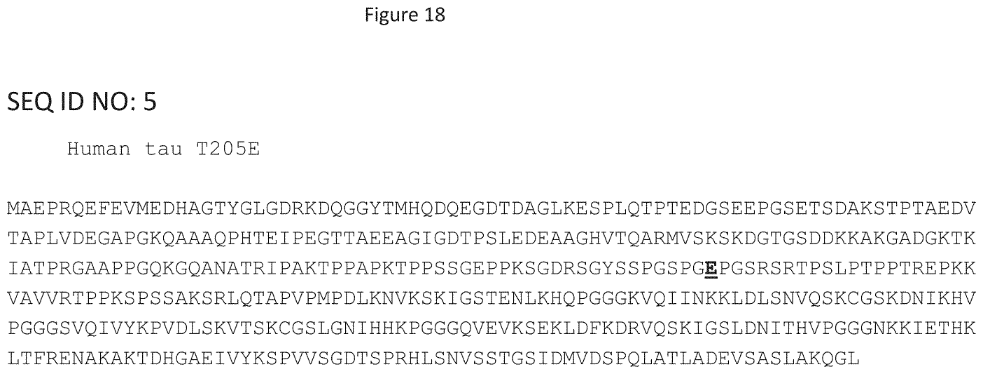

In one embodiment, the phosphomimetic substitution of tau is threonine to glutamic acid or aspartic acid at position 205 of tau (T205E or T205D), with amino acid numbering based on the longest human tau isoform comprising 441 amino acids. The amino acid sequence of full-length wild-type human tau (SEQ ID NO: 1) and tau T205E (SEQ ID NO: 5) is shown in FIGS. 17 and 18.

Typically, the variant of tau is a variant of human tau. In other embodiments, the variant of tau may be a variant of tau from a non-human mammal. For example, the variant of tau may be a variant of tau from a mouse, dog, cat, pig, cow, rat, non-human primate, goat, or sheep.

In one embodiment, there is provided a method of treating or preventing a tauopathy in a subject, comprising administering a nucleic acid comprising a nucleotide sequence which results in production of a tau which differs from wild-type human tau in an amino acid substitution of threonine to glutamic acid or aspartic acid at position 205 (T205E or T205D), in neurons of the subject.

In one embodiment, there is provided a method of improving cognitive ability in a subject suffering from cognitive impairment associated with a tauopathy, comprising administering a nucleic acid comprising a nucleotide sequence which results in production of a tau which differs from wild-type human tau in an amino acid substitution of threonine to glutamic acid or aspartic acid at position 205 (T205E or T205D), in neurons of the subject.

In one embodiment, there is provided a method of treating advanced Alzheimer's disease in a subject, comprising administering a nucleic acid comprising a nucleotide sequence which when expressed results in production of a tau which differs from wild-type human tau in an amino acid substitution of threonine to glutamic acid or aspartic acid at position 205 (T205E or T205D), in neurons of the subject.

In one embodiment, there is provided a method of treating advanced frontotemporal dementia in a subject, comprising administering a nucleic acid comprising a nucleotide sequence which when expressed results in production of a tau which differs from wild-type tau in an amino acid substitution of threonine to glutamic acid or aspartic acid at position 205 (T205E or T205D), in neurons of the subject.

In one embodiment, there is provided a method of reducing phosphorylation of serine at position 422 of human tau in a subject suffering from a tauopathy, comprising administering a nucleic acid comprising a nucleotide sequence which when expressed results in production of a tau which differs from wild-type tau in an amino acid substitution of threonine to glutamic acid or aspartic acid at position 205 (T205E or T205D), in neurons of the subject.

In one embodiment, there is provided a method of treating or preventing a tauopathy associated with phosphorylation of serine at position 422 of human tau in a subject, comprising administering a nucleic acid comprising a nucleotide sequence which when expressed results in production of a tau which differs from wild-type tau in an amino acid substitution of threonine to glutamic acid or aspartic acid at position 205 (T205E or T205D), in neurons of the subject.

Another embodiment provides method of reducing or preventing tau aggregation in neurons of a subject, comprising administering a nucleic acid comprising a nucleotide sequence which when expressed results in production of a tau which differs from wild-type tau in an amino acid substitution of threonine to glutamic acid or aspartic acid at position 205 (T205E or T205D), in neurons of the subject.

Another embodiment provides a method of reducing or preventing neurofibrillary tangles in neurons of a subject, comprising administering a nucleic acid comprising a nucleotide sequence which when expressed results in production of a tau which differs from wild-type tau in an amino acid substitution of threonine to glutamic acid or aspartic acid at position 205 (T205E or T205D), in neurons of the subject.

In one embodiment, the nucleic acid comprises a nucleotide sequence which encodes Tau T205E or T205D.

In some embodiments, the phosphomimetic of phosphorylated tau may be introduced into neurons by introducing a mutation into nucleotide sequence encoding wild-type tau in the neuron. Typically, the mutation is introduced into the endogenous tau gene in the neuron. Thus, in one embodiment, the nucleic acid is, or encodes, a gene editing system, or part of a gene editing system for introducing a phosphomimetic mutation into nucleotide sequence encoding wild-type tau, typically into the endogenous tau gene, in neurons of the subject. In on embodiment, the nucleic acid comprises a gene editing system, or part of a tau gene editing system, for introducing a phosphomimetic mutation into nucleotide sequence encoding wild-type tau, typically into the endogenous tau gene, in neurons of the subject. In one embodiment, the nucleic acid comprises nucleotide sequence that encodes a tau gene editing system, or part thereof, which introduces a mutation into the wild-type tau gene to cause expression of a phosphomimetic Tau. Typically, the phosphomimetic Tau is T205E or T205D. In one embodiment, the gene editing system is a CRISPR/Cas complex, typically CRISPR/Cas9 complex, which introduces into the tau gene a substitution of threonine for glutamic acid or aspartic acid at position 205 of human tau. Typically, the nucleic acid encoding the gene editing system, or part thereof, comprises a guide RNA, or a nucleic acid encoding a guide RNA (gRNA). Typically, the gRNA is a single guide RNA or a pair of guide RNAs.

As used herein, the term “tau gene” has the same meaning as “Mapt gene”.

In embodiments in which the agent comprises a nucleic acid that is capable of expressing p38γ or a variant thereof, or the variant of tau, or a gene editing system, in neurons of the subject, the nucleic acid sequence encoding p38γ or a variant thereof, or the variant of tau, or the gene editing system, is typically operably linked to regulatory sequence to direct expression of the p38γ, or a variant thereof, or the variant of tau, or the gene editing system, in the neurons of the subject. A nucleic acid that is capable of expressing p38γ or a variant thereof, or a variant of tau, or a gene editing system, in neurons of a subject may comprise an expression cassette comprising the coding sequence of p38γ or variant thereof, or the variant of tau, or encoding a gene editing system. An expression cassette is a nucleic acid comprising coding sequence and regulatory sequence which operate together to express a protein encoded by coding sequence in a cell. “Coding sequence” refers to a DNA or RNA sequence that codes for a specific amino acid sequence. It may constitute an “uninterrupted coding sequence”, i.e., lacking an intron, such as in a cDNA, or it may include one or more introns bounded by appropriate splice junctions.

The expression cassette typically includes regulatory sequences. A “regulatory sequence” is a nucleotide sequence located upstream (5′ non-coding sequences), within, or downstream (3′ non-coding sequences) of a coding sequence, and which influences the transcription, RNA processing or stability, or translation of the associated coding sequence. Regulatory sequences are known in the art and may include, for example, transcriptional regulatory sequences such as promoters, enhancers, translation leader sequences, introns, and polyadenylation signal sequences. The coding sequence is typically operably linked to a promoter. A promoter is a DNA region capable under certain conditions of binding RNA polymerase and initiating transcription of a coding sequence usually located downstream (in the 3′ direction) from the promoter. The coding sequence may also be operably linked to termination signals. The expression cassette may also include sequences required for proper translation of the coding sequence. The expression cassette including the coding sequence may be chimeric. A “chimeric” vector or expression cassette, as used herein, means a vector or cassette including nucleic acid sequences from at least two different species, or has a nucleic acid sequence from the same species that is linked or associated in a manner that does not occur in the “native” or wild type of the species. The coding sequence in the expression cassette may be under the control of a constitutive promoter or of a regulatable promoter that initiates transcription only in a particular tissue or cell type, or when the host cell is exposed to some particular stimulus. For example, in an expression cassette comprising a nucleic acid encoding p38γ, the coding sequence may be operably linked to a promoter which is not native to the p38γ gene, such as a promoter that expresses the coding sequence in, or is inducible in, neurons. In one embodiment, the promoter is a neural promoter. Examples of suitable neural promoters include synapsin (SYN), calcium/calmodulin-dependent protein kinase (CaMKII), tubulin alpha I (Ta1), neuron-specific enolase (NSE), platelet derived growth factor beta chain (PDGF), MfP, dox, GFAP, Preproenkephalin, dopamine β-hydroxylase (dβH), prolactin, chicken beta actin, prion protein, murine Thy1.2, myelin basic promoter, or any of the above combined with an enhancer, such as a partial cytomegaly virus promoter. Examples of other promoters which may be used to express nucleic acid sequence in neurons include, the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, synthetic promoters, hybrid promoters, and the like. Inducible or controllable promoters include, for example, promoters whose transcriptional activity is modified in the presence or absence of mifepristone, doxycycline, tetracycline or tamoxifen.

A nucleic acid encoding a protein (coding sequence) is operably linked to a regulatory sequence when it is arranged relative to the regulatory sequence to permit expression of the protein in a cell. For instance, a promoter is operatively linked to a coding region if the promoter helps initiate transcription of the coding sequence.

As used herein, “expression” of a nucleic acid sequence refers to the transcription and/or translation of a nucleic acid sequence comprising a coding sequence to produce the polypeptide encoded by the coding sequence, or comprising sequence encoding gene editing sequence such as CRISPR/Cas sequence.

In one embodiment, the agent is a vector. In such vectors, the nucleic acid sequence encoding p38γ or variant thereof, or the variant of tau, or a tau gene editing system, or an expression cassette comprising such sequences, is inserted into an appropriate vector sequence. The term “vector” refers to a nucleic acid suitable for transferring genes into a host cell, such as a neuron. The term “vector” includes plasmids, cosmids, naked DNA, viral vectors, etc. In one embodiment, the vector is a plasmid vector. A plasmid vector is a double stranded circular DNA molecule into which additional sequence may be inserted. The plasmid may be an expression vector. Plasmids and expression vectors are known in the art and described in, for example, Sambrook et al. Molecular Cloning: A Laboratory Manual, 4^{th} Ed. Vol. 1-3, Cold Spring Harbor, N.Y. (2012).

In some embodiments, the vector is a viral vector. Viral vectors comprise viral sequence which permits, depending on the viral vector, viral particle production and/or integration into the host cell genome and/or viral replication. Viral vectors which can be utilized with the methods and compositions described herein include any viral vector which is capable of introducing a nucleic acid into neurons, typically neurons of the brain. Examples of viral vectors include adenovirus vectors; lentiviral vectors; adeno-associated viral vectors; Rabiesvirus vectors; Herpes Simplex viral vectors; SV40; polyoma viral vectors; poxvirus vector.

In one embodiment, the viral vector is an adeno-associated viral (AAV) vector for packaging in an adeno-associated virus. In one embodiment, the AAV vector is a serotype selected from the group consisting of AAV1, AAV2, AAV3, AAV4, AAV5, AAV6, AAV6.2, AAV7, AAV8, AAV9, AAVrh10, AAVrh20, AAVrh39, AAVrh43, and AAVcy5 vector or variants thereof. In one embodiment, the viral vector is serotype AAV1, AAV9, AAVrh10 or AAVcy5. In one embodiment, the serotype of the AAV vector is AAV1. In another embodiment, the serotype of the AAV vector is AAV9. In another embodiment, the serotype of the AAV vector is AAVrh10. In another embodiment, the serotype of the AAV vector is AAVcy5. The use of recombinant AAV for introducing nucleic acids into cells is known in the art and described in, for example, US20160038613; Grieger and Samulski (2005) Adeno-associated virus as a gene therapy vector: vector development, production and clinical applications, Advances in Biochemical Engineering/Biotechnology 99: 119-145; Methods for the production of recombinant AAV are known in the art and described in, for example, Harasta et al (2015) Neuropsychopharmacology 40: 1969-1978. Example of adeno-associated viral vectors for expressing p38γ and p38γ^{CA} in neuronal cells is described in WO 2017/147654.

In another embodiment, the viral vector is a lentiviral vector. Methods for production and use of lentiviral vectors are known in the art and described in, for example, Naldini et al. (1996) In vivo gene delivery and stable transduction of nondividing cells by a lentiviral vector, Science, 272:263-267; Lois et al. (2002) Germline transmission and tissue-specific expression of transgenes delivered by lentiviral vectors, Science,295:868-872; Vogel et al (2004), A single lentivirus vector mediates doxycycline-regulated expression of transgenes in the brain. Hum Gene Ther. 2004;15(2):157-165.

Adenoviruses are also contemplated for use in delivery of nucleic acid agents. Thus, in another embodiment, the viral vector is an adenoviral vector. Adenoviral vectors are known in the art and described in, for example, Kozarsky and Wilson, Current Opinion in Genetics and Development 3:499-503 (1993); Southgate et al. (2008) Gene transfer into neural cells in vitro using adenoviral vectors, Current Protocols in Neuroscience, Unit 4 23, Chapter 4; Akli et al. (1993)Transfer of a foreign gene into the brain using adenovirus vectors. Nature genetics, 3(3): 224-228.

Another aspect provides a vector as described herein, typically a viral vector as described herein.

Viral vectors are typically packaged into viral particles using methods known in the art. The viral particles may then be used to transfer cell lines, including neural cell lines, or neural tissue, either in vitro or in vivo. Thus, another aspect provides a viral particle comprising a vector described herein.

A further aspect provides an agent comprising a tau gene editing system, or part therefor, or a nucleic acid encoding a tau gene editing system, or part thereof, as described herein. In one embodiment, the tau gene editing system or part thereof introduces a mutation into the wild-type tau gene, typically the endogenous tau gene, to cause expression of a phosphomimetic tau. Typically, the phosphomimetic tau is T205E or T205D. In one embodiment, the tau gene editing system comprises CRISPR/Cas, typically CRISPR/Cas9, which targets the wild-type tau gene, in combination with a donor nucleic acid which introduces into the wild-type tau gene, typically the endogenous wild-type tau gene, a substitution of threonine for glutamic acid or aspartic acid in the sequence SSPGSPGTPGSRSR of tau, typically the threonine at position 205 of wild-type human tau.

Typically, the tau gene editing system, or part thereof, comprises a guide RNA (gRNA), or a nucleic acid encoding a guide RNA, that is complementary to a portion of the coding region of the tau gene sequence, typically at or near sequence encoding T205 of tau. Typically, the gRNA is a single guide RNA or a pair of guide RNAs. Examples of suitable pairs of guide RNA sequences for targeting mouse and human tau comprise the sequences shown below:

Guide RNA 1:
Mouse:
(SEQ ID NO: 69)
CGAGCGACTGCCAGGCGTTC
Human:
(SEQ ID NO: 70)
GGAGCGGCTGCCGGGAGTGC
Guide RNA 2:
Mouse:
(SEQ ID NO: 71)
CCCGGCTCTCCCGGAACGCC
Human:
(SEQ ID NO: 72)
CCCGGCTCCCCAGGCACTCC

Typically, the tau gene editing system comprises a CRISPR/Cas9 complex in combination with a donor nucleic acid.

Typically, the guide RNA further comprises sequence which binds an endonuclease, such as for example the Cas protein, typically Cas9 protein. Typically, the guide RNA comprises a protospacer-adjacent motif (PAM) sequence and a fusion of the bacterial crRNA and tracrRNA for binding Cas protein. The guide RNA thus provides both targeting specificity and scaffolding/binding ability for the Cas endonuclease. In this regard, the guide RNA sequence directs the endonuclease, such as Cas, to the target site, where the nuclease creates a double strand break in the target DNA. In order to introduce mutations using the tau gene editing system, the gRNA and Cas protein, or nucleic acid encoding Cas protein, are introduced into a cell together with a donor sequence comprising a mutant tau sequence for incorporating into the endogenous wild-type tau gene. The donor nucleic incorporates the mutant tau sequence into the tau allele, typically by homology directed repair. An Example of donor sequences for mutating the mouse and human tau gene (MAPT gene) is as follows:

Mouse:
(SEQ ID NO: 73)
CCAGGTGAACCACCAAAATCCGGAGAACGAAGCGGCTACAGCAGCCCCG
GCTCTCCCGGAGAGCCTGGCAGTCGCTCGCGCACCCCATCCCTACCAAC
ACCGCCCACCCGGGAGCCCAAG
Human:
(SEQ ID NO: 6)
tCtGGTGAACCtCCAAAATCaGGgGAtCGcAGCGGCTACAGCAGCCCCG
GCTCcCCaGGcGAGCCcGGCAGcCGCTCcCGCACCCCgTCCCTtCCAAC
cCCaCCCACCCGGGAGCCCAAG

In one embodiment, the donor sequence for mutating the human tau gene comprises a nucleotide sequence encoding an amino acid sequence selected from the group consisting of SPGSPGXPGSRSR (SEQ ID NO: 74), SSPGSPGXPGSRSRT (SEQ ID NO: 75), SSPGSPGXPGSRSRT (SEQ ID NO: 76), YSSPGSPGXPGSRSRTP (SEQ ID NO: 77), GYSSPGSPGXPGSRSRTPS (SEQ ID NO: 78), SGYSSPGSPGXPGSRSRTPSL (SEQ ID NO: 79),

(SEQ ID NO: 80)
RSGYSSPGSPGXPGSRSRTPSLP,
(SEQ ID NO: 81)
DRSGYSSPGSPGXPGSRSRTPSLPT,
(SEQ ID NO: 82)
GDRSGYSSPGSPGXPGSRSRTPSLPTP,
(SEQ ID NO: 83)
SGDRSGYSSPGSPGXPGSRSRTPSLPTPP,
(SEQ ID NO: 84)
KSGDRSGYSSPGSPGXPGSRSRTPSLPTPPT,
(SEQ ID NO: 85)
PKSGDRSGYSSPGSPGXPGSRSRTPSLPTPPTR,
(SEQ ID NO 86)
PPKSGDRSGYSSPGSPGXPGSRSRTPSLPTPPTRE,
(SEQ ID NO: 87)
EPPKSGDRSGYSSPGSPGXPGSRSRTPSLPTPPTREP,
(SEQ ID NO: 88)
GEPPKSGDRSGYSSPGSPGXPGSRSRTPSLPTPPTREPK,
and
(SEQ ID NO: 89)
SGEPPKSGDRSGYSSPGSPGXPGSRSRTPSLPTPPTREPK,

wherein X is E or D.

In one embodiment, the donor sequence for mutating the human tau gene comprises a nucleotide sequence that is at least 60%, more typically at least 65%, at least 70%, at least 75%, at least 80%, at least 85%, at least 90%, at least 95%, or at least 99%, identical to SEQ ID NO: 6.

In one embodiment, the donor sequence for mutating the human tau gene comprises a nucleotide sequence that is at least 60%, more typically at least 65%, at least 70%, at least 75%, at least 80%, at least 85%, at least 90%, at least 95%, or at least 99%, identical to SEQ ID NO: 6, and encodes the amino acid sequence SGEPPKSGDRSGYSSPGSPGXPGSRSRTPSLPTPPTREPK, wherein X is E or D.

Methods for genome editing using CRISPR/Cas9 are known in the art and are described in, for example, U.S. Pat. No. 10,240,145; and US 201180127783;

In various embodiments:

(i) gRNA is administered with RNA encoding Cas protein, and ds or ss donor DNA;

(ii) gRNA is administered with Cas protein, and ds or ss donor DNA;

(iii) DNA encoding gRNA is administered with DNA encoding Cas protein, and ds or ss donor DNA;

(iv) DNA encoding gRNA is administered with Cas protein, and ds or ss donor DNA;

(v) DNA encoding gRNA is administered with RNA encoding Cas protein, and ds or ss donor DNA.

It will be appreciated that DNA encoding gRNA or DNA encoding Cas protein comprises the gRNA sequence or Cas coding sequence operably linked to suitable regulatory sequences as described herein.

It will also be appreciated that each of the components of the tau editing system (e.g., gRNA, endonuclease and donor sequence) can be administered together, or separately.

In some embodiments, the tau gene editing system is introduced into neurons of the subject via a vector. For example, the tau gene editing system may be introduced into neurons of the subject in an AAV vector system.

The agent described herein may be formulated as a pharmaceutical composition. Accordingly, in another aspect, there is provided a pharmaceutical composition comprising the agent described herein. The composition comprises the agent in a pharmaceutically acceptable carrier. Methods for the formulation of agents with pharmaceutical carriers are known in the art and are described in, for example, Remington's Pharmaceutical Science, (17^{th} ed. Mack Publishing Company, Easton, Pa. 1985); Goodman & Gillman's: The Pharmacological Basis of Therapeutics (11^{th} Edition, McGraw-Hill Professional, 2005).

Acceptable carriers, diluents and adjuvants are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics or polyethylene glycol (PEG).

Administration of the agent to subject may be by intracranial, intravenous, intraperitoneal, subcutaneous, intramuscular, intranasal or intrathecal injection. Compositions suitable for intracranial, intravenous, intraperitoneal, subcutaneous, intramuscular, intranasal or intrathecal use include sterile aqueous solutions or dispersions and sterile powders for the extemporaneous preparation of sterile injectable solutions or dispersions. The pharmaceutically acceptable carrier can be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride.

In embodiments in which the agent is packaged in a viral particle, the pharmaceutical compositions may comprise viral particles in any concentration that allows the agent to be effective. In such embodiments, the pharmaceutical compositions may comprise the virus particle in an amount of from 0.1% to 99.9% by weight. Pharmaceutically acceptable carriers include water, buffered water, saline solutions such as, for example, normal saline or balanced saline solutions such as Hank's or Earle's balanced solutions), glycine, hyaluronic acid etc.

Titers of viral particles to be administered will vary depending on, for example, the particular vector to be used, the mode of administration, extent of the condition, the individual, and may be determined by methods standard in the art.

The agent described herein may be formulated for introduction into neuronal cells by non-viral methods such as microinjection, electroporation, microparticle bombardment, liposome uptake, nanoparticle-based delivery etc.

In one embodiment, the agents described herein may be formulated in one or more liposomes, lipoplexes, or lipid nanoparticles. In one embodiment, the agents described herein are formulated in liposomes. Liposomes are unilamellar or multilamellar vesicles which have a membrane formed from a lipophilic material and an aqueous interior. The aqueous portion contains the composition to be delivered. Liposome design may include, for example, opsonins or ligands in order to improve the attachment of liposomes to tissue or to activate events such as, for example, endocytosis.

The formation of liposomes may depend on the physicochemical characteristics such as the agent and the liposomal ingredients, the nature of the medium in which the lipid vesicles are dispersed, the effective concentration of the agent, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

Methods for the production of liposomes and lipid nanoparticles for delivery of agents are known in the art, and described in, for example, U.S. Pat. No. 5,264,221.

The term “administering” should be understood to mean providing a compound or agent to a subject in need of treatment.

The term “ effective amount” refers to the amount of an agent that will elicit the biological response of a system, tissue, or subject that is being sought.

It will be understood that the specific dose level and frequency of dosage for any particular subject may be varied and will depend upon a variety of factors including, for example, the activity of the specific compound or agent employed, the metabolic stability and length of action of that compound or agent, the age, body weight, general health, sex, diet, mode and time of administration, drug combination, the severity of the particular condition, and the subject undergoing therapy.

Also provided is a kit, comprising a container comprising the agent. The container may be simply a bottle comprising the agent in parenteral dosage form, each dosage form comprising a unit dose of the agent. The kit will further comprise printed instructions. The article of manufacture will comprise a label or the like, indicating treatment of a subject according to the present method. In one form, the article of manufacture may be a container comprising the agent in a form for parenteral dosage. For example, the agent may be in the form of an injectable solution in a disposable container.

As used herein, “treating” means affecting a subject, tissue or cell to obtain a desired pharmacological and/or physiological effect and includes inhibiting the condition, i.e. arresting its development; or relieving or ameliorating the effects of the condition i.e. cause reversal or regression of the effects of the condition.

As used herein, “preventing” means preventing a condition from occurring in a cell or subject that may be at risk of having the condition, but does not necessarily mean that condition will not eventually develop, or that a subject will not eventually develop a condition. Preventing includes delaying the onset of a condition in a cell or subject.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

All publications mentioned in this specification are herein incorporated by reference. It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

The present application claims priority from Australian provisional application no. 2019903530, the entirety of which is incorporated herein by reference.

In order to exemplify the nature of the present invention such that it may be more clearly understood, the following non-limiting examples are provided.

### EXAMPLES

### Materials and Methods

### Mice

APP23 mice expressing human K670N/M671L mutant APP in neurons (Sturchler-Pierrat et al., 1997), ALZ17 mice expressing human non-mutant tau in neurons (Probst et al., 2000), tau^{-/-} (Tucker et al., 2001), p38γ^{-/-} {Perdiguero, 2007 #164} mice, transgenic mice expressing constitutively active p38γ^{CA} in neurons (Ittner et al., 2016) and transgenic Tau58 mice expressing P301S mutant human tau in the brain (van Eersel et al., 2015) were previously described. All lines were maintained on a C57B1/6 background. Animal experiments were approved by the Animal Ethics Committee of the University of New South Wales. Mice were genotyped by polymerase chain reaction using isopropanol-precipitated DNA from tail biopsies as template. Oligonucleotide primers for genotyping targeted alleles and transgenes by polymerase chain reaction (PCR) are described in Table 1.

### Genome Editing

The murine Mapt gene was targeted using CRISPR/Cas9 as described previously (Delerue and Ittner, 2017; Yang et al., 2014). Briefly, two guides targeting codon T194 of the endogenous murine Mapt gene (homologous to the human codon T205) were designed using the computational tool {Ran, 2013 #417} (http://crispr.mit.edu). These single-guide RNAs (sgRNAs) were generated using a non-cloning method whereby a T7-conjugated forward primer was used to generate a linear template by PCR. The sgRNA scaffold of the pX330 (Addgene #42230, gift from Dr Feng Zhang) was used as a template. The resulting linear DNA was in-vitro transcribed into sgRNAs using a T7 Quick High Yield RNA synthesis kit, following the manufacturer's instructions (NEB #E2050S). sgRNAs were purified using NucAway Spin columns (ThermoFisher #AM10070). Pronuclear injections of Cas9 protein (NEB #M0646T), sgRNAs and donor single-stranded oligos (ssOligos) into C57B1/6 zygotes resulted in live pups. Initial identification of founders was performed by sequencing of individual alleles for each pup by cloning. Briefly, sequence of Mapt exon 9 (ENSEMBL ENSMUST00000106989.2) was amplified by PCR from mouse DNA and cloned into pBluescript (Stratagene) using HiFi Assembly (New England Biolabs). At least 5 clones per pup were sequenced to identify potential founders with correct codon exchange at Threonine-194. Founders with verified codon exchange were crossed with C57B1/6 mice to establish the tauT205A and tauT205E lines, respectively. TauT205A and tauT205E mice were genotyped using isopropanol-precipitated DNA from tail biopsies as template for tetra-primer ARMS-PCR{Ye, 2001 #395}. Oligonucleotide primers for tetra-primer ARMS PCR were designed using Primer1 (http://primer1.soton.ac.uk/primer1.html). Sequences for guide RNAs, homologous repair templates for codon exchange at T205 in Mapt and oligonucleotide primers for tetra-primer ARMS PCR are listed in Table 1.

TABLE 1
Oligonucleotide primers used for genotyping of mouse strains.

| | Forward primer | Reverse primer |
|---|---|---|
| Strain | (5′-3′) | (5′-3′) |
| APP23 | GTTCTGCTGCATCTTGGAC | GAATTCCGACATGACTCAGG |
| | A (SEQ ID NO: 90) | (SEQ ID NO: 91) |
| Alz17 | GGGTGTCTCCAATGCCTGC | AAGTCACCCAGCAGGGAGGT |
| | TTCTTCAG (SEQ ID NO: 92) | GCTCAG (SEQ ID NO: 93) |
| p38γ | TGGGCTGCGAAGGTAGAGG | GTGTCACGTGCTCAGGGCCT |
| | TG (SEQ ID NO: 94) | G (SEQ ID NO: 95) |
| tau^{WT} | CTCAGCATCCCACCTGTAA | CCAGTTGTGTATGTCCACCC |
| | C (SEQ ID NO: 96) | (SEQ ID NO: 97) |
| tau^{KO} | AAGTTCATCTGCACCACCG | TGCTCAGGTAGTGGTTGTCG |
| | (SEQ ID NO: 98) | (SEQ ID NO: 99) |
| Thy1.2- | AAGTCACCCAGCAGGGAGG | TCGTATGGGTACATGGCCAA |
| p38γCA | TG (SEQ ID NO: 100) | AG (SEQ ID NO: 101) |
| tauT205A/ | CAGCCCCGGCTCTCCCGTA | CGCGAGCGACTGCCAGGAGT |
| E_4ARMS_I | G (SEQ ID NO: 102) | (SEQ ID NO: 103) |
| tauT205A/ | TGTATCAAAGTGACAGAGC | TGGTGCTTCAGGTTCTCAGT |
| E_4ARMS_O | AGGAGTGATGC (SEQ ID NO: | AGAGCCAA (SEQ ID NO: 105) |
| | 104) | |

### Memory Testing

Spatial learning/memory was tested in the Morris Water maze (MWM) paradigm {Vorhees, 2006 #35;Ittner, 2016 #217;Tan, 2018 #390}. Briefly, a custom-built water tank for mouse MWM (122 cm diameter, 50 cm height) with white non-reflective interior surface in a room with low-light indirect lighting was filled with water (19-22° C.) containing diluted non-irritant white dye. Four different distal cues were placed surrounding the tank at perpendicular positions of the 4 quadrants. In the target quadrant (Q1), a platform (10 cm²) was submerged 1 cm below the water surface. Videos were recorded on CCD camera and analyzed using AnyMaze Software. For spatial acquisition, four trials of each 60 seconds were performed per session. The starting position was randomized along the outer edge of the start quadrant for all trials. To test reference memory, probe trials without platform were performed for a trial duration of 60 seconds, and recordings were analyzed for time spent within each quadrant. For visually-cued control acquisition (to exclude vision impairments), a marker was affixed on top of the platform and four trials (60 s) per session were performed. All mice were age and gender-matched and tested at 4 months of age. Mice that displayed continuous floating behavior were excluded. Genotypes were blinded to staff recording trials and analyzing video tracks. Tracking of swim paths was done using the AnyMaze software (Stolting). Average swimming speed was determined to exclude motor impairments.

### Behavioral Testing

Mice were tested for 10 minutes on the elevated plus maze to assess disinhibition and anxiety using a standard protocol previously published (Ke et al., 2015).

### Seizures

Seizures were induced with pentylenetetrazole (PTZ, Sigma-Aldrich) as previously described (Ittner et al., 2010). Briefly, PTZ was injected i.p. at 30 or 50 mg/kg body weight. Seizures were graded as: 0, no seizures; 1, immobility; 2, tail extension; 3, forelimb clonus; 4, generalized clonus; 5, bouncing seizures; 6, full extension; 7, status epilepticus.

### Plasmid Constructs

Oligonucleotide primers for PCR-based generation of plasmid constructs are listed in Table 2. Constructs for generation of AAV particles were based on pAAV-hsynl-eGFP-WPRE (Addgene #58867). eGFP coding sequence was replaced with the p38γ^{CA} coding sequence (p38γ Asp179A1a), tauWT (human tau40 441 amino acids) or tauT205A using HiFi Assembly (New England Biolabs) as described in (Ittner et al., 2016). A tau construct with the codon exchange T205A was described previously (Ittner et al., 2016). All plasmids were amplified in E. coli DH5α or XL-1blue. AAV vectors were propagated in E. coli Stb13 to avoid recombination events. All constructs were verified by sequencing.

TABLE 2
Oligonucleotide primers for molecular cloning.

| DNA | | |
|---|---|---|
| construct | Forward primer (5′-3′) | Reverse primer (5′-3′) |
| pAAV- | CCTGAAAGAAgatCCCCTGCA | CCAGCGTCCGTGTCACCC |
| hSyn1-p38γCA | GACCCCC (SEQ ID NO: 106) | (SEQ ID NO: 107) |
| pAAV- | CAAGCCCAGCAATGCCTACC | CGAGGTTGTGATGTCTGG |
| hSyn1-tau | TGAGTGACGTGAGCAAGGGCGAGG | GGGAGCATAGCTCTTGTACAGC |
| | AGG (SEQ ID NO: 108) | TCGTCCATGCC (SEQ ID NO: |
| | | 109) |

### Adeno-Associated Virus Production and Application

Packaging of AAV vectors was performed as described {Harasta, 2015 #80}. In brief, for packaging of AAV particles, 293T cells were seeded in complete DMEM (Sigma) with 10% FBS at 70-80% confluence. Culture medium was changed to IMDM (Sigma) with 5% FBS 3 hours prior to transfection. Cells were transfected with viral genome-containing plasmid, pFdelta6 as helper plasmid and AAV-PHP.B plasmid containing rep and cap sequences using polyethyleneimine-Max (PEI-Max, Polysciences) as a transfection reagent. Cells and supernatant were harvested 72 hours post transfection. Supernatant was clarified by adding 40% PEG8000/2.5M NaCl to a final concentration of 8%

PEG8000/0.5M NaCl and incubated at 4° C. for at least 2 hours. Clarified supernatant was centrifuged at 2000 g for 30 mins. Combined precipitate from clarified supernatant and cell pellet was treated with sodium deoxycholate (0.5% final concentration) and benzonase (˜500 U) at 37° C. for 40 mins. After addition of NaCl, incubation at 56° C. for 40 mins and freeze-thaw, the solution was centrifuged 30 min at 5000 g at 4° C. Supernatants were purified using iodixanol gradient by ultracentrifugation (475,900 g for 2 h at 18° C.). AAV particles were concentrated and exchanged into PBS in an Amicon 100 kDa 15 mL concentrator at 5000 g at 4° C. After titering with qPCR, aliquots were stored at −80° C. Titres were determined by quantitative polymerase chain reaction (qPCR). AAV titres were (in viral genomes per ml): AAV-PHP.B-syn1-eGFP (1.46×10¹⁴), AAV-PHP.B-syn1-p38γ^{CA} (1.37×10¹⁴), AAV-PHP.B-syn1-tauWT (8.42×10¹³), AAV-PHP.B-syn1-tauT205A (2.80×10¹³). Application either at postnatal day 0 (P0) using undiluted AAV or intravenous (i.v.) with dilution in sterile saline (0.9% NaCl). For PO injections, 1 μl (1×10⁹ viral particles) of AAV particles was injected at 3 sites each bilaterally into the brains of cryo-anaesthetized neonatal mice as described (Ittner et al., 2016). For systemic delivery, 100 μl of AAV particle solution (at either 10¹¹ or 10¹³ virion particles/ml) were injected into the tail vein of mice.

### Mouse Brain Lysates and Immunoblotting

Mouse cortical tissue was extracted after transcardial perfusion with phosphate-buffered saline (PBS pH7.4). Cortical tissues were homogenized in RIPA buffer (20 mM Tris pH8.0, 150 mM NaCl, 1 mM EDTA, 1 mM Na₃VO₄, 1 mM NaF, 1 mM glycerophosphate, 2.5 mM Na₂H₂P₂O₇, 1 mM PMSF, protease inhibitors (Complete, Roche), 1% NP-40 substitute (Sigma-Aldrich), 0.1% SDS, 0.5% sodium deoxycholate) on ice using a dounce homogenizer (Heidolph). Lysates were cleared by centrifugation (16,000×g/10 min/4° C.). Protein concentration was determined (DC Protein Assay, BioRad). Western blotting was performed as previously described (Ittner et al., 2012). Bands were visualized by chemiluminescence on X-ray films or digital imaging system (ChemiDoc MP, Biorad). Densitometric quantification of Western blot results was performed using ImageJ 2.0.0-rc-49/1.51d (NIH). Antibodies used in this study were: anti-PSD95 (Millipore), anti-Aβ (6E10), anti-tau (DAKO), anti-tau (tau-1, Millipore), anti-tau (Tau13, Abcam), anti-phospho-Threonine205 tau (Abcam), anti-phospho-Serine214 tau (Millipore), anti-glyceraldehyde dehydrogenase (anti-GAPDH, Millipore), anti-p38γ (R&D), anti-HA7 (Sigma), anti-HA (Cell Signaling Technologies), anti-SNAP25 (Millipore), anti-neurofilament (NF200, Abcam).

### Immunoprecipitation

Immunoprecipitation was performed from tissue lysates as previously described (Ittner et al., 2010). Briefly, cortical or hippocampal tissues were homogenized in RIPA buffer (20 mM Tris pH8.0, 150 mM NaCl, 1 mM EDTA, 1 mM Na₃VO₄, 1 mM NaF, 1 mM glycerophosphate, 2.5 mM Na₂H₂P₂O₇, 1 mM PMSF, protease inhibitors (Complete, Roche), 1% NP-40 substitute (Sigma-Aldrich), 0.1% SDS, 0.5% sodium deoxycholate) on ice. Lysates were cleared by centrifugation (16,000×g/10 min/4° C.). Protein concentration was determined (DC Protein Assay, BioRad) and 200 μg of lysate incubated with antibody (1:400) for 3 h on a rotator at 4° C. Equilibrated and blocked protein G-beads (New England Biolabs) were incubated with lysates for 45 min on a rotator at 4° C. Beads were then washed 3 times and incubated in sample buffer for 5 min at 95° C. before SDS-PAGE. Quantitative densitometric analysis was performed using Image J 2.0.0-rc-49/1.51d (NIH).

### Histological Sections and Staining

Mice were transcardially perfused with phosphate-buffered saline followed by 4% paraformaldehyde (PFA) and post-fixing in 4% PFA overnight. Tissue was processed in an Excelsior tissue processor (Thermo) for paraffin embedding. Silver staining (Gallyas) to visualize tau aggregates and NFT-like structures in brain of tau transgenic mice was performed as previously described (Ittner et al., 2015). Brain sections from AAV-injected mice were stained with primary antibody to tau (Tau13; Abcam) or HA-tag (HA-7; Sigma-Aldrich) to visualize viral transgene expression. All tissue sections were imaged on a BX51 bright field/epifluorescence microscope (UPlanFL N lenses [∞/C.17/FN26.5]: 10×/0.3, 20×/0.5, 40×/0.75, 60×/1.25oil and 100×/1.3oil) equipped with a DP70 color camera (Olympus).

### Immunofluorescence

Immunofluorescence staining on histological tissue sections was done as previously described (Ittner et al., 2010). Briefly, tissue sections were deparaffinated, rehydrated, washed with phosphate buffered saline (PBS), treated with 0.02% NP-40 in PBS and blocked with blocking buffer (3% horse serum/1% bovine albumin in PBS). Primary antibodies diluted in blocking buffer were incubated over-night at 4° C. or for 1 hour at room temperature. After washing with PBS, secondary antibodies diluted in blocking buffer with or without addition of DAPI to visualize cell nuclei were incubated for 1 hour at room temperature. Cells were then washed and mounted using anti-fade mounting medium (Prolong Gold, Life Technologies). Secondary antibodies used were coupled to Alexa 488, 555, 568 or 647 dyes (Molecular Probes). Epifluorescence imaging was done on a BX51 bright field/epifluorescence microscope (UPlanFL N lenses [∞/0.17/FN26.5]: 10×/0.3, 20×/0.5, 40×/0.75, 60×/1.25oil and 100×/1.3oil) equipped with a DP70 color camera (Olympus) using CellSens software (Olympus). Silver impregnation of histological mouse brain sections (8 μm) was performed as previously described (Ittner et al., 2015). Amyloid plaque counts and size were determined on micrographs from cortical sections with immunofluorescence staining for amyloid-β (6E10) using Image J https://imagej.nih.gov/ij/). Counts of plaques were normalized to tissue surface area.

### Statistical Analysis

Statistical analysis including was performed using Graphpad Prism Version 6.0. Student's t-tests were performed for pairwise comparison, while multiple data groups were analyzed by ANOVA. Linear regression and correlation analysis was done by sum-of-squares minimization. Survival data were analyzed by log-rank Mantel-Cox testing. All values are presented as mean±standard error of the mean (SEM).

### Results

In one example, the inventors used the APP23 mouse models of

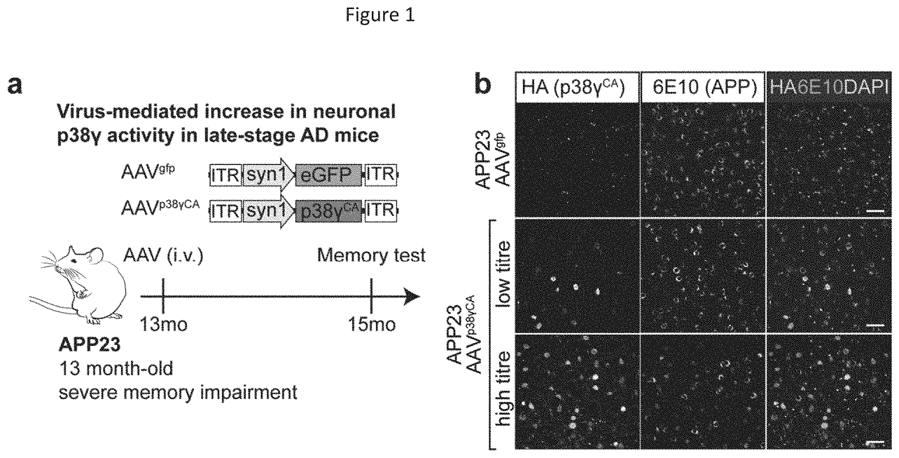

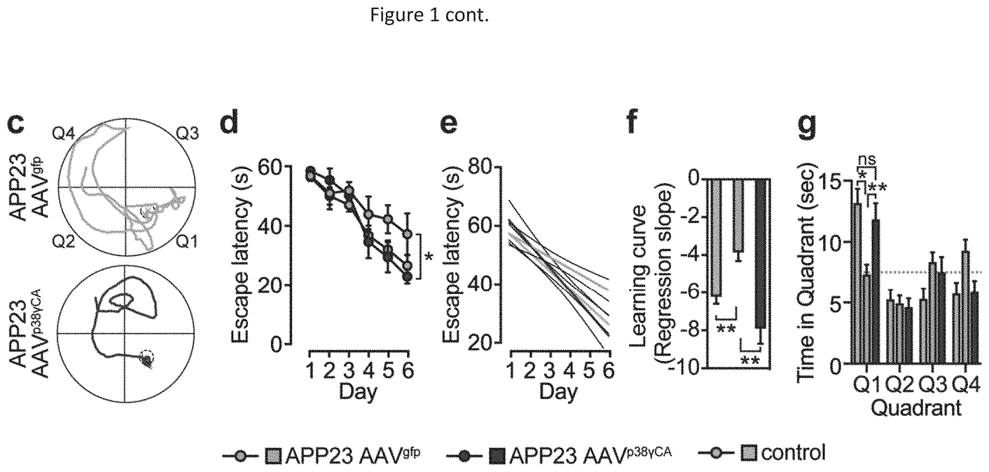

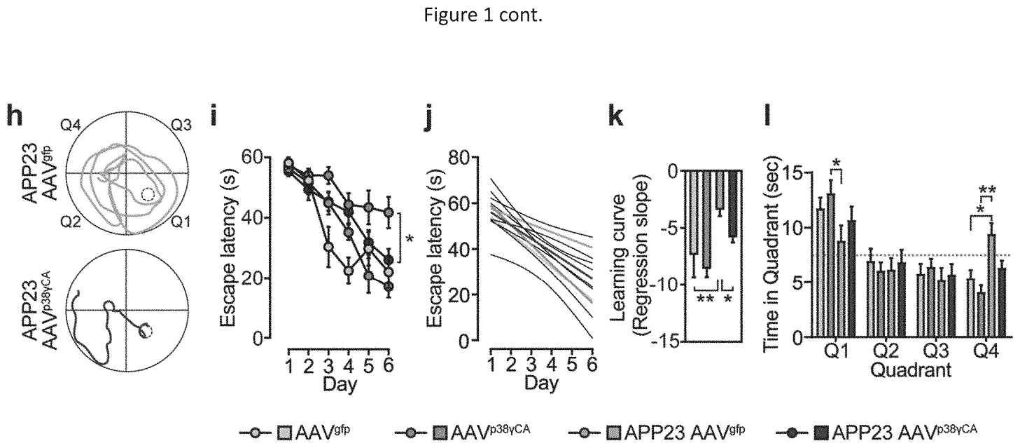

Alzheimer's disease with transgenic expression of human mutant amyloid-β precursor protein (APP) at an advanced aged of 13 months, where they have developed severe memory deficits, and treated them with different titres of AAV for neuronal expression of either constitutive active p38γ (p38γ^{CA}) or enhanced green fluorescence protein (eGFP) as a control (FIG. 1a). Memory performance was tested in the treated mice at 15 months of age (FIG. 1a). Expression of p38γ^{CA} in the brain of APP23 mice treated with either low (10¹⁰ AAV particles) or high (10¹² AAV particles) AAV titres were visualized by staining of the HA tag fused to p38γ^{CA} (FIG. 1b). Staining pattern of Aβ as visualized by the 6E10 antibody did not change with p38γ^{CA} expression. This confirmed successful expression of p38γ^{CA} in the brains of APP23 mice upon systemic AAV delivery.

Memory assessment of the mice in the Morris water maze (MWM) at 15 months of age showed significantly improved memory performance of APP23 mice treated with low tires of AAV-p38γ^{CA} as compared with persistent memory deficits in AAV-eGFP-treated APP23 littermates (FIG. 1c-g) . AAV-p38γ^{CA}-treated APP23 mice showed similar memory formation age-matched non-transgenic control mice. Taken together, these results demonstrate that the treatment of AD mice with low titres of AAV-p38γ^{CA} efficiently reverted their memory deficits at advanced ages.

Similarly, memory assessment of mice treated with higher titres of AAV-p38γ^{CA} or AAV-eGFP demonstrated efficient reversal of existing memory deficits in APP23 mice (FIG. 1h-l). Accordingly, treatment of aged APP23 mice with high titres of AAV-p38γ^{CA} showed comparable memory formation and consolidation as non-transgenic mice treated with AAV-p38γ^{CA} or AAV-eGFP, while APP23 mice treated with AAV-eGFP presented with continued poor memory performance during MWM testing. Taken together, these results demonstrate that the treatment of AD mice with high titres of AAV-p38γ^{CA} efficiently reverted their memory deficits at advanced ages.

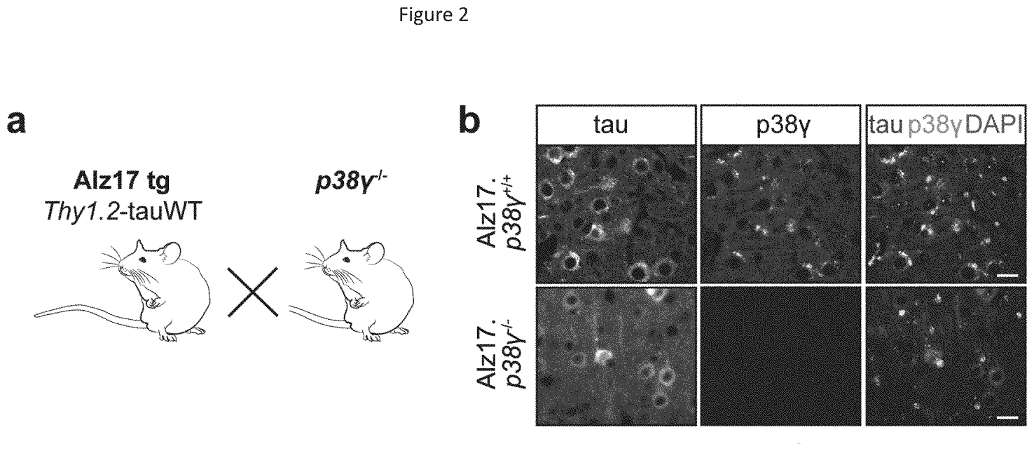

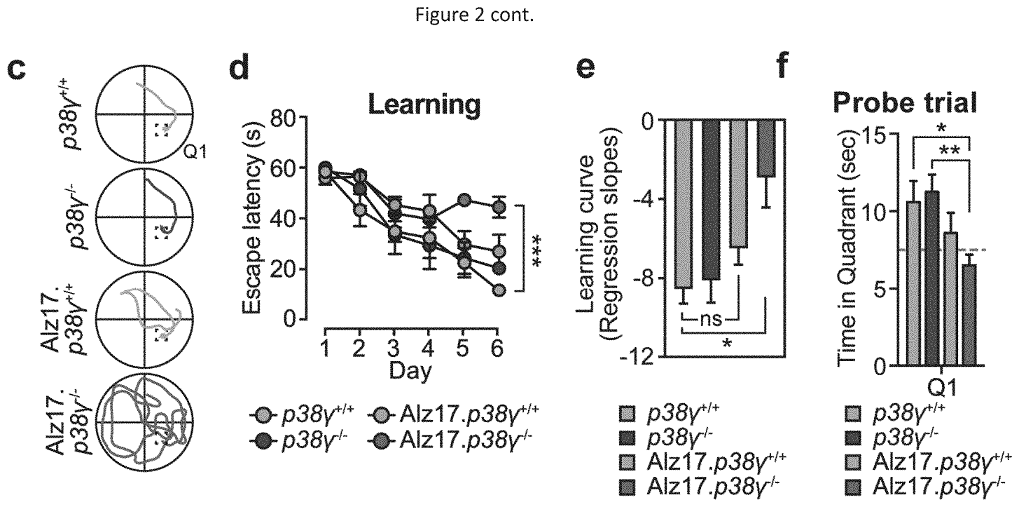

In another example, the inventors crossed human non-mutant tau transgenic Alz17 mice with p38γ-deficient p38γ^{-/-} mice (FIG. 2a). This resulted in the absence of p38γ in the brains of Alz17.p38γ^{-/-} mice, as visualized by immunostaining of endogenous p38γ and tau in the cortex of Alz17.p38γ^{-/-} as compared with Alz17.p38γ^{+/+} mice (FIG. 2b). This confirmed successful depletion of p38γ in the brains of Alz17.p38γ^{-/-} mice.

Memory assessment of Alz17.p38γ^{-/-} and Alz17.p38γ^{+/+} mice in the MWM confirmed absence of memory deficits in Alz17.p38γ ^{+/+} mice with comparable memory formation and consolidation to p38γ^{+/+} and p38γ^{-/-} littermates (FIG. 2c-f). In contrast, Alz17.p38γ^{-/-} mice presented with significantly delayed memory formation and markedly reduced memory consolidation as compared to Alz17.p38γ^{+/+} mice. This demonstrates that p38γ limits the toxic effects of increased levels of human hyperphosphorylated tau in the absence of Aβ in a mouse model of tauopathy.

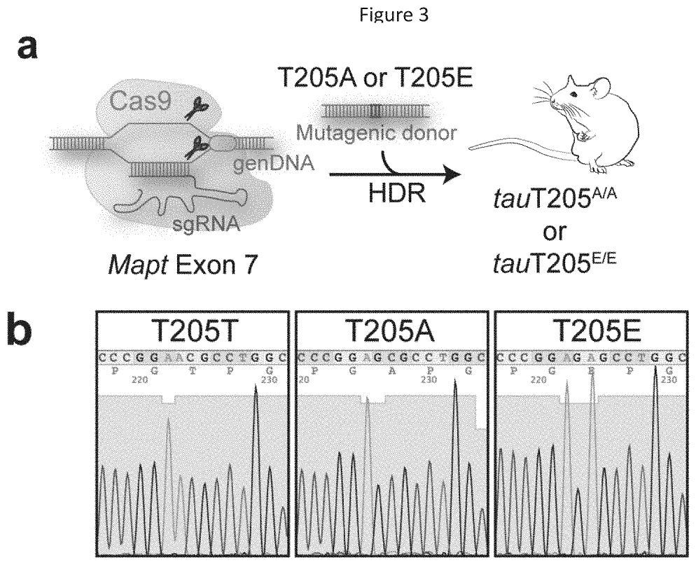

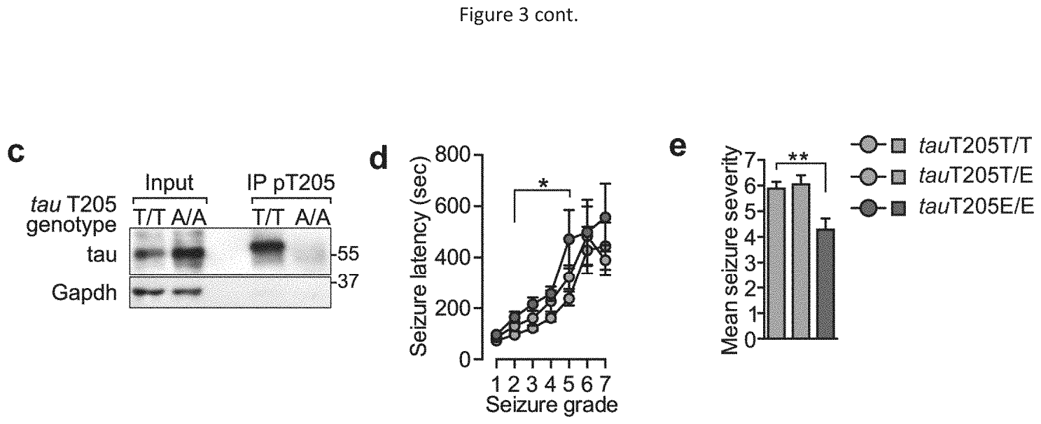

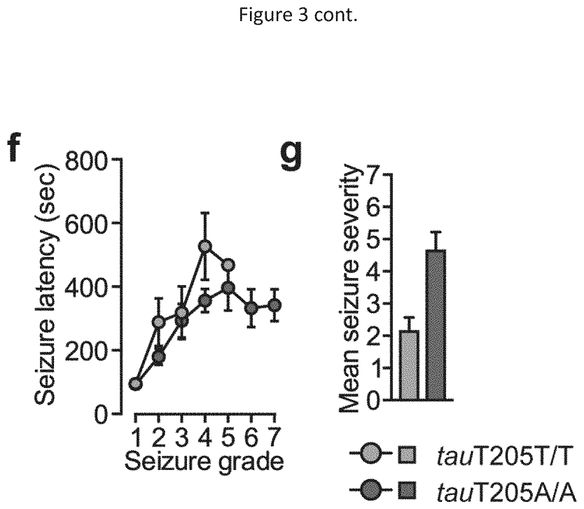

In another example, the inventors modified the endogenous murine Mapt gene that encodes tau using the CRSPR/Cas9 gene-editing technology to introduce either a T205E or T205A mutation in different mouse lines (FIG. 3a). The resulting lines were crossed to homozygosity to obtain T205E/E and T205A/A mice, respectively. Successful genome editing was confirmed by sequencing of genomic DNA obtained from both lines and compared to non-mutant T205T/T mice (FIG. 3b). Successful genome editing of T205A/A with resulting amino acid exchange in the translated murine protein was confirmed by immunoprecipitation with an antibody specific for tau phosphorylated at T205 (FIG. 3c). Taken together, the inventors have generated two novel mouse lines with mutant tau expression, T205E/E and T205A/A.

To determine the functional impact of the T205 variations in the endogenous tau protein, both T205E/E and T205A/A mice were subjected to the established excitotoxic test paradigm of induced seizures. T205E/E mice presented with increased latency to develop more severe seizures and reduced mean seizure severity as compared with rapidly developing and more severe seizures in heterozygous T205T/E and non-mutant T205T/T controls upon administration of 50 mg/kg pentylenetetrazole (FIG. 3d-e). Conversely, T205A/A mice showed more rapid development of more severe seizures and significantly increased mean seizure severity as compared to T205T/T littermates after injection of 30 mg/kg pentylenetetrazole (FIG. 3f-g). Taken together, this data demonstrates that the presence of the phosphorylation-mimicking T205E/E mutation reduced, while the phosphorylation-preventing T205A/A mutation augmented, susceptibility to induced excitotoxic seizures. Therefore, this data demonstrates that protective effects of T205 tau phosphorylation from epilepsy in vivo.

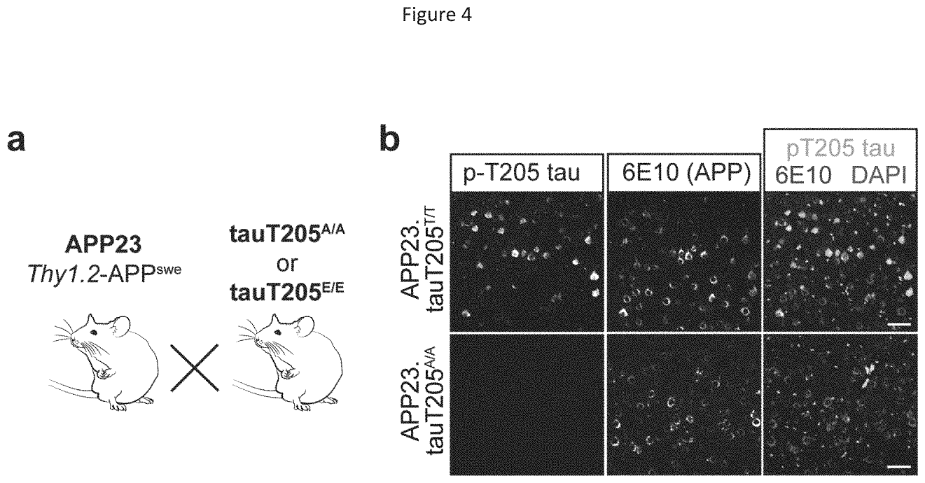

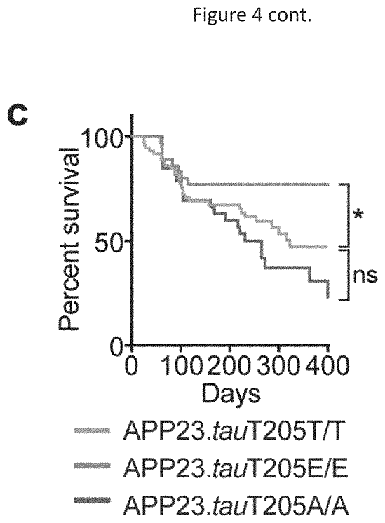

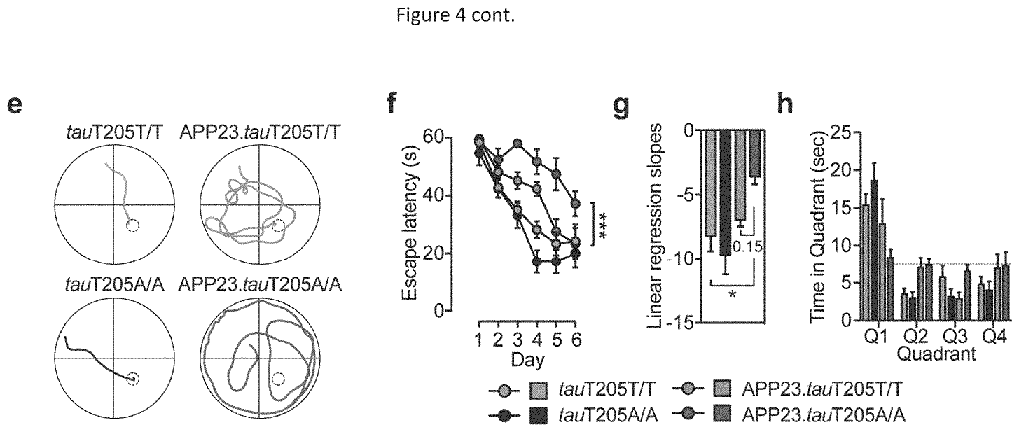

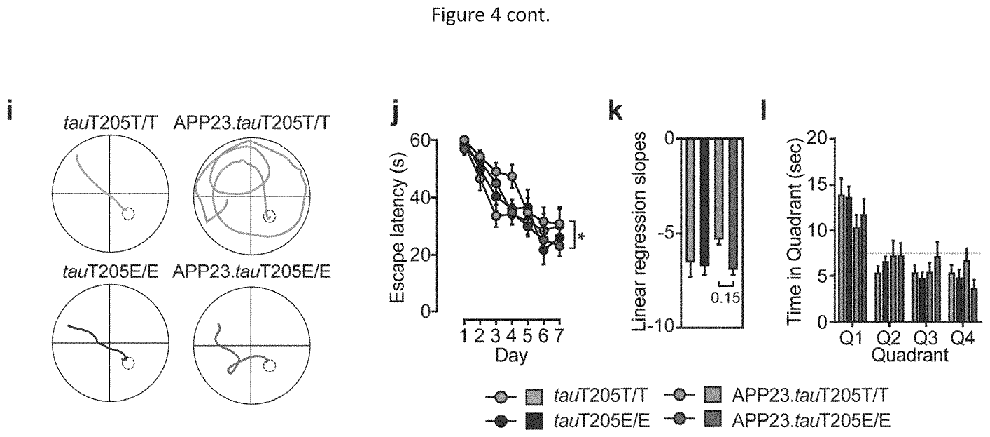

In another example, the inventors crossed T205E/E and T205A/A mice to APP23 mice to determine the role of T205 tau phosphorylation in memory deficits related to AD (FIG. 4a). Immunostaining of mouse brains with antibodies to tau phosphorylated at T205 (pT205) and Aβ (6E10) confirmed the absence of T205 tau phosphorylation in APP23.T205A/A mice (FIG. 4b). While APP23.T205E/E mice showed significantly improved survival as compared with APP23.T205T/T animals, while APP23.T205A/A showed a trend towards further accelerated mortality (FIG. 4c). This data demonstrates the critical role of T205 tau phosphorylation in regulating the survival of APP23 mice.

Memory testing in the MWM revealed further worsening of memory formation and consolidation in APP23.T205A/A mice compared to memory deficits in APP23.T205T/T mice (FIG. 4e-h). Conversely, APP23.T205E/E mice were prevented from developing memory deficits seen in APP23.T205T/T mice (FIG. 4i-l). Notably, T205A/A and T205E/E mice showed normal memory formation in the MWM. This data demonstrates that the phosphorylation of tau at T205 is limiting the memory deficits of APP23 mice, while it does not contribute to memory in the test paradigm in naive mice.

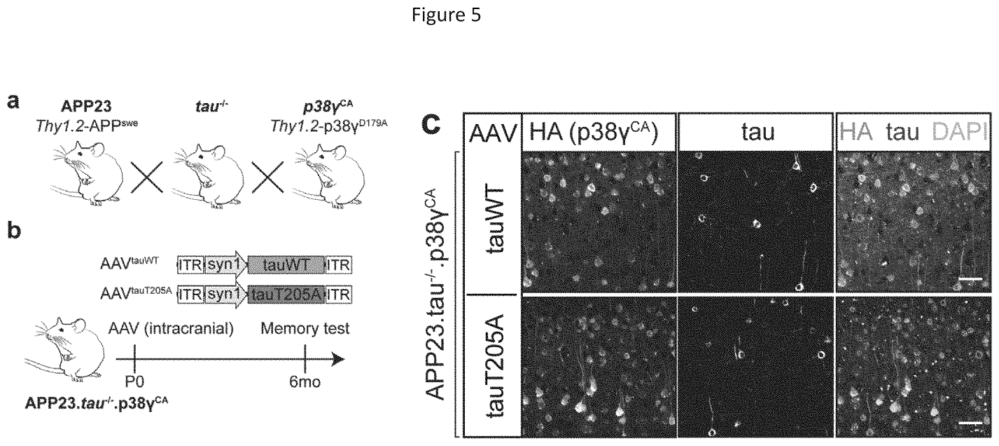

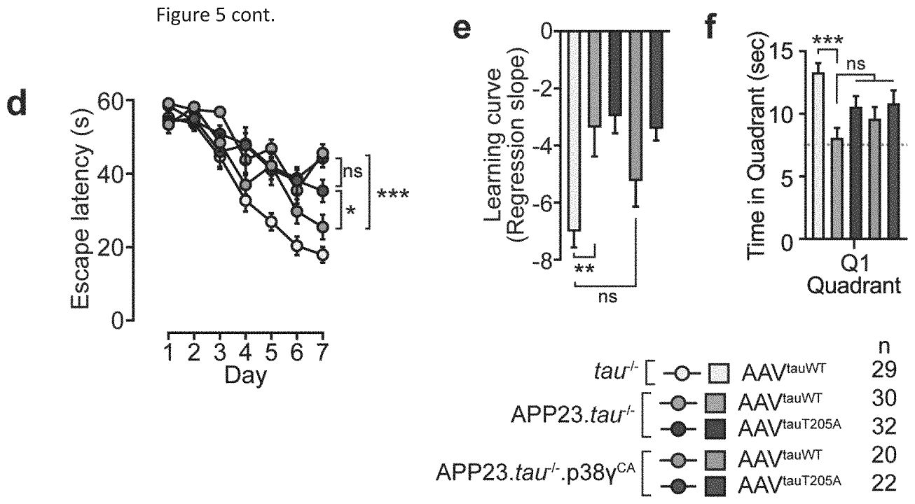

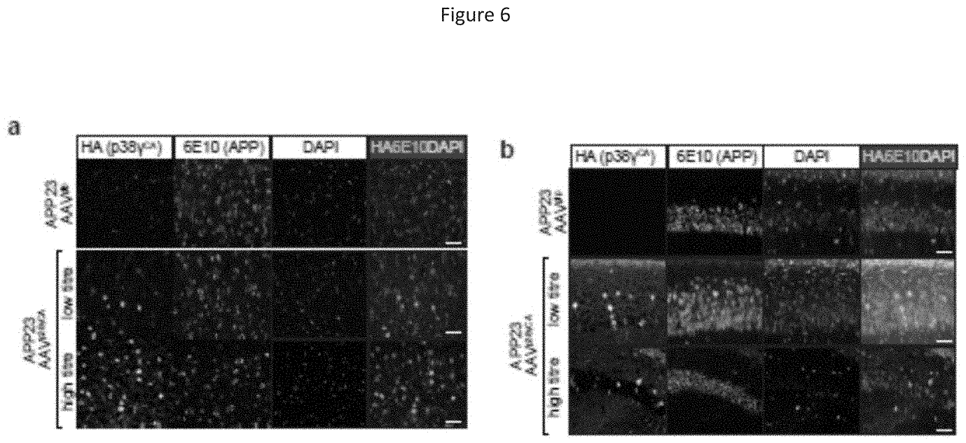

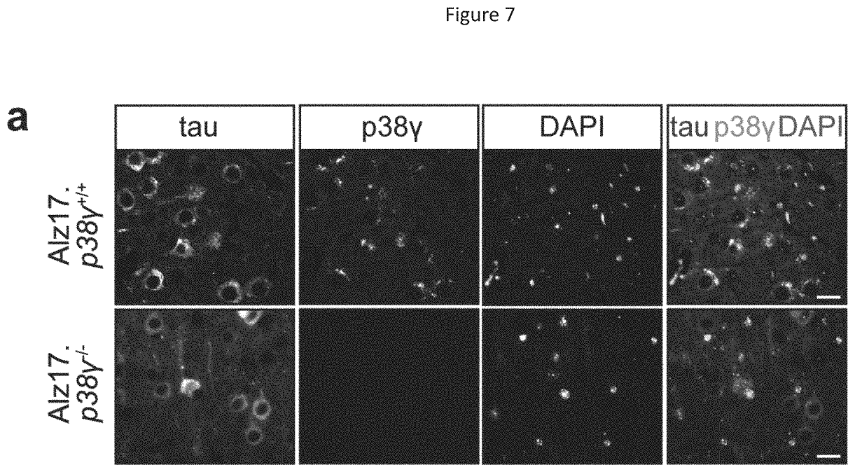

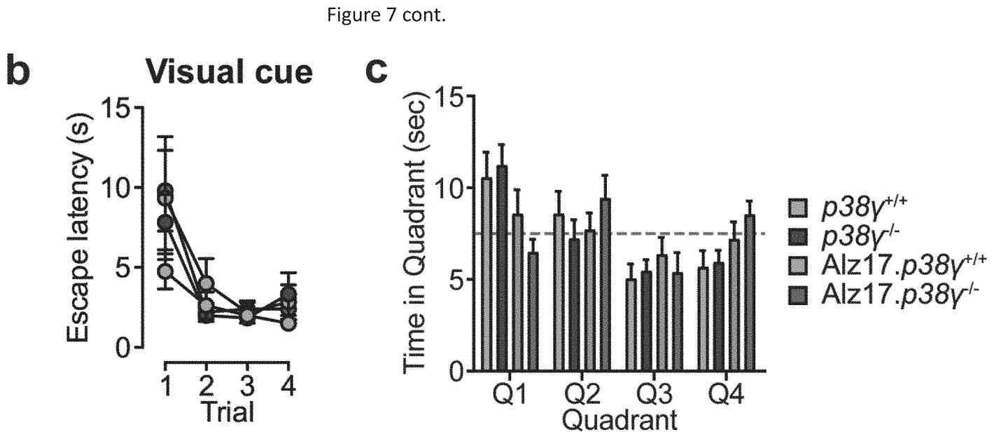

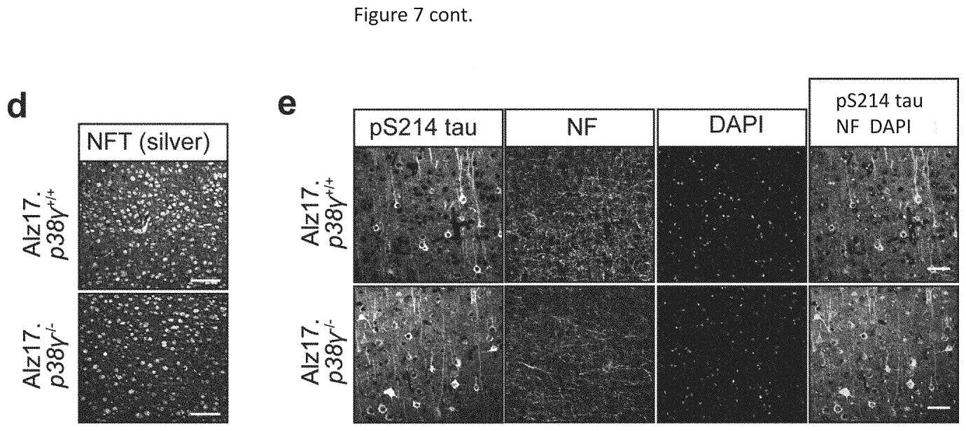

In another example, the inventors crossed APP23 mice on a tau-deficient background and further crossed the resulting APP23.tau^{-/-} mice with a line expressing transgenic p38γ^{CA} in neurons, producing APP23.tau^{-/-}.p38γ^{CA} mice (FIG. 5a). Tau expression in the brains of APP23.tau^{-/-}.p38γ^{CA} mice was then reconstituted by injection of AAV into the brains at birth, using tau variants of non-mutant tau (tauWT) or phosphorylation-preventing T205A tau (tauT205A) followed by memory testing at 6 months of age (FIG. 5b). Immunofluorescence staining with antibodies to the HA tag of p38γ^{CA} and to human tau of brains confirmed expression of p38γ^{CA} and successful reconstitution of tau in AAV-injected APP23.tau^{-/-}.p38γ^{CA} mice (FIG. 5c).

Memory testing of the AAV injected APP23.tau^{-/-}p38γ^{CA} mice revealed improved memory performance in APP23.tau^{-/-}.p38γ^{CA} mice injected with AAV-tauWT, while reconstitution with AAV-tauT205A failed to prevent memory deficits in APP23.tau^{-/-}.p38γ^{CA} mice (FIG. 5d-f). For comparison, both AAV-tauWT and AAV-tauT205A injection of APP23.tau^{-/-} mice (in the absence of p38γ) did not improve memory performance. TauWT-reconstituted tau^{-/-} mice were used as reference for normal memory task performance. Taken together, this data demonstrates that the phosphorylation of tau at T205 is required for the therapeutic efficacy of p38γ^{CA} in AD mice.

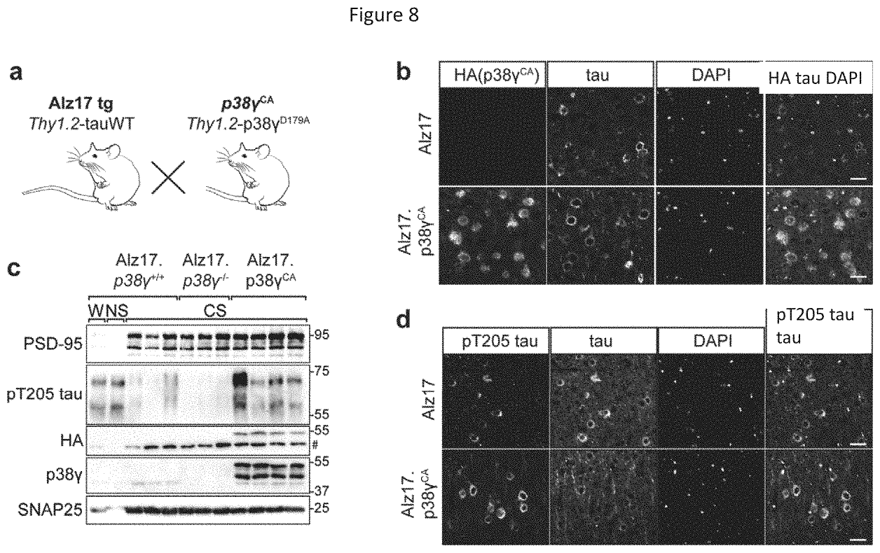

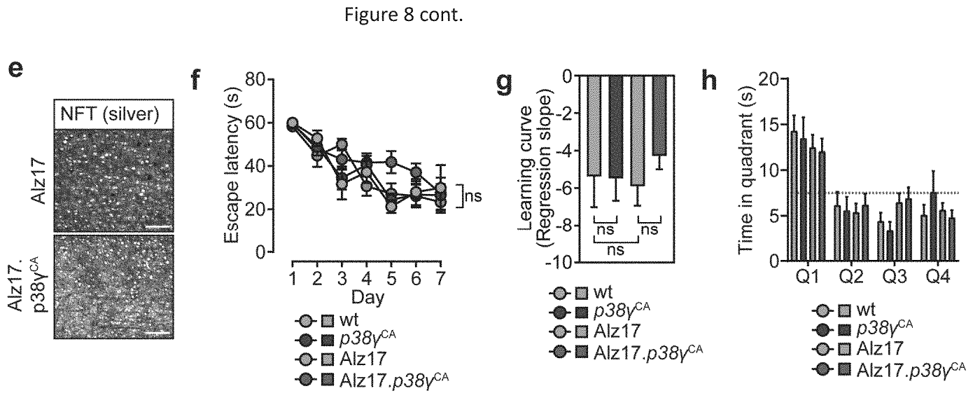

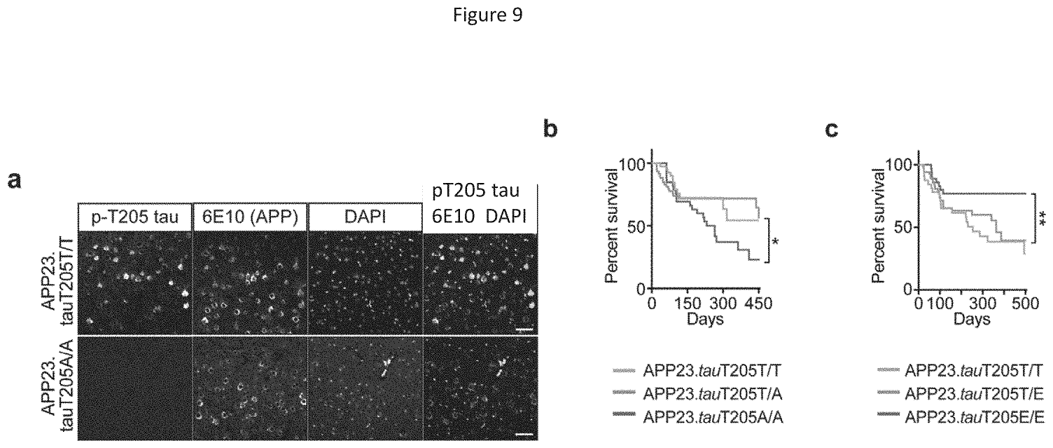

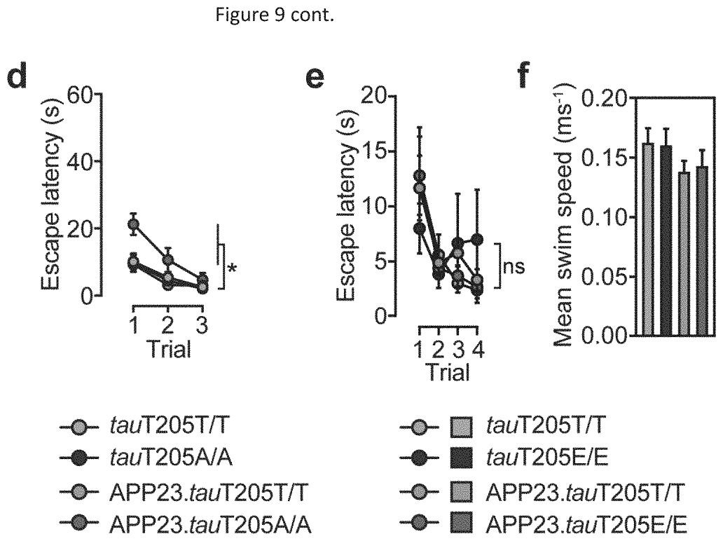

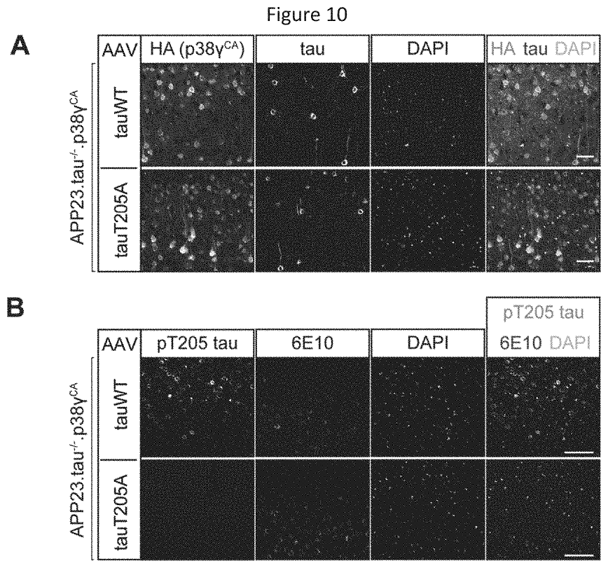

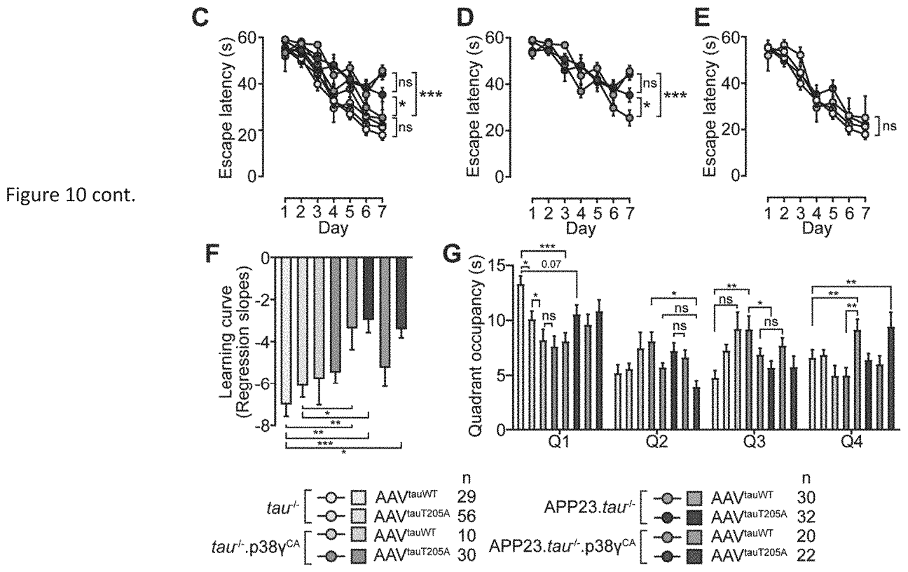

In another example, the inventors crossed human non-mutant tau transgenic Alz17 mice with transgenic mice that express p38γ^{CA} in neurons, resulting in Alz17.p38γ^{CA} mice (FIG. 8a). Immunofluorescence staining of brains with antibodies to the HA tag of p38γ^{CA} and human tau showed co-expression of tau and p38γ^{CA} in brain neurons (FIG. 8b). Western blotting of synaptosome preparations from mouse brains showed increased phosphorylation of tau at T205 in the post-synapse of Alz17.p38γ^{CA} mice as compared with Alz17.p38γ^{+/+} controls (FIG. 8c). In contrast, phosphorylation of tau at T205 was no longer detectable in Alz17.p38γ^{-/-} mice that also lacked p38γ in their post-synapses (FIG. 8c). Detection of the post-synaptic density protein 95 (PSD-95) and synaptosome-associated protein 25 (SNAP25) confirmed equal enrichment of synaptosomes during preparation. p38γ was present in synaptosomes of Alz17.p38γ^{+/+} and Alz17.p38γ^{CA} mice, while it was absent from synaptosome of Alz17.p38γ^{-/-} mice, consistent with their genotypes. Immunofluorescence staining of brains with antibodies to tau and tau phosphorylated at T205 revealed increased T205 tau phosphorylation in Alz17.p38γ^{CA} mice compared with Alz17 mice (FIG. 8d). However, silver staining of aged mouse brains showed no overt increase in neurofibrillary tangles (NFTs) in the brains of ALz17.p38γ^{CA} mice compared to Alz17 littermates (FIG. 8e). Memory testing of Alz17.p38γ^{CA} mice in the MWM showed no memory deficits and a performance that was indistinguishable from Alz17 or p38γ^{CA} or non-transgenic controls (FIG. 8f-h). Taken together, this data demonstrates that p38γ^{CA} expression in Alz17 mice increased T205 tau phosphorylation including at post-synapses but did not accelerate tau pathology or cause memory deficits.

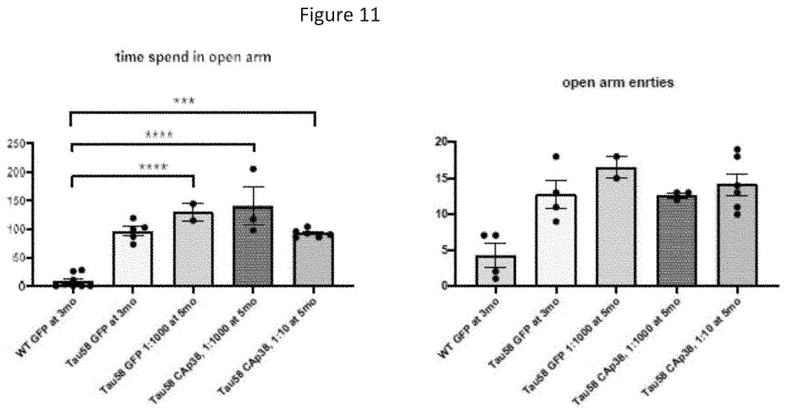

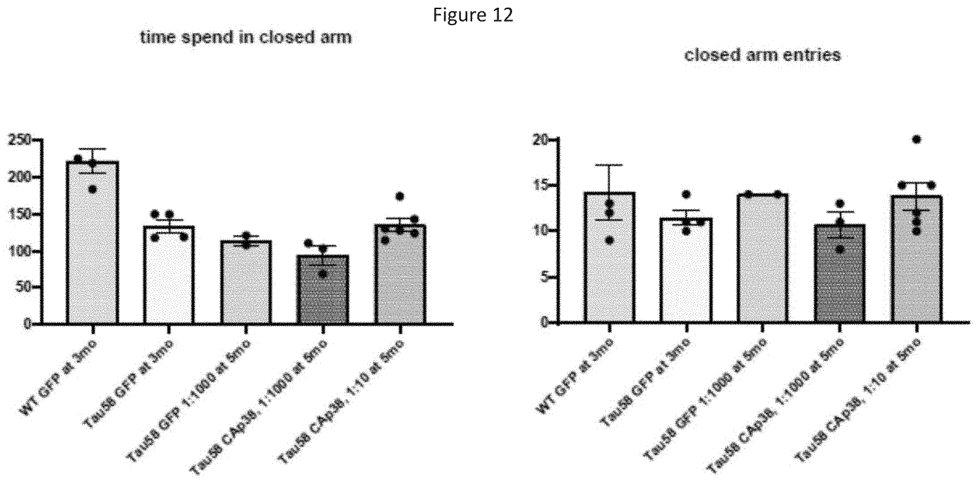

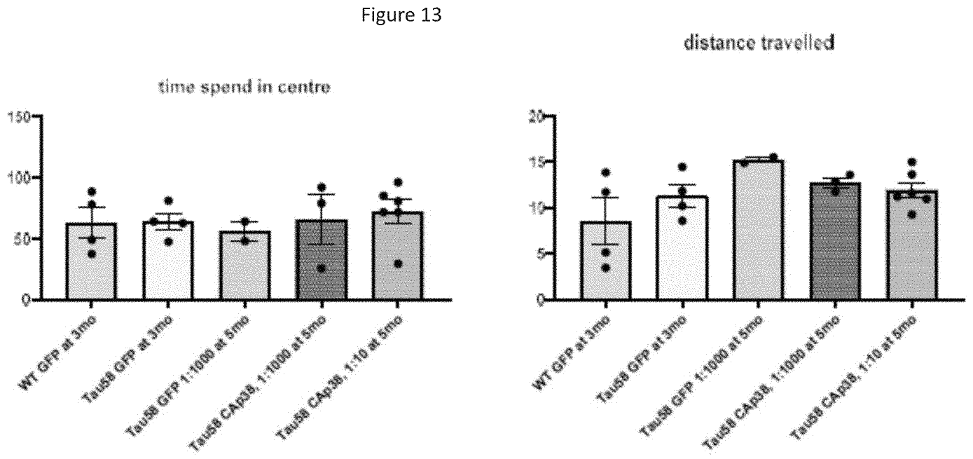

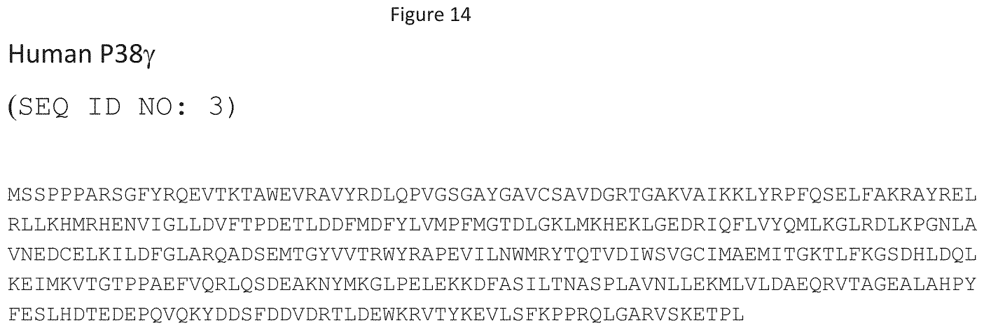

In another example, the inventors treated human P301S mutant tau transgenic Tau58 mice with low and high titres of therapeutic AAV-p38γ^{CA} or control AAV-GFP at 3 months of age. Tau58 mice present with a disinhibition phenotype (=increased open arm time) during elevated plus maze (EPM) testing at 3 months of age that further worsens with time. Tau58 mice treated with high titres of AAV-p38γ^{CA} showed reduced open arm time as compared with AAV-GFP-treated Tau58 mice at 5 months of age (FIG. 11). Furthermore, AAV-p38γ^{CA}-treated Tau58 mice showed a titre-dependent improvement of hyperactivity as compared to AAV-GFP-treated Tau58 controls (FIG. 12-13). Taken together, this data demonstrates that the expression of p38γ^{CA} efficiently reverts the deficits associated with tau pathology in a mouse models of tauopathy.

Effect of p38γ on Tau Phosphorylation and Aggregation

To determine the effect of phosphorylation of tau at T205 on tau phosphorylation and aggregation, TAU58/2 mice were treated with AAV-p38γ^{CA} or control (AAV-GFP).

Sequential extraction of brains from AAV-p38γ^{CA} and control (AAV-GFP) treated TAU58/2 mice demonstrated absence of overt tau phosphorylation, and absence of insolubility, upon treatment with p38γ.

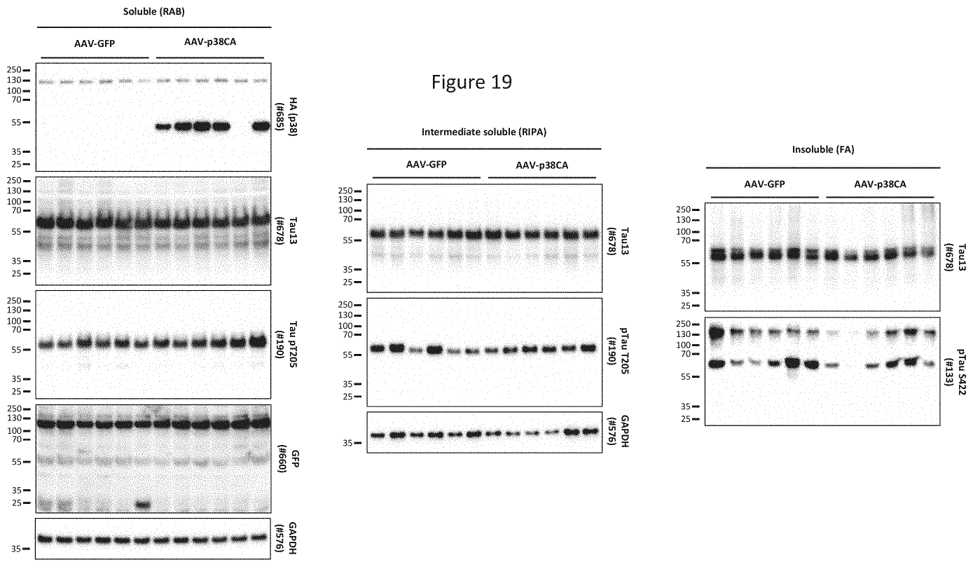

TAU58/2 mice that express P301S mutant tau in brain neurons and form progressive tau pathology reminiscent of human Alzheimer's disease and frontotemporal dementia were injected i.v. with AAV encoding p38γ^{CA} for neuronal expression (operably linked to the human synapsin promoter) or green fluorescence protein (GFP) as a control at 3 months of age. At 11 months of age, all brains were analysed by sequential extraction with buffers of increasing stringency (RAB (0.1 M Mes/1 mM EGTA/0.5 mM MgSO₄, 750 mM NaCl, 20 mM NaF, 1 mM Na₂VO₄, 0.1% roche Protease inhibitor, pH 7.4), RIPA (50 mM Tris/150 mM NaCl/1% Nonidet P-40/5 mM EDTA/0.5% sodium deoxychlolate/0.1% SDS, pH 8.0) and 70% formic acid) (RAB>RIPA>FA) to obtain soluble, intermediate soluble and insoluble proteins respectively. Extracts were analysed by western blotting with HA, GTP, Tau13, GADPH and pTauS422 antibodies. HA antibodies were used to confirm p38γ^{CA} expression and GFP antibodies to identify controls. The results are shown in FIG. 19. Total human transgenic tau was detected with Tau13 antibody, while phosphorylated tau was detected with site-specific antibodies (pTau T205 and pTau S422). We did not find changes (i.e. increases) in tau levels, its phosphorylation or insolubility of tau upon p38γ^{CA} expression, confirming that AAV-p38gCA treatment does not accelerate progression of tau pathology. In fact, phosphorylation of insoluble tau at the late state disease epitope pTau S422 was found to be reduced in insoluble fractions from AAV-p38γ^{CA} treated TAU58/2 mice.

Phosphorylation of tau at serine-422 (pS422) is a disease-related modification, and tau has been found. to undergo increased. phosphorylation at serine 422 in Alzheimer's disease and other tauopathies. Appearance of the pS422 epitope has a strong correlation with neurofibrillary tangle formation and cognitive decline. Accordingly, given the close association between pS422 and cognitive decline, the ability of P38γ to reduce pTau S422 in insoluble fractions of tau from TAU58/2 mouse brains, indicates that p38γ is capable of reversing or reducing cognitive decline and tauopathies associated with, or mediated by, phosphorylation of serine 422 of tau in neurons of the brain.

Taken together, this data shows that p38γ^{CA} expression prevents or reduces tau pathology progression.

### REFERENCES

Delerue, F., and Ittner, L. M. (2017). Generation of Genetically Modified Mice through the Microinjection of Oocytes. J Vis Exp.

Ittner, A., Bertz, J., Suh, L. S., Stevens, C. H., Gotz, J., and Ittner, L. M. (2015). Tau-targeting passive immunization modulates aspects of pathology in tau transgenic mice. J Neurochem 132, 135-145.

Ittner, A., Block, H., Reichel, C. A., Varjosalo, M., Gehart, H., Sumara, G., Gstaiger, M., Krombach, F., Zarbock, A., and Ricci, R. (2012). Regulation of PTEN activity by p38delta-PKD1 signaling in neutrophils confers inflammatory responses in the lung. J Exp Med 209, 2229-2246.

Ittner, A., Chua, S. W., Bertz, J., Volkerling, A., van der Hoven, J., Gladbach, A., Przybyla, M., Bi, M., van Hummel, A., Stevens, C. H., et al. (2016). Site-specific phosphorylation of tau inhibits amyloid-beta toxicity in Alzheimer's mice. Science 354, 904-908.

Ittner, L. M., Ke, Y. D., Delerue, F., Bi, M., Gladbach, A., van Eersel, J., Wolfing, H., Chieng, B. C., Christie, M. J., Napier, I .A., et al. (2010). Dendritic Function of Tau Mediates Amyloid-beta Toxicity in Alzheimer's Disease Mouse Models. Cell 142, 387-397.

Ke, Y. D., van Hummel, A., Stevens, C. H., Gladbach, A., Ippati, S., Bi, M., Lee, W. S., Kruger, S., van der Hoven, J., Volkerling, A., et al. (2015). Short-term suppression of A315T mutant human TDP-43 expression improves functional deficits in a novel inducible transgenic mouse model of FTLD-TDP and ALS. Acta Neuropathol 130, 661-678.

Probst, A., Gotz, J., Wiederhold, K. H., Tolnay, M., Mistl, C., Jaton, A. L., Hong, M., Ishihara, T., Lee, V. M., Trojanowski, J. Q., et al. (2000). Axonopathy and amyotrophy in mice transgenic for human four-repeat tau protein. Acta Neuropathol (Berl) 99, 469-481.

Sturchler-Pierrat, C., Abramowski, D., Duke, M., Wiederhold, K. H., Mistl, C., Rothacher, S., Ledermann, B., Burki, K., Frey, P., Paganetti, P. A., et al. (1997). Two amyloid precursor protein transgenic mouse models with Alzheimer disease-like pathology. Proc Natl Acad Sci U S A 94, 13287-13292.

Tucker, K. L., Meyer, M., and Barde, Y. A. (2001). Neurotrophins are required for nerve growth during development. Nat Neurosci 4, 29-37.

van Eersel, J., Stevens, C. H., Przybyla, M., Gladbach, A., Stefanoska, K., Chan, C. K., Ong, W. Y., Hodges, J. R., Sutherland, G. T., Kril, J. J., et al. (2015). Early-onset axonal pathology in a novel P301S-Tau transgenic mouse model of frontotemporal lobar degeneration. Neuropathology and applied neurobiology 41, 906-925.

Yang, H., Wang, H., and Jaenisch, R. (2014). Generating genetically modified mice using CRISPR/Cas-mediated genome engineering. Nat Protoc 9, 1956-1968.

1. A method of treating or preventing a tauopathy in a subject, comprising administering an agent which:
(a) promotes phosphorylation of tau at threonine in the sequence SSPGSPGTPGSRSR of the tau; and/or
(b) introduces a phosphomimetic of a phosphorylated tau, wherein the phosphorylated tau is tau that has been phosphorylated at threonine in the sequence SSPGSPGTPGSRSR of the tau.

2. The method of claim 1, wherein the subject is suffering from cognitive impairment associated with the tauopathy.

3. The method of claim 1, which comprises reducing or preventing tau aggregation in neurons of the subject.

4. The method of claim 3, wherein the tau aggregation is neurofibrillary tangles.

5. The method of claim 1, which comprises reducing phosphorylation of serine at position 422 of human tau in neurons of the subject.

6. The method of claim 1, wherein the threonine in the sequence SSPGSPGTPGSRSR of tau is threonine at position 205 of human tau.

7. The method of claim 1, wherein the tauopathy is associated with phosphorylation of serine at position 422 of human tau.

8. The method of claim 1, wherein phosphorylation of tau is promoted by administering an agent that elevates p38γ activity, or the activity of a variant of p38γ, in neurons of the subject.

9. The method of claim 8, wherein the agent comprises p38γ, or a variant thereof.

10. The method of claim 8, wherein the agent comprises a nucleic acid that is capable of expressing p38γ, or a variant thereof, in neurons of the subject.

11. The method of claim 8, wherein p38γ comprises the amino acid sequence of SEQ ID NO: 2.

12. The method of claim 8, wherein the variant of p38γ comprises an amino acid sequence that is at least 60%, 65%, 70%, 75%, 80%, 85%, 90%, 95% or 99% identical to the amino acid sequence of SEQ ID NO: 2.

13. The method of claim 8, wherein the variant of p38γ comprises an amino acid sequence selected from the group consisting of: ETPL, KETPL, SKETPL, VSKETPL, RVSKETPL, ARVSKETPL, GARVSKETPL, LGARVSKETPL, QLGARVSKETPL, RQLGARVSKETPL, PRQLGARVSKETPL, PPRQLGARVSKETPL, KPPRQLGARVSKETPL, FKPPRQLGARVSKETPL, SFKPPRQLGARVSKETPL, LSFKPPRQLGARVSKETPL, VLSFKPPRQLGARVSKETPL, EVLSFKPPRQLGARVSKETPL, KEVLSFKPPRQLGARVSKETPL, YKEVLSFKPPRQLGARVSKETPL, TYKE VLSFKPPRQLGARVSKETPL, VTYKEVLSFKPPRQLGARVSKETPL, RVTYKEVLSFKPPRQLGARVSKETPL, KRVTYKEVLSFKPPRQLGARVSKETPL, ETAL, KETAL, PKETAL, VPKETAL, RVPKETAL, ARVPKETAL, GARVPKETAL, LGARVPKETAL, QLGARVPKETAL, RQLGARVPKETAL, PRQLGARVPKETAL, PPRQLGARVPKETAL, KPPRQLGARVPKETAL, FKPPRQLGARVPKETAL, SFKPPRQLGARVPKETAL, LSFKPPRQLGARVPKETAL, VLSFKPPRQLGARVPKETAL, EVLSFKPPRQLGARVPKETAL, KEVLSFKPPRQLGARVPKETAL, YKEVLSFKPPRQLGARVPKETAL, TYKEVLSFKPPRQLGARVPKETAL, VTYKEVLSFKPPRQLGARVPKETAL, RVTYKEVLSFKPPRQLGARVPKETAL, and KRVTYKEVLSFKPPRQLGARVPKETAL.

14. The method of claim 8, wherein the variant of p38γ is a constitutively active variant of p38γ.

15. The method of claim 14, wherein the constitutively active variant of p38γ (p38γ^{CA}) comprises SEQ ID NO: 3.

16. The method of claim 1, wherein the phosphomimetic of phosphorylated tau is introduced into neurons of the subject by introducing into neurons of the subject a tau gene editing system.

17. The method of claim 16, wherein the tau gene editing system introduces into the tau gene a substitution of threonine for glutamic acid at position 205 of human tau.

18. The method of claim 16, wherein the gene editing system is a CRISPR/Cas complex, or a part thereof.

19. The method of claim 18, wherein the CRISPR/Cas complex is a CRISPR/Cas9 complex.

20. The method of claim 1, wherein the tauopathy is advanced Alzheimer's disease, frontotemporal lobar dementia, corticobasal degeneration, progressive supranuclear palsy, primary age-related tauopathy, chronic traumatic encephalopathy, frontotemporal dementia with parkinsonism linked to chromosome 17, Pick's disease, globular glial tauopathy, or Parkinson's disease.

21. The method of claim 2, wherein the improvement in cognitive ability is an improvement in memory.

22. An agent comprising a tau (Mapt) gene editing system, or part therefor, or a nucleic acid encoding a tau gene editing system, or part thereof, the gene editing system or part thereof comprising a nucleic acid which introduces a mutation into the wild-type tau gene to cause expression of a phosphomimetic of phosphorylated tau, wherein the phosphorylated tau has been phosphorylated at threonine in the sequence SSPGSPGTPGSRSR of the tau.

23. The agent of claim 22, wherein the tau gene editing system is a CRISPR/Cas9 system comprising a gRNA which targets nucleotide sequence at or near threonine in the sequence SSPGSPGTPGSRSR of the tau.

24. A composition comprising the agent of claim 22.


SEQUENCE LISTING (109 sequences)

SEQ ID NO: 1 - protein, 441 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 2 - RNA, 2280 nt, Homo sapiens
caggcucugcggggugggcagcucccgggccugccaugagcucuccgccgcccgcccgcaguggcuuuuaccgccaggaggugaccaagacggccugggaggugcgcgccguguaccgggaccugcagcccgugggcucgggcgccuacggcgcggugugcucggccguggacggccgcaccggcgcuaagguggccaucaagaagcuguaucggcccuuccaguccgagcuguucgccaagcgcgccuaccgcgagcugcgccugcucaagcacaugcgccacgagaacgugaucgggcugcuggacguauucacuccugaugagacccuggaugacuucauggacuuuuaccuggugaugccguucaugggcaccgaccugggcaagcucaugaaacaugagaagcuaggcgaggaccggauccaguuccucguguaccagaugcugaaggggcugagggaccugaagcccggcaaccuggcugugaacgaagacugugagcugaagauccuggacuucggccuggccaggcaggcagacagugagaugacuggguacguggugacccggugguaccgggcucccgaggucaucuugaauuggaugcgcuacacgcagacgguggacaucugguccgugggcugcaucauggcggagaugaucacaggcaagacgcuguucaagggcagcgaccaccuggaccagcugaaggagaucaugaaggugacggggacgccuccggcugaguuugugcagcggcugcagagcgaugaggccaagaacuacaugaagggccuccccgaauuggagaagaaggauuuugccucuauccugaccaaugcaagcccucuggcugugaaccuccuggagaagaugcuggugcuggacgcggagcagcgggugacggcaggcgaggcgcuggcccaucccuacuucgagucccugcacgacacggaagaugagccccagguccagaaguaugaugacuccuuugacgacguugaccgcacacuggaugaauggaagcguguuacuuacaaagaggugcucagcuucaagccuccccggcagcugggggccagggucuccaaggagacgccucugugaagaucucugggcuccgggguggcagugaggacaggcucugcggggugggcagcucccgggccugccaugagcucuccgccgcccgcccgcaguggcuuuuaccgccaggaggugaccaagacggccugggaggugcgcgccguguaccgggaccugcagcccgugggcucgggcgccuacggcgcggugugcucggccguggacggccgcaccggcgcuaagguggccaucaagaagcuguaucggcccuuccaguccgagcuguucgccaagcgcgccuaccgcgagcugcgccugcucaagcacaugcgccacgagaacgugaucgggcugcuggacguauucacuccugaugagacccuggaugacuucauggacuuuuaccuggugaugccguucaugggcaccgaccugggcaagcucaugaaacaugagaagcuaggcgaggaccggauccaguuccucguguaccagaugcugaaggggcugagggaccugaagcccggcaaccuggcugugaacgaagacugugagcugaagauccuggacuucggccuggccaggcaggcagacagugagaugacuggguacguggugacccggugguaccgggcucccgaggucaucuugaauuggaugcgcuacacgcagacgguggacaucugguccgugggcugcaucauggcggagaugaucacaggcaagacgcuguucaagggcagcgaccaccuggaccagcugaaggagaucaugaaggugacggggacgccuccggcugaguuugugcagcggcugcagagcgaugaggccaagaacuacaugaagggccuccccgaauuggagaagaaggauuuugccucuauccugaccaaugcaagcccucuggcugugaaccuccuggagaagaugcuggugcuggacgcggagcagcgggugacggcaggcgaggcgcuggcccaucccuacuucgagucccugcacgacacggaagaugagccccagguccagaaguaugaugacuccuuugacgacguugaccgcacacuggaugaauggaagcguguuacuuacaaagaggugcucagcuucaagccuccccggcagcugggggccagggucuccaaggagacgccucugugaagaucucugggcuccgggguggcagugagga

SEQ ID NO: 3 - protein, 357 aa, Homo sapiens
MSSPPPARSGFYRQEVTKTAWEVRAVYRDLQPVGSGAYGAVCSAVDGRTGAKVAIKKLYRPFQSELFAKRAYRELRLLKHMRHENVIGLLDVFTPDETLDDFMDFYLVMPFMGTDLGKLMKHEKLGEDRIQFLVYQMLKGLRDLKPGNLAVNEDCELKILDFGLARQADSEMTGYVVTRWYRAPEVILNWMRYTQTVDIWSVGCIMAEMITGKTLFKGSDHLDQLKEIMKVTGTPPAEFVQRLQSDEAKNYMKGLPELEKKDFASILTNASPLAVNLLEKMLVLDAEQRVTAGEALAHPYFESLHDTEDEPQVQKYDDSFDDVDRTLDEWKRVTYKEVLSFKPPRQLGARVSKETPL

SEQ ID NO: 4 - protein, 357 aa, Artificial Sequence
  D179A mutant of human P38gamma
MSSPPPARSGFYRQEVTKTAWEVRAVYRDLQPVGSGAYGAVCSAVDGRTGAKVAIKKLYRPFQSELFAKRAYRELRLLKHMRHENVIGLLDVFTPDETLDDFMDFYLVMPFMGTDLGKLMKHEKLGEDRIQFLVYQMLKGLRDLKPGNLAVNEDCELKILDFGLARQAASEMTGYVVTRWYRAPEVILNWMRYTQTVDIWSVGCIMAEMITGKTLFKGSDHLDQLKEIMKVTGTPPAEFVQRLQSDEAKNYMKGLPELEKKDFASILTNASPLAVNLLEKMLVLDAEQRVTAGEALAHPYFESLHDTEDEPQVQKYDDSFDDVDRTLDEWKRVTYKEVLSFKPPRQLGARVSKETPL

SEQ ID NO: 5 - protein, 441 aa, Artificial Sequence
  mutant human tau T205E
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGEPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 6 - DNA, 120 bp, Artificial Sequence
  Donor sequence for mutating human tau
tctggtgaacctccaaaatcaggggatcgcagcggctacagcagccccggctccccaggcgagcccggcagccgctcccgcaccccgtcccttccaaccccacccacccgggagcccaag

SEQ ID NO: 7 - protein, 14 aa, Homo sapiens
SSPGSPGTPGSRSR

SEQ ID NO: 8 - protein, 14 aa, Artificial Sequence
  Portion of mutated tau
  MISC_FEATURE (8)..(8): X is D or E
SSPGSPGXPGSRSR

SEQ ID NO: 9 - protein, 4 aa, Artificial Sequence
  Portion of p38g
ETPL

SEQ ID NO: 10 - protein, 5 aa, Artificial Sequence
  portion of p38gamma
KETPL

SEQ ID NO: 11 - protein, 6 aa, Artificial Sequence
  portion of p38gamma
SKETPL

SEQ ID NO: 12 - protein, 7 aa, Artificial Sequence
  portion of p38gamma
VSKETPL

SEQ ID NO: 13 - protein, 8 aa, Artificial Sequence
  portion of p38gamma
RVSKETPL

SEQ ID NO: 14 - protein, 9 aa, Artificial Sequence
  ARVSKETPL
ARVSKETPL

SEQ ID NO: 15 - protein, 10 aa, Artificial Sequence
  portion of p38gamma
GARVSKETPL

SEQ ID NO: 16 - protein, 11 aa, Artificial Sequence
  portion of p36gamma
LGARVSKETPL

SEQ ID NO: 17 - protein, 12 aa, Artificial Sequence
  portion of p38gamma
QLGARVSKETPL

SEQ ID NO: 18 - protein, 13 aa, Artificial Sequence
  portion of p38gamma
RQLGARVSKETPL

SEQ ID NO: 19 - protein, 14 aa, Artificial Sequence
  portion p38gamma
PRQLGARVSKETPL

SEQ ID NO: 20 - protein, 15 aa, Artificial Sequence
  portion of p38gamma
PPRQLGARVSKETPL

SEQ ID NO: 21 - protein, 16 aa, Artificial Sequence
  portion p38gamma
KPPRQLGARVSKETPL

SEQ ID NO: 22 - protein, 17 aa, Artificial Sequence
  portion of p38gamma
FKPPRQLGARVSKETPL

SEQ ID NO: 23 - protein, 18 aa, Artificial Sequence
  portion of p38gamma
SFKPPRQLGARVSKETPL

SEQ ID NO: 24 - protein, 19 aa, Artificial Sequence
  portion of p38gamma
LSFKPPRQLGARVSKETPL

SEQ ID NO: 25 - protein, 20 aa, Artificial Sequence
  portion of p38gamma
VLSFKPPRQLGARVSKETPL

SEQ ID NO: 26 - protein, 21 aa, Artificial Sequence
  portion of p38gamma
EVLSFKPPRQLGARVSKETPL

SEQ ID NO: 27 - protein, 22 aa, Artificial Sequence
  portion of p38gamma
KEVLSFKPPRQLGARVSKETPL

SEQ ID NO: 28 - protein, 23 aa, Artificial Sequence
  portion of p38gamma
YKEVLSFKPPRQLGARVSKETPL

SEQ ID NO: 29 - protein, 24 aa, Artificial Sequence
  portion of p38gamma
TYKEVLSFKPPRQLGARVSKETPL

SEQ ID NO: 30 - protein, 25 aa, Artificial Sequence
  portion of p38gamma
VTYKEVLSFKPPRQLGARVSKETPL

SEQ ID NO: 31 - protein, 26 aa, Artificial Sequence
  portion of p38gamma
RVTYKEVLSFKPPRQLGARVSKETPL

SEQ ID NO: 32 - protein, 27 aa, Artificial Sequence
  portion of p38gamma
KRVTYKEVLSFKPPRQLGARVSKETPL

SEQ ID NO: 33 - protein, 4 aa, Artificial Sequence
  portion of p38gamma
ETAL

SEQ ID NO: 34 - protein, 5 aa, Artificial Sequence
  portion of p38gamma
KETAL

SEQ ID NO: 35 - protein, 6 aa, Artificial Sequence
  portion of p38gamma
PKETAL

SEQ ID NO: 36 - protein, 7 aa, Artificial Sequence
  portion of p38gamma
VPKETAL

SEQ ID NO: 37 - protein, 8 aa, Artificial Sequence
  portion of p38gamma
RVPKETAL

SEQ ID NO: 38 - protein, 9 aa, Artificial Sequence
  portion of p38gamma
ARVPKETAL

SEQ ID NO: 39 - protein, 10 aa, Artificial Sequence
  portion of p38gamma
GARVPKETAL

SEQ ID NO: 40 - protein, 11 aa, Artificial Sequence
  portion of p38gamma
LGARVPKETAL

SEQ ID NO: 41 - protein, 12 aa, Artificial Sequence
  portion of p38gamma
QLGARVPKETAL

SEQ ID NO: 42 - protein, 13 aa, Artificial Sequence
  portion of p38gamma
RQLGARVPKETAL

SEQ ID NO: 43 - protein, 14 aa, Artificial Sequence
  portion of p38gamma
PRQLGARVPKETAL

SEQ ID NO: 44 - protein, 15 aa, Artificial Sequence
  portion of p38gamma
PPRQLGARVPKETAL

SEQ ID NO: 45 - protein, 16 aa, Artificial Sequence
  portion of p38gamma
KPPRQLGARVPKETAL

SEQ ID NO: 46 - protein, 17 aa, Artificial Sequence
  portion of p38gamma
FKPPRQLGARVPKETAL

SEQ ID NO: 47 - protein, 18 aa, Artificial Sequence
  portion of p38gamma
SFKPPRQLGARVPKETAL

SEQ ID NO: 48 - protein, 19 aa, Artificial Sequence
  portion of p38gamma
LSFKPPRQLGARVPKETAL

SEQ ID NO: 49 - protein, 20 aa, Artificial Sequence
  portion of p38gamma
VLSFKPPRQLGARVPKETAL

SEQ ID NO: 50 - protein, 21 aa, Artificial Sequence
  portion of p38gamma
EVLSFKPPRQLGARVPKETAL

SEQ ID NO: 51 - protein, 22 aa, Artificial Sequence
  portion of p38gamma
KEVLSFKPPRQLGARVPKETAL

SEQ ID NO: 52 - protein, 23 aa, Artificial Sequence
  portion of p38gamma
YKEVLSFKPPRQLGARVPKETAL

SEQ ID NO: 53 - protein, 24 aa, Artificial Sequence
  portion of p38 gamma
TYKEVLSFKPPRQLGARVPKETAL

SEQ ID NO: 54 - protein, 25 aa, Artificial Sequence
  portion of p38gamma
VTYKEVLSFKPPRQLGARVPKETAL

SEQ ID NO: 55 - protein, 26 aa, Artificial Sequence
  portion of p38gamma
RVTYKEVLSFKPPRQLGARVPKETAL

SEQ ID NO: 56 - protein, 27 aa, Artificial Sequence
  portion of p38gamma
KRVTYKEVLSFKPPRQLGARVPKETAL

SEQ ID NO: 57 - protein, 11 aa, Artificial Sequence
  portion of constitutively active mutant of p38gamma
ARQAASEMTGY

SEQ ID NO: 58 - protein, 13 aa, Artificial Sequence
  portion of constitutively active mutant of p38gamma
LARQAASEMTGYV

SEQ ID NO: 59 - protein, 20 aa, Artificial Sequence
  portion of constitutively active mutant of p38gamma
DFGLARQAASEMTGYVVTRW

SEQ ID NO: 60 - protein, 40 aa, Artificial Sequence
  portion of constitutively active mutant of p38gamma
VNEDCELKILDFGLARQAASEMTGYVVTRWYRAPEVILNW

SEQ ID NO: 61 - protein, 60 aa, Artificial Sequence
  portion of constitutively active mutant of p38gamma
HRDLKPGNLAVNEDCELKILDFGLARQAASEMTGYVVTRWYRAPEVILNWMRYTQTVDIW

SEQ ID NO: 62 - protein, 80 aa, Artificial Sequence
  portion of constitutively active mutant of p38gamma
LRYIHAAGIIHRDLKPGNLAVNEDCELKILDFGLARQAASEMTGYVVTRWYRAPEVILNWMRYTQTVDIWSVGCIMAEMI

SEQ ID NO: 63 - protein, 100 aa, Artificial Sequence
  portion of constitutive mutant of p38gamma
QFLVYQMLKGLRYIHAAGIIHRDLKPGNLAVNEDCELKILDFGLARQAASEMTGYVVTRWYRAPEVILNWMRYTQTVDIWSVGCIMAEMITGKTLFKGSD

SEQ ID NO: 64 - protein, 120 aa, Artificial Sequence
  portion of constitutive mutant of p38gamma
KHEKLGEDRIQFLVYQMLKGLRYIHAAGIIHRDLKPGNLAVNEDCELKILDFGLARQAASEMTGYVVTRWYRAPEVILNWMRYTQTVDIWSVGCIMAEMITGKTLFKGSDHLDQLKEIMK

SEQ ID NO: 65 - protein, 140 aa, Artificial Sequence
  portion of constitutive mutant of p38gamma
FMGTDLGKLMKHEKLGEDRIQFLVYQMLKGLRYIHAAGIIHRDLKPGNLAVNEDCELKILDFGLARQAASEMTGYVVTRWYRAPEVILNWMRYTQTVDIWSVGCIMAEMITGKTLFKGSDHLDQLKEIMKVTGTPPAEFV

SEQ ID NO: 66 - protein, 160 aa, Artificial Sequence
  portion of constitutive mutant of p38gamma
DFTDFYLVMPFMGTDLGKLMKHEKLGEDRIQFLVYQMLKGLRYIHAAGIIHRDLKPGNLAVNEDCELKILDFGLARQAASEMTGYVVTRWYRAPEVILNWMRYTQTVDIWSVGCIMAEMITGKTLFKGSDHLDQLKEIMKVTGTPPAEFVQRLQSDEAKN

SEQ ID NO: 67 - protein, 180 aa, Artificial Sequence
  portion of constitutive mutant of p38gamma
DVFTPDETLDDFTDFYLVMPFMGTDLGKLMKHEKLGEDRIQFLVYQMLKGLRYIHAAGIIHRDLKPGNLAVNEDCELKILDFGLARQAASEMTGYVVTRWYRAPEVILNWMRYTQTVDIWSVGCIMAEMITGKTLFKGSDHLDQLKEIMKVTGTPPAEFVQRLQSDEAKNYMKGLPELEK

SEQ ID NO: 68 - protein, 200 aa, Artificial Sequence
  portion of constitutive mutant of p38gamma
MRHENVIGLLDVFTPDETLDDFTDFYLVMPFMGTDLGKLMKHEKLGEDRIQFLVYQMLKGLRYIHAAGIIHRDLKPGNLAVNEDCELKILDFGLARQAASEMTGYVVTRWYRAPEVILNWMRYTQTVDIWSVGCIMAEMITGKTLFKGSDHLDQLKEIMKVTGTPPAEFVQRLQSDEAKNYMKGLPELEKKDFASILTNA

SEQ ID NO: 69 - DNA, 20 bp, Artificial Sequence
  Guide DNA
cgagcgactgccaggcgttc

SEQ ID NO: 70 - DNA, 20 bp, Artificial Sequence
  Guide DNA
ggagcggctgccgggagtgc

SEQ ID NO: 71 - DNA, 20 bp, Artificial Sequence
  Guide DNA
cccggctctcccggaacgcc

SEQ ID NO: 72 - DNA, 20 bp, Artificial Sequence
  Guide DNA
cccggctccccaggcactcc

SEQ ID NO: 73 - DNA, 120 bp, Artificial Sequence
  Donor sequence for CRISPR
ccaggtgaaccaccaaaatccggagaacgaagcggctacagcagccccggctctcccggagagcctggcagtcgctcgcgcaccccatccctaccaacaccgcccacccgggagcccaag

SEQ ID NO: 74 - protein, 13 aa, Artificial Sequence
  portion of mutant tau
  MISC_FEATURE (7)..(7): X is Dor E
SPGSPGXPGSRSR

SEQ ID NO: 75 - protein, 15 aa, Artificial Sequence
  portion of mutant tau
  MISC_FEATURE (8)..(8): X is D or E
SSPGSPGXPGSRSRT

SEQ ID NO: 76 - protein, 15 aa, Artificial Sequence
  portion of mutant tau
  MISC_FEATURE (8)..(8): X is D or E
SSPGSPGXPGSRSRT

SEQ ID NO: 77 - protein, 17 aa, Artificial Sequence
  portion of mutant tau
  MISC_FEATURE (9)..(9): X is D or E
YSSPGSPGXPGSRSRTP

SEQ ID NO: 78 - protein, 19 aa, Artificial Sequence
  portion of mutant tau
  MISC_FEATURE (10)..(10): X is D or E
GYSSPGSPGXPGSRSRTPS

SEQ ID NO: 79 - protein, 21 aa, Artificial Sequence
  portion of mutant tau
  MISC_FEATURE (11)..(11): X is D or E
SGYSSPGSPGXPGSRSRTPSL

SEQ ID NO: 80 - protein, 23 aa, Artificial Sequence
  portion of mutant tau
  MISC_FEATURE (12)..(12): X is D or E
RSGYSSPGSPGXPGSRSRTPSLP

SEQ ID NO: 81 - protein, 25 aa, Artificial Sequence
  portion of mutant tau
  MISC_FEATURE (13)..(13): X is D or E
DRSGYSSPGSPGXPGSRSRTPSLPT

SEQ ID NO: 82 - protein, 27 aa, Artificial Sequence
  portion of mutant tau
  MISC_FEATURE (14)..(14): X is D or E
GDRSGYSSPGSPGXPGSRSRTPSLPTP

SEQ ID NO: 83 - protein, 29 aa, Artificial Sequence
  portion of mutant tau
  MISC_FEATURE (15)..(15): X is D or E
SGDRSGYSSPGSPGXPGSRSRTPSLPTPP

SEQ ID NO: 84 - protein, 31 aa, Artificial Sequence
  portion of mutant tau
  MISC_FEATURE (16)..(16): X is D or E
KSGDRSGYSSPGSPGXPGSRSRTPSLPTPPT

SEQ ID NO: 85 - protein, 33 aa, Artificial Sequence
  portion of mutant tau
  MISC_FEATURE (17)..(17): X is D or E
PKSGDRSGYSSPGSPGXPGSRSRTPSLPTPPTR

SEQ ID NO: 86 - protein, 35 aa, Artificial Sequence
  portion of mutant tau
  MISC_FEATURE (18)..(18): X is D or E
PPKSGDRSGYSSPGSPGXPGSRSRTPSLPTPPTRE

SEQ ID NO: 87 - protein, 37 aa, Artificial Sequence
  portion of mutant tau
  MISC_FEATURE (19)..(19): X is D or E
EPPKSGDRSGYSSPGSPGXPGSRSRTPSLPTPPTREP

SEQ ID NO: 88 - protein, 39 aa, Artificial Sequence
  portion of mutant tau
  MISC_FEATURE (20)..(20): X is D or E
GEPPKSGDRSGYSSPGSPGXPGSRSRTPSLPTPPTREPK

SEQ ID NO: 89 - protein, 40 aa, Artificial Sequence
  portion of mutant tau
  MISC_FEATURE (21)..(21): X is D or E
SGEPPKSGDRSGYSSPGSPGXPGSRSRTPSLPTPPTREPK

SEQ ID NO: 90 - DNA, 20 bp, Artificial Sequence
  Primer sequence
gttctgctgcatcttggaca

SEQ ID NO: 91 - DNA, 20 bp, Artificial Sequence
  Primer sequence
gaattccgacatgactcagg

SEQ ID NO: 92 - DNA, 27 bp, Artificial Sequence
  Primer sequence
gggtgtctccaatgcctgcttcttcag

SEQ ID NO: 93 - DNA, 26 bp, Artificial Sequence
  Primer sequence
aagtcacccagcagggaggtgctcag

SEQ ID NO: 94 - DNA, 21 bp, Artificial Sequence
  Primer sequence
tgggctgcgaaggtagaggtg

SEQ ID NO: 95 - DNA, 21 bp, Artificial Sequence
  Primer sequence
gtgtcacgtgctcagggcctg

SEQ ID NO: 96 - DNA, 20 bp, Artificial Sequence
  Primer sequence
ctcagcatcccacctgtaac

SEQ ID NO: 97 - DNA, 20 bp, Artificial Sequence
  Primer sequence
ccagttgtgtatgtccaccc

SEQ ID NO: 98 - DNA, 19 bp, Artificial Sequence
  Primer sequence
aagttcatctgcaccaccg

SEQ ID NO: 99 - DNA, 20 bp, Artificial Sequence
  Primer sequence
tgctcaggtagtggttgtcg

SEQ ID NO: 100 - DNA, 21 bp, Artificial Sequence
  Primer sequence
aagtcacccagcagggaggtg

SEQ ID NO: 101 - DNA, 22 bp, Artificial Sequence
  Primer sequence
tcgtatgggtacatggccaaag

SEQ ID NO: 102 - DNA, 20 bp, Artificial Sequence
  Primer sequence
cagccccggctctcccgtag

SEQ ID NO: 103 - DNA, 20 bp, Artificial Sequence
  Primer sequence
cgcgagcgactgccaggagt

SEQ ID NO: 104 - DNA, 30 bp, Artificial Sequence
  Primer sequence
tgtatcaaagtgacagagcaggagtgatgc

SEQ ID NO: 105 - DNA, 28 bp, Artificial Sequence
  Primer sequence
tggtgcttcaggttctcagtagagccaa

SEQ ID NO: 106 - DNA, 28 bp, Artificial Sequence
  Primer sequence
cctgaaagaagatcccctgcagaccccc

SEQ ID NO: 107 - DNA, 18 bp, Artificial Sequence
  Primer sequence
ccagcgtccgtgtcaccc

SEQ ID NO: 108 - DNA, 47 bp, Artificial Sequence
  Primer sequence
caagcccagcaatgcctacctgagtgacgtgagcaagggcgaggagg

SEQ ID NO: 109 - DNA, 51 bp, Artificial Sequence
  Primer sequence
cgaggttgtgatgtctgggggagcatagctcttgtacagctcgtccatgcc